(19) United States
(12) Patent Application Publication
(10) Pub. No.: US 2025/0166734 A1
(43) Pub. Date: May 22, 2025

(54) MACHINE LEARNING SYSTEMS AND METHODS FOR SOMATIC MUTATION DETECTION

(71) Applicant: Personal Genome Diagnotics Inc., Baltimore, MD (US)

(72) Inventors: Samuel V. Angiuoli, Baltimore, MD (US); Derrick Wood, Baltimore, MD (US); Andrew Georgiadis, Baltimore, MD (US); Mark Sausen, Baltimore, MD (US); James R. White, Baltimore, MD (US); David Riley, Baltimore, MD (US)

(73) Assignee: Personal Genome Diagnostics Inc., Baltimore, MD (US)

(21) Appl. No.: 19/031,137

(22) Filed: Jan. 17, 2025

Related U.S. Application Data
(63) Continuation-in-part of application No. 18/619,485, filed on Mar. 28, 2024.
Application No. 18/619,485 is a continuation-in-part of application No. 16/217,921, filed on Dec. 12, 2018, now Pat. No. 11,972,841.
(60) Provisional application No. 62/726,877, filed on Sep. 4, 2018.
(60) Provisional application No. 62/607,007, filed on Dec. 18, 2017.

(51) Int. Cl. G16B 30/10; G16B 40/20

(52) U.S. Cl. CPC G16B 30/10; G16B 40/20

(57) ABSTRACT

The present disclosure relates to somatic mutation determinations. An example method includes performing whole genome sequencing using a high-throughput sequencing system on nucleic acids from a sample obtained from a subject to generate raw data, transmitting the raw data from the high-throughput sequencing system to a computing system, processing the raw data by the computing system to generate candidate variants and a set of feature values for each candidate variant of the candidate variants, processing the feature values by the computing system using a trained machine learning model to identify somatic mutations from the candidate variants, transmitting results based on the somatic mutations from the computing system to an end device, and displaying a report on the end device based on the results.

### CROSS-REFERENCE TO RELATED APPLICATIONS

The present application is a continuation-in-part of and claims benefit and priority to U.S. application Ser. No. 18/619,485, filed Mar. 28, 2024, which claims benefit and priority to U.S. application Ser. No. 16/217,921, filed Dec. 12, 2018, (now U.S. Pat. No. 11,972,841), which claims benefit and priority to U.S. Application Ser. No. 62/726,877, filed Sep. 4, 2018, and to U.S. Application Ser. No. 62/607,007, filed Dec. 18, 2017, the entire contents of each of which are incorporated herein by reference for all purposes.

### FIELD

The present disclosure relates to a Next Generation Sequencing clinical genetic screening assay that leverages a machine learning model to identify tumor-specific mutations in samples from patients with cancer.

### BACKGROUND

Next-generation sequencing (NGS) technologies have transformed routine diagnostics in clinical laboratories worldwide by enabling the rapid and simultaneous sequencing of millions of DNA fragments. Among the most widely used NGS techniques are Whole Genome Sequencing (WGS) and Whole Exome Sequencing (WES). WGS involves sequencing the entire genome, which comprises approximately 3 billion base pairs, providing a comprehensive analysis of all genetic material. In contrast, WES targets the exons, the protein-coding regions of the genome, which constitute about 1-2% of the total genome.

The WES process typically begins with the extraction of genomic DNA from a biological sample. The DNA is then fragmented, and sequencing adapters are added to create a sequencing library. This library is enriched for exonic regions using techniques such as hybrid capture or targeted amplification, ensuring that the protein-coding regions are selectively isolated. After enrichment, the exonic DNA undergoes amplification, followed by high-throughput sequencing. The resulting data is analyzed using bioinformatics tools to identify genetic variations, including single nucleotide variants (SNVs), insertions, deletions, and other alterations. Since the exome represents only a small fraction of the genome, WES can achieve greater depth-meaning each nucleotide in the exome is sequenced multiple times. WES is widely used to identify genetic alterations associated with various inherited conditions, cancers, and other complex diseases, making it a powerful tool in clinical diagnostics and research.

WGS, on the other hand, does not require hybrid capture preprocessing to enrich for exonic regions. Hybrid capturing can be time-consuming and reagent-intensive, adding to the overall cost and complexity of WES. WGS can offer significant time and resource savings, making it a more practical choice for high-throughput and time-sensitive projects. Furthermore, the efficiency of WGS extends to its ability to process lower quantities of input material. WGS typically requires only 5-10 ng of DNA, whereas WES demands approximately 30 ng, making WGS a viable solution for samples with limited available material. Another efficiency advantage of WGS is its relatively low coverage requirement. Because WGS analyzes a broader range of genetic materials (e.g., the entire genome) and the alterations in both coding and non-coding regions, it can achieve comprehensive results with lower sequencing depth compared to WES, which requires higher coverage to ensure adequate representation of the targeted exonic regions. This lower coverage requirement not only reduces sequencing costs but also accelerates data analysis and processing timelines, further enhancing the time and cost-efficiency of WGS. Because the entire genome is being sequenced, changes in the noncoding, intergenic, and intronic regions of the genome, can also be determined by WGS.

Together, both WGS and WES have been particularly impactful in the field of oncology for detecting tumor-specific (somatic) mutations and aiding oncologists in diagnostic and therapeutic management decisions for their patients. WES and WGS each offer distinct benefits for genomic analysis, depending on the research or clinical objective. WES is a cost-effective and efficient method for identifying genetic variations in protein-coding regions (e.g., exons), which are most likely to harbor disease-causing mutations. By focusing on approximately 1-2% of the genome, WES generates manageable data sizes while enabling the discovery of clinically significant variants for applications such as inherited disease diagnostics, cancer research, and personalized medicine. On the other hand, WGS provides a comprehensive view of the entire genome, including coding and non-coding regions, allowing for a more complete assessment of genetic variations such as structural variants, intronic mutations, and regulatory elements that may influence gene expression or contribute to complex diseases. While WES is ideal for targeted studies of known functional regions, WGS is particularly valuable for uncovering novel variants (e.g., when the source of the cancer is unknown) and gaining insights into broader genomic contexts, making the two approaches complementary tools for advancing precision medicine and genetic research.

Although NGS has become a cornerstone technology for identifying genetic mutations and been widely used in both research and clinical settings for applications like cancer genomics, inherited disease diagnostics, and personalized medicine, it has notable limitations that can complicate mutation detection. Somatic mutations, for instance, often occur at low allelic frequencies, especially in heterogeneous samples such as tumors, where mutant and normal cells coexist. Detecting these low-frequency mutations requires deep sequencing coverage and sophisticated bioinformatics tools to differentiate true mutations from sequencing errors and noise. Additionally, the short-read lengths generated by NGS platforms can pose difficulties in accurately mapping mutations in regions with repetitive sequences, high GC content, or complex structural variations. These challenges are further magnified when working with degraded or low-quality DNA, such as that derived from formalin-fixed paraffin-embedded (FFPE) tissues, where fragmentation and chemical modifications compromise library quality. Moreover, in mixed samples like liquid biopsies, where target DNA (e.g., tumor-derived DNA or circulating tumor DNA (ctDNA)) is present in low abundance amid a background of non-target DNA, accurate mutation detection becomes even more challenging. These limitations necessitate the use of advanced, error-correction techniques to ensure reliable identification of somatic mutations, particularly in clinical and diagnostic contexts.

### BRIEF SUMMARY

Disclosed are methods, systems, and computer readable storage media for Next Generation Sequencing clinical genetic screening assay that leverages machine learning models to identify somatic mutations. The methods, systems, and computer readable storage media may be embodied in a variety of ways.

In various embodiments, a computer is trained to recognize sequence mutations or structural alterations from sequence reads. The computer may compare the sequence reads to a reference and, if indicia of a sequence mutation or structural alteration are present, a machine learning algorithm validates that the sequence mutation or structural alteration has been detected. The machine learning algorithm is trained on a training data set that includes sequence reads and known sequence mutations and/or structural alterations. By using the trained machine learning algorithm, the computer can analyze sequence reads from a target nucleic acid and will reliably report when the nucleic acid has a sequence mutation or structural alteration. The machine learning algorithm allows for the detection of sequence mutations or structural alterations when using samples or sequencing technologies that otherwise make such detection difficult.

Systems and methods of the disclosure are particularly useful for detecting sequence mutations or structural alterations in tumor DNA, even when using NGS technologies. NGS sequence reads from tumor DNA are compared to a reference. Where the comparison shows a potential substitution, indel, or rearrangement in the tumor DNA, a machine learning classifier classifies that potential rearrangement as true or a product of experimental noise. The machine learning classifier, such as a random forest or neural network, is trained on training data that includes known sequence mutations or chromosomal alterations. The trained classifier can accurately determine when NGS sequence reads reveal a sequence mutation or structural alteration in a sample. Methods of the disclosure are useful to describe a variety of a complex mutations of distinct types, and may be applied to report genetic information about a tumor when sequencing ctDNA. Sequence reads from a sample are analyzed against a reference and the classification model provides for the detection and accurate reporting of sequence mutations or structural alterations and may be used to report the type, location, or boundaries of such changes.

Using methods of the disclosure, tumor genetics may be analyzed and reported from circulating tumor DNA (ctDNA) obtained from a blood or plasma sample. Methods are useful to describe tumor-specific rearrangements and alterations even using NGS sequencing on ctDNA in a plasma sample. Thus, methods described herein provide a minimally-invasive tool for detecting tumors and monitoring progression or remission.

The classifier can operate within a variant calling pipeline and allow that pipeline to identify tumor-specific mutations, even where a sample may contain a mixture of tumor and normal DNA. The variant calling pipeline can further identify other mutation types such as small indels and substitution mutations. Systems and methods of the disclosure are useful for analyzing ctDNA in plasma samples, and can be used to monitor tumor progression, remission, or treatment.

In certain aspects, the disclosure provides a method for analyzing nucleic acid. The method includes sequencing nucleic acid from a sample from a subject to produce sequence reads and describing a mutation or structural alteration in the nucleic acid using a classification model trained to recognize the structural alteration. Preferably, the nucleic acid is DNA from a tumor, and the method further comprises providing a report that describes the DNA from the tumor as including the mutation or structural alteration. Describing the mutation or structural alteration may include comparing the sequence reads to a reference to detect an indicium of the mutation or structural alteration and validating the mutation or structural alteration as present in the nucleic acid using the classification model. The classification model may be trained on a training data set of sequences that include known mutations and/or structural alterations.

Methods may include training the classification model by providing the training data set to the classification model and optimizing parameters of the classification model until the classification model produces output describing the known mutations or structural alterations.

The classification model may include a neural network, a random forest, Bayesian classifier, logistic regression, decision tree, gradient-boosted tree, multilayer perceptron, one-vs-rest, or Naive Bayes. In certain embodiments, the classification model includes a random forest, e.g., that includes at least about one thousand decision trees. The decision trees optionally receive parameters such as sample type; FASTQ quality score; alignment quality score; read coverage; or estimated probability of error. In some embodiments, the classification model includes a neural network.

The method may include testing the trained classification model on a test data set of test sequences (e.g., obtained by Sanger sequencing) that include known test mutations (e.g., SNVs) and/or chromosomal alterations. Optionally, the training data set includes a plurality of known single-nucleotide variants (SNVs), and the method includes detecting at least one SNV in the nucleic acid; validating the detected SNV as present in the nucleic acid using the classification model; and providing a report that describes the nucleic as including the structural alteration and/or the SNV.

In some embodiments, the nucleic acid from the subject is tumor DNA and the sequence reads are tumor sequence reads, and the method also includes sequencing normal DNA from the subject to produce normal sequence reads. The reference may include the normal sequence reads. Optionally, the sample includes plasma from the subject and the nucleic acid that is sequenced is cell-free DNA (cfDNA). The cfDNA may include circulating tumor DNA (ctDNA).

Where a structural alteration is found, detecting the structural alteration may include detecting at least one boundary of the structural alteration by either (a) or (b): (a) sequencing a fragment of the nucleic acid by paired-end sequencing to obtain a pair of paired-end reads, and mapping the pair of paired end reads to the reference, wherein when the pair of paired-end reads exhibit a discordant mapping to the reference, the fragment includes the boundary; and (b) sequencing the nucleic acid to determine a plurality of sequence tags, mapping the tags to the reference, and determining tag densities of mapped tags along portions of the reference, wherein when a portion of the reference exhibits an anomalous tag density a large indel is detected in a corresponding portion of the nucleic acid from the subject, wherein an end of the indel corresponds to the boundary of the structural alteration.

In other aspects, the disclosure provides a system for analyzing a tumor. The system includes a sequencing instrument for sequencing DNA from a sample from a subject to produce sequence reads, as well as a computer comprising a processor coupled to non-transitory memory.

The computer compares the sequence reads to a reference to detect a mutation or structural alteration, and validates the detected mutation or alteration as present in the DNA using a classification model. The classification model has been trained on a training data set. The training data set is a set of sequences that include known mutations or structural alterations. The system provides a report that describes the DNA from the subject as including the detected mutation or structural alteration. The system may include any suitable classification model, and it may be trained e.g., by providing the training data set to the classification model and optimizing parameters of the classification model until the classification model produces output describing the known mutation or structural alterations. Any suitable classification model may be used such as, for example, a neural network, a random forest, Bayesian classifier, logistic regression, decision tree, gradient-boosted tree, multilayer perceptron, one-vs-rest, or a Naïve Bayes operation. In certain embodiments, the classification model includes a random forest, e.g., with at least about 1,000 decision trees. The decision trees may receive parameters such as one or any combination of sample type, FASTQ quality score, alignment score, read coverage, and an estimated probability of error.

In some embodiments, the classification model includes a neural network. The neural network may be, for example, a deep-learning neural network with multiple (e.g., 5 or more) layers, which may include an input layer, a plurality of hidden layers, and an output layer. The trained classification model may optionally be tested on a test data set of test sequences (e.g., obtained by Sanger sequencing) that include known test chromosomal rearrangements. Sanger sequences for the test data may be preferred as Sanger sequencing may be understood to provide what is sometimes called a gold standard result.

In certain embodiments, the system detects and reports both structural alterations and small mutations such as single nucleotide variants (SNVs) and small indels. The training data set may include a plurality of known single-nucleotide variants (SNVs) (and/or small indels), and the method may include detecting at least one SNV in the DNA and validating the detected SNV as present in the DNA using the classification model. Preferably, the report then describes the DNA as including the SNV.

Methods and systems of the disclosure may be used for tumor/matched normal analyses. The DNA from the subject may be tumor DNA such that the sequence reads are tumor sequence reads, and the method may also include sequencing normal DNA from the subject to produce normal sequence reads. Thus, the reference may include the normal sequence reads. Thus, the structural alteration may be detected, validated, and reported in the subject's tumor DNA relative to that subjects normal, healthy DNA.

In various embodiments, a method is provided, comprising: performing whole genome sequencing using a high-throughput sequencing system on nucleic acids from a sample obtained from a subject to generate raw data, wherein the raw data is stored in a specific format in a storage medium associated with the high-throughput sequencing system or in a local storage device communicatively coupled to the high-throughput sequencing system; transmitting the raw data from the high-throughput sequencing system to a computing system, wherein the computing system comprises at least one processor and a memory, wherein the computing system is either a local server connected to the high-throughput sequencing system or a cloud-based server accessible via a network; processing the raw data by the computing system to generate candidate variants and a set of feature values for each candidate variant of the candidate variants, wherein the processing comprises: obtaining sequence reads from the raw data; filtering the sequence reads based on a predetermined protocol to generate filtered sequence reads; determining the candidate variants based on the filtered sequence reads; and determining the set of feature values for each candidate variant of the candidate variants based on the filtered sequence reads and the raw data, wherein the feature values are stored in a specific data structure; processing the feature values by the computing system using a trained machine learning model to identify somatic mutations from the candidate variants, wherein the processing comprises: loading the trained machine learning model into the memory of the computing system, allocating memory and computational resources to execute the trained machine learning model, wherein the memory allocation comprises dynamic memory management for model execution, and wherein the computational resource allocation comprises assigning processing tasks to specialized accelerators of the computing system, inputting the feature values into the trained machine learning model, and generating, by the trained machine learning model, output data comprising the somatic mutations and associated metadata; transmitting results based on the somatic mutations from the computing system to an end device, wherein the end device is communicatively connected to the computing system via the network; and displaying a report on the end device based on the results.

In some embodiments, the specific format is a .bcl file, a .cbcl file, a FASTQ file, a fast5 file, a .bax.h5 file, a .subreads.bam file, a FASTA file, a .dat file, a .bam file, or a .pod5 file.

In some embodiments, the high-throughput sequencing system generates the raw data, wherein the raw data is divided into data chunks in real time prior to transmitting to the computing system, and wherein tasks comprising base calling are offloaded to the computing system in parallel as the data chunks are received by the computing system.

In some embodiments, the nucleic acids are cell-free DNA (cfDNA) isolated from a liquid sample of the subject.

In some embodiments, the whole genome sequencing is a low-coverage whole genome sequencing (lcWGS), a medium-coverage whole genome sequencing, or a high-coverage whole genome sequencing.

In some embodiments, the specific data structure is a table, a multi-dimensional array, a linked list, a DataFrame, a JSON, a matrix, or a spreadsheet.

In some embodiments, the table is a relational database table or a hash table.

In some embodiments, the trained machine learning model is a random forest model.

In some embodiments, the random forest model comprises at least 500 decision trees.

In some embodiments, the predetermined protocol comprises filtering out all short tandem repeats (STRs) or STRs that fall outside of exons.

In some embodiments, each feature value corresponds to a predetermined feature, and wherein the set of features values corresponds to at least 20 predetermined features.

In some embodiments, the at least 20 predetermined features comprise (i) a count of unique instances where each nucleotide is observed at a specific position in the candidate variant, (ii) a count of sequence reads at a specific position in the candidate variant that show a mutated allele, (iii) a specific statistic of a quality score at a specific position in the candidate variant, (iv) a count of mutant allele pairs observed in forward strand reads at a specific position in the candidate variant, (v) a count of mutant allele pairs observed in reverse strand reads at a specific position in the candidate variant, (vi) a count of sequence reads at a specific position in the candidate variant that show a mutated allele above a cutoff quality score, and (vii) a mutation type.

In some embodiments, the candidate variants comprise at least a 100,000 variants.

In some embodiments, the method further comprises: fragmenting the nucleic acids into DNA fragments, wherein each DNA fragment is about 200-500 base pairs; ligating paired-end adapters to both ends of each DNA fragments; and amplifying the ligated DNA fragments to generate a DNA library, wherein the whole genome sequencing is paired-end sequencing on the DNA library.

In some embodiments, the method further comprises training a machine learning model to select somatic mutations from candidate variants, wherein the training comprises: obtaining training data, wherein the training data comprises sequencing data from matched tumor-normal samples, wherein the sequencing data comprises labeled variants and a set of feature values for each labeled variant, wherein each labeled variant is labeled as a somatic mutation or a non-somatic mutation; generating a training set and a validation set from the training data; inputting the training set into the machine learning model to train the machine learning model to minimize a misclassification error; validating the machine learning model using the validation set; in response to a determination that a predetermination standard is not met, tuning hyperparameters of the machine learning model and iteratively performing the inputting and the validating steps; and in response to a determination that the predetermination standard is met, storing the trained machine learning model in the local server or the cloud server.

In some embodiments, the method further comprises implementing a multi-tiered memory caching system in the computing system to optimize retrieving and processing the raw data and feature values.

In some embodiments, the multi-tiered memory caching system comprises a priority queue for candidate variants, and wherein the candidate variants are stored based on a confidence level generated by the trained machine learning model.

In some embodiments, displaying the report further comprises integrating the somatic mutations with external clinical databases and knowledge repositories to provide real-time contextual annotations and a recommendation for a personalized treatment plan.

In some embodiments, the contextual annotations include drug-gene interaction information, clinical trial eligibility, and/or prognostic insights to provide the recommendation for the personalized treatment plan.

In some embodiments, the method further comprises: determining a presence or absence of each mutation of a specific set of mutations based on the somatic mutations; and determining a genotype-directed therapy for the subject based on the presences or absences.

In some embodiments, the method further comprises evaluating the somatic mutations association with specific inherited conditions.

In some embodiments, the method further comprises: determining a ctDNA level based on the somatic mutations; and evaluating a response to a treatment based on the ctDNA level or a remission status of a disease associated with the patient.

In some embodiments, a system is provided that includes one or more processors, and a memory that is coupled to the one or more processors and stores a plurality of instructions which, when executed by the one or more processors, cause the one or more processors to perform any of the methods disclosed herein.

In some embodiments, a computer-program product is provided that is tangibly embodied in a non-transitory computer-readable memory that includes instructions which, when executed by the one or more processors, cause the one or more processors to perform any of the methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the disclosure. Thus, it should be understood that although the present application has been specifically disclosed by embodiments and optional features, modification and variation of the concepts herein disclosed may be resorted to by those skilled in the art, and that such modifications and variations are considered to be within the scope of this disclosure as defined by the appended claims.

### BRIEF DESCRIPTION OF THE DRAWINGS

The drawings illustrate certain embodiments of the technology and are not limiting. For clarity and ease of illustration, the drawings are not made to scale, and, in some instances, various aspects may be shown exaggerated or enlarged to facilitate an understanding of particular embodiments.

FIG. 1 diagrams a method for analyzing nucleic acid in accordance with various embodiments.

FIG. 2 diagrams the workflow for sequence analysis in accordance with various embodiments.

FIG. 3 illustrates detecting a translocation from mate-pair tags in accordance with various embodiments.

FIG. 4 illustrates output from use of digital karyotyping to detect an alteration boundary in accordance with various embodiments.

FIG. 5 illustrates a random forest classification model in accordance with various embodiments.

FIG. 6 illustrates a neural network in accordance with various embodiments.

FIG. 7 illustrates calling a single nucleotide variant (SNV) by read-mapping in accordance with various embodiments.

FIG. 8 shows a report produced by methods and systems of the disclosure in accordance with various embodiments.

FIG. 9 shows a system for performing methods of the disclosure in accordance with various embodiments.

FIG. 10 shows an exemplary computing environment for implementing NGS workflows in accordance with various embodiments.

FIG. 11 is an exemplary flowchart illustrating a process for a genetic screening assay to identify and display high-confidence somatic mutations in accordance with embodiments.

FIG. 12 shows a block diagram of an exemplary machine learning pipeline comprising several subsystems that work together to train, validate, and implement one or more machine learning models in accordance with various embodiments.

FIG. 13 is a diagram of a method of mutation detection in accordance with various embodiments.

FIG. 14 shows positive predictive value vs. sensitivity for simulated low-purity tumor datasets created from normal cell line sequence data in accordance with various embodiments.

FIG. 15 shows sensitivity stratified by mutation type, calculated from simulated mutations across mutation types and allele frequencies in accordance with various embodiments.

FIG. 16 gives positive predictive value vs. sensitivity for cell line datasets in accordance with various embodiments.

FIG. 17 shows mutational loads between two mutation calls in accordance with various embodiments.

FIG. 18 reports unique/shared status for somatic mutations across samples in accordance with various embodiments.

FIG. 19 shows evaluation of various genes in accordance with various embodiments.

FIG. 20 is a distribution of problematic TCGA driver gene calls by genes with approved FDA therapies (left panel) or ongoing clinical trials (right panel) in accordance with various embodiments.

FIG. 21 gives Cerebro confidence scores in accordance with various embodiments.

FIG. 22 shows the unique/shared mutation status for all patients in accordance with various embodiments.

FIG. 23 reports problematic mutations annotated by characteristic issue in accordance with various embodiments.

FIG. 24 is a Kaplan-Meier analysis of progression free survival (left) or overall survival (right) using tumor mutation loads from original publications in accordance with various embodiments.

FIG. 25 is a Kaplan-Meier analysis of same samples using Cerebro mutational loads Log-rank P-value shown for each survival plot in accordance with various embodiments.

FIG. 26 is Table 1 showing whole exome sequencing analysis and somatic mutations loads in accordance with various embodiments.

FIG. 27 is Table 2 showing a comparison of NGS clinical sequencing in accordance with various embodiments.

FIGS. 28A-28F show false position evaluation for somatic mutation callers in accordance with various embodiments.

FIG. 29 illustrates the results of droplet digital PCR mutation validity analyses in accordance with various embodiments.

FIGS. 30A and 30B illustrate curve analyses for Cerebro and other mutation callers using experimentally validated alterations in accordance with various embodiments.

FIG. 31 illustrates mutation loads for TCGA exomes using different mutation calling methods in accordance with various embodiments.

FIG. 32 illustrates concordance rates of Cerebro compared to other mutation call sets for TCGA exomes in accordance with various embodiments.

FIGS. 33A-33D illustrate comparison of Cerebro mutation calls with published calls in accordance with various embodiments.

FIG. 34 illustrates comparative results of three clinical sequencing panels in accordance with various embodiments.

FIGS. 35A-35C illustrate alterations confirmed by ddPCR in a clinical panel comparison in accordance with various embodiments.

### TERMS

As used herein, the articles “a” and “an” are used herein to refer to one or to more than one (i.e. at least one) of the grammatical object of the article. By way of example, an element means at least one element and can include more than one element.

As used herein, the terms “about,” “substantially,” and “approximately” are defined as being largely but not necessarily wholly what is specified (and include wholly what is specified) as understood by one of ordinary skill in the art. In any disclosed embodiment, the term “about,” “substantially,” or “approximately” may be substituted with “within [a percentage] of” what is specified, where the percentage includes 0.1 percent, 1 percent, 5 percent, 10 percent, 20 percent, etc. Moreover, the term terms “about,” “substantially,” and “approximately” are used to provide flexibility to a numerical range endpoint by providing that a given value may be slightly above or slightly below the endpoint without affecting the desired result. In some embodiments, the term “about” refers to a ±5% to ±10% of the stated value, for example, “about 100” means a range of 90 to 110.

As used herein, the terms “aligned,” “alignment,” and “aligning” refer to two or more nucleic acid sequences that can be identified as a match (e.g., 100% identity) or partial match (e.g., less than 100% identity).

As used herein, the term “allele” refers to any alternative forms of a gene at a particular locus. There may be one or more alternative forms, all of which may relate to one trait or characteristic at the specific locus. In a diploid cell of an organism, alleles of a given gene can be located at a specific location, or locus (loci plural) on a chromosome. The genetic sequences that differ between different alleles at each locus are termed “variants,” “polymorphisms,” or “mutations.” The term “single nucleotide polymorphisms” (SNPs) can be used interchangeably with “single nucleotide variants” (SNVs).

As used herein, the term “and/or” refers to and encompasses any and all possible combinations of one or more of the associated listed items, as well as the lack of combinations were interpreted in the alternative (“or”).

As used herein, when an action is “based on” something, this means the action can be based at least in part on at least a part of the something.

As used herein, the term “cell-free nucleic acid” or “CFNA” refers to extracellular nucleic acids, as well as circulating free nucleic acid. As such, the terms “extracellular nucleic acid,” “cell-free nucleic acid” and “circulating free nucleic acid” are used interchangeably. Extracellular nucleic acids can be found in biological sources such as blood, urine, stool, and other fluids including saliva, cerebrospinal fluid, surgical drain fluid, and cyst fluid. CFNA may refer to cell-free DNA (cfDNA), circulating free DNA (cfDNA), cell-free RNA (cfRNA), or circulating free RNA (cfRNA).

As used herein, the term “exon” refers to a coding region of a gene that contains information required for synthesizing proteins, with exceptions that some exons may contribute to untranslated regions (UTRs) of RNA. For example, the human genome contains approximately 20,000 protein-coding genes, with the number of exons per gene varying widely. The average human gene contains about 8-12 exons, but some genes, such as the Titin gene, can have over 300 exons. Similarly, the lengths of individual exons vary significantly, ranging from less than 50 base pairs (bp) to over 1,000 bp. For instance, the majority of human exons has a median length of approximately 150 bp, while some exons involved in UTRs or regulatory functions may be longer.

As used herein, the term “genetic screening” refers to a process of testing individuals or populations for specific genetic traits, mutations, or abnormalities that may indicate a predisposition to certain diseases, conditions, or inherited disorders, including but not limited to the followings: prenatal genetic screening tests (e.g., non-invasive prenatal testing (NIPT) or non-invasive prenatal screening (NIPS), first trimester screening, second trimester screening (quad screen), carrier screening, amniocentesis, and chorionic villus sampling (CVS)); newborn screening tests (e.g., the heel prick test (Guthrie test)); cancer genetic screening (e.g., BRCA1 and BRCA2 testing, Lynch syndrome screening, and FAP (familial adenomatous polyposis) testing); cardiovascular genetic screening (e.g., familial hypercholesterolemia testing and hypertrophic cardiomyopathy testing); neurological genetic screening (e.g., Huntington's disease testing and Alzheimer's disease genetic testing); metabolic and other genetic disorders screening (e.g., cystic fibrosis testing, thalassemia and sickle cell disease testing, and hemochromatosis testing); pharmacogenomic testing includes (e.g., cytochrome P450 testing); ancestry and health-related genetic screening; rare disease screening (e.g., exome sequencing and whole genome sequencing); genetic screening for specific populations; carrier screening; and prenatal and preconception screening (e.g., expanded carrier screening).

As used herein, the term “likely” refers to a probability range of about 80%-99% when describing the significance of an event. In some embodiments, “likely” is 95%-98%. For example, a “likely benign” variant has a 95%-98% chance of being benign, and a “likely pathogenic” variant has a 95%-98% chance of being pathogenic. Different ranges may be used for different events.

As used herein, the term “low-coverage whole genome sequencing,” “low-coverage WGS,” or “lcWGS” refers to a sequencing approach in which the entire genome of an organism is sequenced at a relatively low depth of coverage, typically ranging from 1× to 10×, meaning that each base in the genome is sequenced an average of 1 to 10 times. In some instances, lcWGS includes ultra-low-coverage WGS (ulcWGS), where the sequencing is performed at an even lower depth of coverage, typically below 1× (e.g., 0.1× to 1×).

As used herein, the term “mutant” or “variant,” when made in reference to an allele or sequence, generally refers to an allele or sequence that does not encode the phenotype most common in a particular natural population. The terms “mutant allele” and “variant allele” can be used interchangeably. In some cases, a mutant allele can refer to an allele present at a lower frequency in a population relative to the wild-type allele. Mutant alleles may be inherited or acquired. In some cases, a mutant allele or sequence can refer to an allele or sequence mutated from a wild-type sequence to a mutated sequence that presents a phenotype associated with a disease state and/or drug-resistant state. Mutant alleles and sequences may be different from wild-type alleles and sequences by only one base but can be different up to several bases or more. The term “mutant” when made in reference to a gene generally refers to one or more sequence mutations in a gene, including a point mutation, a SNP, an insertion, a deletion, a substitution, a transposition, a translocation, a copy number variation, or another genetic mutation, alteration, or sequence variation. In some instances, the term “mutation” is used interchangeably with “alteration” or “variant.”

As used herein, the term “nucleic acid” and “nucleic acids” refer to deoxyribonucleic acids (DNA) or ribonucleic acids (RNA) and polymers thereof in either single- or double-stranded form. Unless specifically limited, the term encompasses nucleic acids containing known analogues of natural nucleotides that have comparable properties as the reference nucleic acid. A nucleic acid sequence can comprise combinations of deoxyribonucleic acids and ribonucleic acids. Such deoxyribonucleic acids and ribonucleic acids include both naturally occurring molecules and synthetic analogues. Nucleic acids also encompass all forms of sequences including, but not limited to, single-stranded forms, double-stranded forms, hairpins, stem-and-loop structures, and the like.

As used herein, the terms “patient” and “subject” are used interchangeably and refer to a mammal, including humans and non-human primates, regardless of age. The subject may be healthy, suspected of having a disease, diagnosed with a disease, or undergoing treatment for a disease. For example, the subject could be healthy, suspected of having cancer, diagnosed with cancer, or currently receiving treatment for cancer. Subjects also encompass living humans who are receiving medical care for a disease or condition, as well as individuals without a defined illness who are undergoing evaluation for signs or symptoms of disease. In some embodiments, the term “patient” or “subject” also encompasses a pregnant woman, including those undergoing routine prenatal care, non-invasive testing, or evaluations for complications during pregnancy.

As used herein, the term “read” or “sequence read” is a short nucleotide sequence produced by any sequencing process, including NGS, described herein or known in the art.

As used herein, the term “reference genome” can refer to any particular known, sequenced or characterized genome, whether partial or complete, of any organism or virus which may be used to reference identified sequences from a subject.

As used herein, the term “sample,” “biological sample,” “tissue,” or “tissue sample” refers to any sample including a biomolecule (such as a protein, a peptide, a nucleic acid, a lipid, a carbohydrate, or a combination thereof) that is obtained from any organism including viruses. Other examples of organisms include mammals (such as humans; veterinary animals like cats, dogs, horses, cattle, and swine; and laboratory animals like mice, rats and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

As used herein, the term “standard” or “reference” refers to a substance which is prepared to certain pre-defined criteria and can be used to assess certain aspects of, for example, an assay. Standards or references preferably yield reproducible, consistent, and reliable results. These aspects may include performance metrics, examples of which include, but are not limited to, accuracy, specificity, sensitivity, linearity, reproducibility, limit of detection and/or limit of quantitation. Standards or references may be used for assay development, assay validation, and/or assay optimization. Standards may be used to evaluate quantitative and qualitative aspects of an assay. In some instances, applications may include monitoring, comparing and/or otherwise assessing a QC sample/control, an assay control (product), a filler sample, a training sample, and/or lot-to-lot performance for a given assay.

As used herein, the term “segment” or “genomic segment” refers to one or more genomic portions, and often includes one or more consecutive portions (e.g., about 2 to about 100 such portions (e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90 such portions)). A segment or genomic segment is a part of the target chromosome, gene, exon, intron or other region of interest. In some instances, a segment can include non-consecutive portions. The term “segment” may be used interchangeably with “region of interest,” “ROI,” or “target region.” In some embodiments, a segment may refer to a single nucleotide, a sequence of multiple nucleotides, a single exon, an entire gene, a chromosomal arm, or any other defined region of genetic material.

As used herein, the term “sequence variant” refers to any variation in sequence relative to one or more reference sequences. A sequence variant may occur with a lower frequency than the reference sequence for a given population of individuals for whom the reference sequence is known. In some cases, the reference sequence is a single known reference sequence, such as the genomic sequence of a single individual. In some cases, the reference sequence is a consensus sequence formed by aligning multiple known sequences, such as the genomic sequence of multiple individuals serving as a reference population, or multiple sequencing reads of polynucleotides from the same individual. In some cases, the sequence variant occurs with a low frequency in the population (also referred to as a “rare” sequence variant). For example, in non-tissue samples, the sequence variant may occur with a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. In some non-tissue sample cases, the sequence variant occurs with a frequency of about or less than about 0.1%. In tissue, the sequence variant may occur with a frequency of about or less than about 100%, 90%, 80%, 70%, 60%, 50%, 40%, 30%, 20%, 10%, 5%, or lower. A sequence variant can be any sequence that varies from a reference sequence. A sequence variation may consist of a change in, insertion of, or deletion of a single nucleotide, or of a plurality of nucleotides (e.g., 2, 3, 4, 5, 6, 7, 8, 9, 10, or more nucleotides). Where a sequence variant includes two or more nucleotide differences, the nucleotides that are different may be contiguous with one another, or discontinuous. Non-limiting examples of types of sequence variants include single nucleotide polymorphisms (SNP), deletion/insertion polymorphisms (INDEL), copy number variants (CNV), loss of heterozygosity (LOH), microsatellite instability (MSI), variable number of tandem repeats (VNTR), and retrotransposon-based insertion polymorphisms. Additional examples of types of sequence variants include those that occur within short tandem repeats (STR) and simple sequence repeats (SSR), or those occurring due to amplified fragment length polymorphisms (AFLP) or differences in epigenetic marks that can be detected (e.g., methylation differences). In some instances, a sequence variant can refer to a chromosome rearrangement, including but not limited to a translocation or fusion gene, or rearrangement of multiple genes resulting from, for example, chromothripsis. As used herein, the term “reference genome” can refer to any particular known, sequenced or characterized genome, whether partial or complete, of any organism or virus which may be used to reference identified sequences from a subject.

Unless defined otherwise, all technical and scientific terms used herein have the same meaning as commonly understood by one of ordinary skill in the art to which this disclosure belongs. Although any methods and materials similar to or equivalent to those described herein can be used in the practice or testing of the application, the preferred methods and materials are now described.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### I. Introduction

Advancements in genomic technologies, particularly Next-Generation Sequencing (NGS) methods such as Whole Genome Sequencing (WGS) and Whole Exome Sequencing (WES), have revolutionized the analysis of complex genetic information. These techniques are now indispensable in research, clinical diagnostics, and personalized medicine. WGS offers a comprehensive dataset by sequencing the entire genome, including both coding and non-coding regions, enabling the detection of a wide range of genetic alterations, such as single nucleotide variants (SNVs), insertions, deletions, structural rearrangements, and somatic mutations associated with diseases like cancer. In contrast, WES focuses specifically on the protein-coding regions of the genome (exons), which constitute only 1-2% of the genome but contain the majority of disease-causing mutations. In oncology, these NGS methods are increasingly utilized for molecular profiling, which supports diagnostic and therapeutic decision-making. Tumor-specific somatic mutations—genetic alterations acquired during an individual's lifetime—can disrupt key driver genes involved in tumorigenesis and lead to the accumulation of additional mutations over time. These changes influence tumor biology and present new opportunities for therapeutic intervention.

Despite the transformative potential of WGS and WES, conventional methods for processing and analyzing the data they produce encounter significant challenges that limit their accuracy, efficiency, and clinical applicability. A primary obstacle stems from the sheer volume of data generated. WGS and WES produce millions of short sequence reads, typically ranging from 50 to 300 base pairs in length, which must be computationally assembled to reconstruct the genome or exome. This assembly process becomes particularly complex in genomic regions characterized by repetitive sequences, high GC content, or structural variations. In such regions, short reads often fail to align accurately or resolve ambiguities, resulting in gaps or errors in the reconstructed sequences. These challenges are further amplified when detecting somatic mutations, especially those with low allelic frequencies. In heterogeneous tumor samples, where mutant cells coexist with normal cells, identifying these low-frequency variants requires ultra-deep sequencing and advanced bioinformatics tools capable of distinguishing true somatic mutations from sequencing noise and artifacts.

Traditional methods for detecting somatic mutations exacerbate these challenges by relying heavily on predefined thresholds and rigid filtering criteria. Although these approaches aim to minimize false positives, they often exclude clinically significant mutations or fail to identify subtle yet critical genetic alterations, ultimately compromising both the sensitivity and specificity of the analysis. Additionally, the vast datasets generated by WGS and WES-often reaching hundreds of gigabytes per sample-place immense demands on computational resources. The initial stages of assembling short reads into meaningful genomic sequences are particularly computationally intensive, especially in regions with repetitive elements or low-quality sequencing data. These difficulties cascade into downstream analyses, further limiting the overall effectiveness and utility of traditional workflows.

Another challenge lies in the quality of the input samples used for sequencing. Samples derived from formalin-fixed paraffin-embedded (FFPE) tissues or cell-free DNA (cfDNA) from liquid biopsies often present unique obstacles. DNA extracted from FFPE samples is frequently degraded, chemically modified, or contaminated with non-target nucleic acids, making it difficult to generate high-quality sequencing libraries. Similarly, cfDNA, which is present in low abundance in the bloodstream, is often overwhelmed by background DNA from normal cells, complicating the detection of tumor-specific somatic mutations. These issues further reduce the sensitivity and specificity of NGS workflows, particularly in clinical applications where accurate mutation detection is critical.

Together, these challenges highlight the limitations of conventional methods for processing and analyzing WGS and WES data, particularly in the context of detecting clinically relevant somatic mutations. To address these challenges, there is an urgent need for innovative solutions that enhance mutation detection accuracy and deliver reliable, actionable results. Advances in computational technologies, such as machine learning, optimized data processing pipelines, and novel approaches to library preparation and sequencing data analysis, hold significant promise in overcoming these obstacles. By addressing these limitations, it will be possible to maximize the clinical and research potential of WGS and WES, ultimately improving outcomes in precision medicine and genomic research.

More specifically, to address these challenges and others, techniques disclosed herein integrate machine learning techniques into the NGS workflow to improve the detection and classification of somatic mutations from both WGS and WES data. These WES and WGS workflows introduce significant computational advancements, including the integration of optimized data pipelines, automated machine learning workflows, and enhanced data processing infrastructures. By leveraging these improvements, the techniques disclosed herein ensure efficient handling of the massive data volumes generated by WES and WGS, minimizes errors in mutation detection, and supports the delivery of actionable insights for clinical and research applications. These techniques not only improve the accuracy and sensitivity of somatic mutation detection but also enable real-time processing and reporting, facilitating faster and more informed decision-making in precision medicine and personalized treatment strategies.

For WES, the process begins with the amplification of cfDNA extracted from a patient sample to generate multiple copies of nucleic acid sequences, followed by sequencing the exome to identify candidate variants. The method analyzes features associated with the sequencing and variant-calling processes to generate a set of feature values for each candidate variant. These feature values are then processed using a trained machine learning classifier, such as a random forest model. Each decision tree of the random forest model evaluates a unique combination of selected information supporting the candidate variant and classifies the variant as somatic or not somatic. The model generates a confidence score, representing the proportion of decision trees that classify the variant as somatic, which forms the basis for the final classification. A report describing the somatic mutations is then generated and displayed for use by clinicians in diagnostic or therapeutic decision-making.

For WGS, the process begins with sequencing a sample using a high-throughput sequencing system (e.g., a high-throughput sequencer) to generate raw sequencing data, which is subsequently stored in a specific format within the sequencer's local storage or transmitted to a local server (on-premises server) or a cloud-based server for analysis. The raw sequencing data undergoes initial computational processing using predetermined protocols to filter sequence reads, ensuring that only high-quality data is retained for downstream analysis. Advanced algorithms are employed to identify candidate variants from the filtered sequence reads, followed by feature extraction to generate a comprehensive set of feature values for each candidate variant. These feature values are stored in an optimized data structure, enabling efficient access and processing by downstream computational models. The extracted feature values are then analyzed using a trained machine learning model, such as a random forest classifier, which evaluates the data to accurately select somatic mutations from the candidate pool. The somatic mutations are transmitted from the local server or the cloud server to an end device for generating and displaying a report on the end device based on the somatic mutations.

The integration of machine learning into WGS and WES workflows offers several significant advantages over conventional approaches. By leveraging the power of machine learning, the method improves the sensitivity and specificity of somatic mutation detection, particularly for low-frequency variants or in challenging sample types like cfDNA, where traditional filtering thresholds may fail to differentiate true mutations from noise. More specifically, unlike conventional algorithms, machine learning models (e.g., random forest classifiers) are trained to recognize subtle patterns in complex datasets, leading to fewer false positives and false negatives when identifying somatic mutations. Machine learning models can recognize subtle patterns and classify somatic mutations with greater precision by learning from labeled datasets, enabling better distinction between real mutations and sequencing artifacts. This is particularly beneficial in regions with repetitive elements or poor sequencing quality. For example, the disclosed method enables the identification of somatic mutations with a high degree of accuracy, as evidenced by experimentally validated tumor alterations detected with a sensitivity of 97% and a positive predictive value of 98%, outperforming many existing tools. Additionally, machine learning models can generalize across different datasets, sequencing platforms, and error profiles, making them more robust compared to static, rule-based conventional methods. Lastly, machine learning enables the integration of multiple feature types (e.g., read depth, base quality, mapping quality) into a single predictive model, capturing more nuanced relationships that conventional methods may overlook.

The integration of machine learning techniques not only enhances the analysis itself but also optimizes the computing systems used for WGS and WES workflows in several key ways. Firstly, there is efficient use of computational resources by the computing system. Machine learning-based filtering protocols ensure that only high-quality sequence reads are retained for downstream analysis, reducing the size of the dataset and computational burden compared to conventional methods that process larger volumes of raw data. By focusing computational efforts on “candidate variants” identified early in the process, the machine learning techniques minimize unnecessary processing of irrelevant data. Also, the use of optimized data structures for storing feature values (e.g., indexed or compressed formats) allows for faster data retrieval and reduced memory overhead during analysis. This contrasts with conventional methods that often rely on less efficient or bulkier data representations for ruled based processing. Additionally, the machine learning models, such as random forest classifiers or deep learning frameworks, are inherently parallelizable. These models leverage modern computing architectures, such as GPUs and cloud-based computing clusters, to accelerate the analysis process. Conventional methods, which often rely on single-threaded or CPU-based workflows, struggle to scale with the growing size of WGS/WES datasets.

Secondly, there is reduced computational complexity, and thus less computing resources required by the computing system to run the algorithm and execute the processes. Instead of assembling all short reads into complete genomic sequences (a highly computationally intensive task), machine learning techniques focus on extracting meaningful features directly from sequence reads. This reduces the computational complexity of variant calling workflows. Conventional methods often require full alignment and assembly steps, which are time-consuming and resource-intensive, especially in regions with poor sequencing quality. Additionally, by training machine learning models to prioritize high-confidence variants, fewer computational resources are wasted on low-quality candidate variants. This contrasts with conventional methods, which may require additional post-processing steps and resources (e.g., CPU cycles and memory) to filter out false positives.

Thirdly, there is enhanced processing speed, and thus shorter processing times by the computing system. Machine learning models are capable of processing data in real time or near-real time, as they rely on pre-trained algorithms that evaluate candidate variants quickly. Conventional methods often require multiple iterative steps for read alignment, assembly, and variant calling, leading to longer processing times. Additionally, machine learning automates tasks like variant prioritization and classification, reducing the need for manual intervention. This not only speeds up the workflow but also reduces the risk of human error.

Fourthly, there is improved storage use and data management within and by the computing system. Machine learning workflows focus on extracting and storing only the most relevant features for each candidate variant, leading to smaller intermediate datasets that are easier to store and manage. Conventional methods, which often process and store large volumes of raw data or intermediate assembly outputs, require significantly more storage space. Additionally, machine learning models are well-suited for cloud-based implementations, where computational resources can be dynamically allocated based on the complexity of the dataset. This allows for cost-effective scaling and optimization of computing resources. Conventional methods such as rigid rule-based systems often face challenges in adapting to cloud environments due to their reliance on static workflows and limited parallelization.

Additionally, the disclosed techniques enable efficient data flow and processing using sequencers and servers. Sequencers generate vast amounts of raw data in real time, which can be immediately divided into manageable chunks to facilitate faster transfer. These data chunks are streamed from the sequencer to a network of servers using high-speed data transfer protocols (e.g., FTP, Aspera, or Rsync) to prevent delays caused by bandwidth limitations. To further enhance efficiency, tasks such as base calling or quality filtering are offloaded to servers in parallel as the data arrives, reducing latency between data generation and preprocessing. Real-time monitoring systems can also be used to track transfer speeds, server capacity, and task progress, allowing for proactive adjustments, such as rerouting data to less-burdened servers or addressing network interruptions immediately. Conventional methods often stores raw sequencing data temporarily on local storage devices before being transferred to servers for processing, creating delays and risks of storage overload. Conventional workflows also process data in a step-by-step manner, waiting for the complete dataset to be generated before initiating preprocessing tasks, leading to significant idle time. In contrast, the disclosed techniques provide seamless integration of sequencers and servers, ensuring that bottlenecks in data generation, transfer, and processing are minimized, enabling high-throughput sequencing workflows to operate smoothly and scale effectively.

Lastly, the computing system has improved energy efficiency. By reducing the overall computational workload (e.g., through data filtering, feature extraction, and focused analysis), machine learning-based workflows consume less energy per analysis compared to conventional rule-based methods. This is particularly important in large-scale genomic studies (WGS and WES), where the cumulative energy demands of conventional workflows can be substantial.

The integration of machine learning techniques into the WGS and WES workflows as described herein also improves robustness and error handling of the computing system. The machine learning models are trained to account for sequencing errors, low-quality reads, and other noise in the data, reducing the need for computationally expensive error correction steps. Conventional methods often require additional pre-processing steps to handle noise, increasing computational demands. Additionally, the machine learning workflows can tolerate interruptions or hardware failures by resuming from intermediate steps, particularly in distributed computing environments. Conventional workflows, by contrast, may need to restart from earlier steps, wasting time and computing resources.

Moreover, the WGS and WES workflows are flexible enough to be scaled for large-scale studies and diagnostic testing. Machine learning models can process large batches of data simultaneously, making them ideal for high-throughput genomic studies. Conventional methods, which often rely on sequential processing steps, are less suited for such large-scale analyses. Additionally, machine learning-based workflows can easily integrate with distributed computing systems, allowing for efficient scaling across multiple nodes or cloud servers. Conventional methods may require significant re-engineering to achieve similar scalability.

Consequently, incorporating machine learning techniques into WGS and WES workflows, not only is the detection and classification of somatic mutations improved, but the underlying computing system and functionality thereof also becomes significantly improved. The machine learning-based approaches described herein reduce computational complexity, optimize resource usage, enhance processing speed, and improve data management compared to conventional methods. These improvements make the integration of machine learning techniques into the WGS and WES workflows an important tool for addressing the challenges posed by the growing scale and complexity of genomic data.

### II. Embodiments Related to Next Generation Sequencing

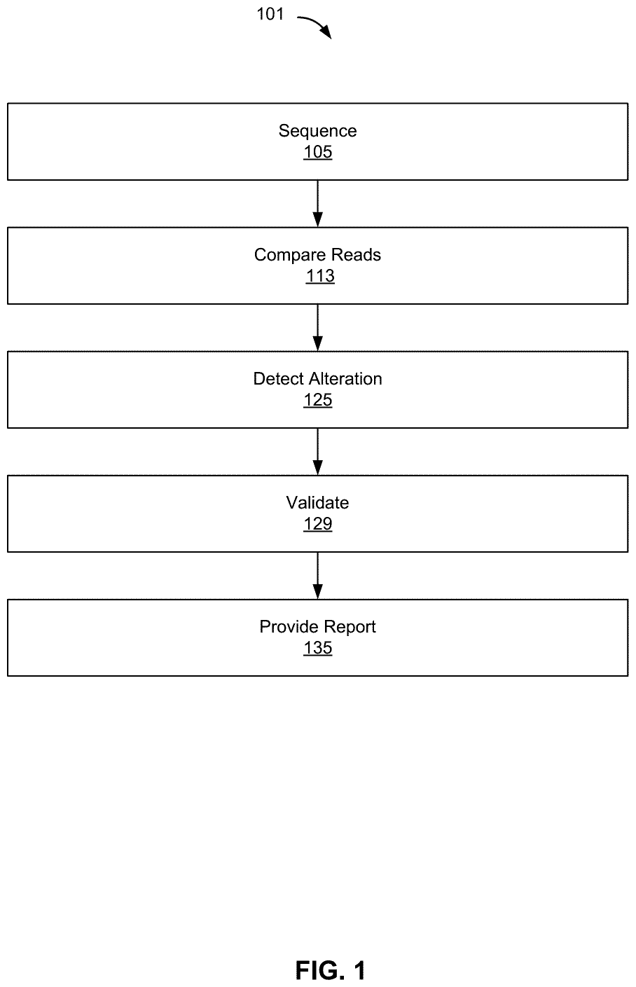

FIG. 1 diagrams a method 101 for analyzing nucleic acid. The method 101 includes sequencing 105 nucleic acid from a sample from a subject to produce sequence reads and comparing 113 the sequence reads to a reference to detect 125 at least one mutation or boundary of a structural alteration. The method 101 further includes validating 129 the mutation or detected boundary as present in the nucleic acid using a classification model trained on a training data set of sequences that include known mutations and/or structural alterations. A report is provided 135 that describes the nucleic acid from the subject as including the mutation or structural alteration.

Systems and methods disclosed herein may be used for biomarker discovery, NGS research and development, to identify biomarker targets for a drug discovery pipeline, or to provide larger, multi-analyte panels in tissue and plasma including whole exome sequencing, whole genome sequencing, and neoantigen prediction. Systems and methods may be used clinically, for tumor analysis, or may have applications for clinical trial services, e.g., to prospectively stratify patients for clinical trials. Systems and methods of the disclosure may be deployed in a decentralized oncology testing system and may be deployed to beneficially use a pre-existing installed base of sequencing technology. Systems and methods may have particular applicability for in vitro diagnostic (IVD) pipelines and particularly for reporting mutations and rearrangements in panels of genes known to be associated with cancer.

In some embodiments, methods disclosed herein are implemented via a system of components that may optionally include one or more of a sample kit, sequencing tools, and analysis tools. For examples, an assay may start with extracted DNA from tissue or plasma, which may be extracted using a reagent kit (e.g., tubes and DNA extraction reagents, etc.). The kit may include tools for library preparation such as probes for hybrid capture as well as any useful reagents & protocols for fragmentation, adapter ligation, purification/isolation, etc. Such kits may have particular applicability in IVD applications. Using kits or other techniques known in the art a sample containing DNA is obtained.

For method 101, the sample that includes nucleic acid may be obtained 105 by any suitable method. The sample may be obtained from a tissue or body fluid that is obtained in any clinically acceptable manner. Body fluids may include mucous, blood, plasma, serum, serum derivatives, bile, blood, maternal blood, phlegm, saliva, sweat, amniotic fluid, menstrual fluid, mammary fluid, follicular fluid of the ovary, fallopian tube fluid, peritoneal fluid, urine, and cerebrospinal fluid (CSF), such as lumbar or ventricular CSF. A sample may also be a fine needle aspirate or biopsied tissue. Samples of particular interest include sputum and stool, where target nucleic acid may be severely degraded, or present in only very small amounts. A sample also may be media containing cells or biological material. Samples may also be obtained from the environment (e.g., air, agricultural, water and soil) or may include research samples (e.g., products of a nucleic acid amplification reaction, or purified genomic DNA, RNA, proteins, etc.). In preferred embodiments, a sample is a blood or plasma sample from a patient.

In some embodiments, methods disclosed herein are used in the analysis of circulating tumor DNA (ctDNA). Target ctDNA may be obtained by any suitable methods. In some embodiments, cell-free DNA is captured by hybrid capture. Exemplary hybrid capture based ctDNA workflow may include sample preparation, sequencing, and analysis. For sample prep, cell-free DNA may optionally be fragmented and barcoded to create a cell-free DNA library.

In certain embodiments, the method 101 includes obtaining 105 sequencing data from nucleic acid obtained from a tumor sample and a normal sample from the same patient. The tumor sample may be a biopsy specimen, or may be obtained as circulating tumor DNA (ctDNA). The normal sample can be any bodily tissue or fluid containing nucleic acid that is considered to be cancer-free, such as lymphocytes, saliva, buccal cells, or other tissues and fluids.

Tumor samples may include, for example, cell-free nucleic acid (including DNA or RNA) or nucleic acid isolated from a tumor tissue sample such as biopsied tissue or formalin fixed paraffin embedded tissue (FFPE). Normal samples, in certain aspects, may include nucleic acid isolated from any non-tumor tissue of the patient, including, for example, patient lymphocytes or cells obtained via buccal swab. Cell-free nucleic acids may be fragments of DNA or ribonucleic acid (RNA) which are present in the blood stream of a patient. In a preferred embodiment, the circulating cell-free nucleic acid is one or more fragments of DNA obtained from the plasma or serum of the patient.

The cell-free nucleic acid may be isolated according to techniques known in the art and may include, for example: the QIAmp system from Qiagen (Venlo, Netherlands); the Triton/Heat/Phenol protocol (THP); a blunt-end ligation-mediated whole genome amplification (BL-WGA); or the NucleoSpin system from Macherey-Nagel, GmbH & Co. KG (Duren, Germany). See Xue, 2009, Optimizing the yield and utility of circulating cell-free DNA from plasma and serum, Clin Chim Acta 404 (2): 100-104, and Li, 2006, Whole genome amplification of plasma-circulating DNA enables expanded screening for allelic imbalances in plasma, J Mol Diag 8 (1): 22-30, both incorporated by reference. In an exemplary embodiment, a blood sample is obtained from the patient and the plasma is isolated by centrifugation. The circulating cell-free nucleic acid may then be isolated by any of the techniques above.

According to certain embodiments, nucleic acid may be extracted from tumor or non-tumor patient tissues. After tissue or cells have been obtained from the patient, it is preferable to lyse cells in order to isolate nucleic acids. Lysing methods are known in the art and may include sonication, freezing, boiling, exposure to detergents, or exposure to alkali or acidic conditions.

When there is an insufficient amount of nucleic acid for analysis, a common technique used to increase the amount by amplifying the nucleic acid. Amplification refers to production of additional copies of a nucleic acid sequence and is generally carried out using polymerase chain reaction or other technologies well known in the art (e.g., Dieffenbach and Dveksler, PCR Primer, a Laboratory Manual, 1995, Cold Spring Harbor Press, Plainview, NY).

Polymerase chain reaction (PCR) refers to methods by K. B. Mullis (U.S. Pat. Nos. 4,683,195 and 4,683,202, hereby incorporated by reference) for increasing concentration of a segment of a target sequence in a mixture of genomic DNA without cloning or purification.

Primers can be prepared by a variety of methods including but not limited to cloning of appropriate sequences and direct chemical synthesis using methods well known in the art (Narang et al., Methods Enzymol., 68:90 (1979); Brown et al., Methods Enzymol., 68:109 (1979)). Primers can also be obtained from commercial sources such as Operon Technologies, Amersham Pharmacia Biotech, Sigma, and Life Technologies. The primers can have an identical melting temperature. The lengths of the primers can be extended or shortened at 5′ end or 3′ end to produce primers with desired melting temperatures. Also, the annealing position of each primer pair can be designed such that the sequence and length of the primer pairs yield the desired melting temperature. The simplest equation for determining the melting temperature of primers smaller than 25 base pairs is the Wallace Rule (Td=2 (A+T)+4 (G+C)). Computer programs can also be used to design primers, including but not limited to Array Designer Software from Arrayit Corporation (Sunnyvale, CA), Oligonucleotide Probe Sequence Design Software for Genetic Analysis from Olympus Optical Co., Ltd. (Tokyo, Japan), NetPrimer, and DNAsis Max v3.0 from Hitachi Solutions America, Ltd. (South San Francisco, CA). The melting temperature of each primer is calculated using software programs such as OligoAnalyzer 3.1, available on the web site of Integrated DNA Technologies, Inc. (Coralville, IA).

Amplification and/or sequencing adapters may be attached to the fragmented nucleic acid. Adapters may be commercially obtained, such as from Integrated DNA Technologies (Coralville, IA). In certain embodiments, the adapter sequences are attached to the template nucleic acid molecule with an enzyme. The enzyme may be a ligase or a polymerase. The ligase may be any enzyme capable of ligating an oligonucleotide (RNA or DNA) to the template nucleic acid molecule. Suitable ligases include T4 DNA ligase and T4 RNA ligase, available commercially from New England Biolabs (Ipswich, MA). Methods for using ligases are well known in the art. The polymerase may be any enzyme capable of adding nucleotides to the 3′ and the 5′ terminus of template nucleic acid molecules.

The input sample that is sequenced 105 may include entire genomes, chromosomes, or genes, or at least substantial portions thereof. A whole-genome assay might be desirable where the patient has an unknown cancer, and a broad approach is necessary to pinpoint the mutations present. When tumor nucleic acid is isolated from ctDNA, and the type or location of the tumor is otherwise unknown, it may be desirable to analyze the whole genome. The mutations in the ctDNA can potentially include mutations from many tumors in the body, so performing a broad analysis on ctDNA will give a more complete picture of the progression of cancer in the body.

In some embodiments, a panel (e.g., tens or hundreds) of known cancer-related genes may be assayed. A panel may cover a range of genes of biological and clinical importance in human cancer. Some of the types of cancer covered by this panel are breast cancer, colorectal cancer, leukemia, prostate cancer and lymphoma. Assaying a whole genome or a panel of genes may include screening for alterations such as copy number variation, translocations, large indels, or inversions.

The nucleic acids can be sequenced 105 using any sequencing platform known in the art. Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLID sequencing. Sequencing of separated molecules has more recently been demonstrated by sequential or single extension reactions using polymerases or ligases as well as by single or sequential differential hybridizations with libraries of probes.

A sequencing technique that can be used includes, for example, use of sequencing-by-synthesis systems sold under the trademarks GS JUNIOR, GS FLX+ and 454 SEQUENCING by 454 Life Sciences, a Roche company (Branford, CT), and described by Margulies, M. et al., Genome sequencing in micro-fabricated high-density picotiter reactors, Nature, 437:376-380 (2005); U.S. Pat. Nos. 5,583,024; 5,674,713; and 5,700,673, the contents of which are incorporated by reference herein in their entirety. 454 sequencing involves two steps. In the first step of those systems, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized).

Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP-by-ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used is SOLID technology by Applied Biosystems from Life Technologies Corporation (Carlsbad, CA). In SOLID sequencing, genomic DNA is sheared into fragments, and adaptors are attached to the 5′ and 3′ ends of the fragments to generate a fragment library. Alternatively, internal adaptors can be introduced by ligating adaptors to 5′ and 3′ ends of the fragments, circularizing the fragments, digesting the circularized fragment to generate an internal adaptor, and attaching adaptors to 5′ and 3′ ends of the resulting fragments to generate a mate-paired library. Next, clonal bead populations are prepared in microreactors containing beads, primers, template, and PCR components. Following PCR, the templates are denatured, and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is removed, and the process is then repeated.

Another example of a DNA sequencing technique that can be used is ion semiconductor sequencing using, for example, a system sold under the trademark ION TORRENT by Ion Torrent by Life Technologies (South San Francisco, CA). Ion semiconductor sequencing is described, for example, in Rothberg, et al., An integrated semiconductor device enabling non-optical genome sequencing, Nature 475:348-352 (2011); U.S. Pub. 2010/0304982; U.S. Pub. 2010/0301398; U.S. Pub. 2010/0300895; U.S. Pub. 2010/0300559; and U.S. Pub. 2009/0026082, the contents of each of which are incorporated by reference in their entirety.

Another example of a sequencing technology that can be used is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured, and the identity of the first base is recorded. The 3′ terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Other suitable sequencing technologies may include single molecule, real-time (SMRT) technology of Pacific Biosciences (in SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW) where the fluorescent label is excited and produces a fluorescent signal, and the fluorescent tag is cleaved off. Detection of the corresponding fluorescence of the dye indicates which base was incorporated); nanopore sequencing (DNA is passed through a nanopore and each base is determined by changes in current across the pore, as described in Soni & Meller, 2007, Progress toward ultrafast DNA sequence using solid-state nanopores, ClinChem 53 (11): 1996-2001); chemical-sensitive field effect transistor (chemFET) array sequencing (e.g., as described in U.S. Pub. 2009/0026082); and electron microscope sequencing (as described, for example, by Moudrianakis, E. N. and Beer M., in Base sequence determination in nucleic acids with the electron microscope, III. Chemistry and microscopy of guanine-labeled DNA, PNAS 53:564-71 (1965)).

Sequencing according to embodiments disclosed herein generates a plurality of reads. Reads according to the disclosure generally include sequences of nucleotide data less than about 150 bases in length, or less than about 90 bases in length. In certain embodiments, reads are between about 80 and about 90 bases, e.g., about 85 bases in length. In some embodiments, methods disclosed herein are applied to very short reads, e.g., less than about 50 or about 30 bases in length. Sequence read data can include the sequence data as well as meta information.

Sequence read data can be stored in any suitable file format including, for example, VCF files, FASTA files or FASTQ files, as are known to those of skill in the art. FASTA is originally a computer program for searching sequence databases and the name FASTA has come to also refer to a standard file format. See Pearson & Lipman, 1988, Improved tools for biological sequence comparison, PNAS 85:2444-2448. A sequence in FASTA format begins with a single-line description, followed by lines of sequence data. The description line is distinguished from the sequence data by a greater-than (“>”) symbol in the first column. The word following the “>” symbol is the identifier of the sequence, and the rest of the line is the description (both are optional). There should be no space between the “>” and the first letter of the identifier. It is recommended that all lines of text be shorter than 80 characters. The sequence ends if another line starting with a “>” appears; this indicates the start of another sequence.

The FASTQ format is a text-based format for storing both a biological sequence (usually nucleotide sequence) and its corresponding quality scores. It is similar to the FASTA format but with quality scores following the sequence data. Both the sequence letter and quality score are encoded with a single ASCII character for brevity. The FASTQ format is a de facto standard for storing the output of high throughput sequencing instruments such as the Illumina Genome Analyzer. Cock et al., 2009, The Sanger FASTQ file format for sequences with quality scores, and the Solexa/Illumina FASTQ variants, Nucleic Acids Res 38 (6): 1767-1771.

Certain embodiments disclosed herein provide for the assembly of sequence reads. In assembly by alignment, for example, the reads are aligned to each other or to a reference. By aligning each read, in turn to a reference genome, all of the reads are positioned in relationship to each other to create the assembly. In addition, comparing 113 the sequence read to a reference sequence by, e.g., aligning or mapping, can also be used to identify variant sequences within the sequence read. In some embodiments, reads are aligned (e.g., to a reference) using Burrows-Wheeler Transform (BWT), which includes indexing the reference. A read is aligned to the reference using, for example, Burrow-Wheeler Aligner (BWA), e.g., using bwa-short to align each read to a reference, and the output can be a Binary Alignment Map (BAM) including, for example, a CIGAR string.

Computer programs for assembling reads are known in the art. Assembly can be implemented, for example, by the program “The Short Sequence Assembly by k-mer search and 3′ read Extension” (SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501). SSAKE cycles through a table of reads and searches a prefix tree for the longest possible overlap between any two sequences. SSAKE clusters reads into contigs. Other read assembly programs include: Forge Genome Assembler (see, e.g., DiGuistini et al., 2009, De novo sequence assembly of a filamentous fungus using Sanger, 454 and Illumina sequence data, Genome Biology, 10: R94); ABySS (Simpson et al., 2009, ABySS: A parallel assembler for short read sequence data, Genome Res., 19 (6): 1117-23).

In some embodiments, read assembly uses Roche's GS De Novo Assembler, known as gsAssembler or Newbler (NEW assemBLER), which is designed to assemble reads from the Roche 454 sequencer (described, e.g., in Kumar & Blaxter, 2010, Comparing de novo assemblers for 454 transcriptome data, Genomics 11:571 and Margulies 2005). Newbler accepts 454 Flx Standard reads, and 454 Titanium reads as well as single and paired-end reads and optionally Sanger reads. Newbler can be accessed via a command-line or a Java-based GUI interface.

The sequence data, in the form of the sequence reads themselves, or the product of read assembly such as a contig or consensus sequence, may be analyzed by comparison 113 to a reference. Comparing sequence data to a reference may include any suitable method such as alignment. For example, individual sequence reads may be aligned, or “mapped”, to a reference, or sequence reads may be assembled and then aligned. Any suitable reference may be used including, for example, a published human genome (e.g., hg18 or hg36), sequence data from sequencing a related sample, such as a patient's non-tumor DNA, or some other reference material, such as “gold standard” sequences obtained by, e.g., Sanger sequencing of subject nucleic acid.

Comparing 113 sequence data to a reference allows for the identification of mutations or variants. Using techniques of the disclosure, substitutions, small indels, and larger alterations such as rearrangements, copy number variation, and microsatellite instability can be determined. In certain preferred embodiments, the sequence data is from tumor DNA and is compared 113 to a non-tumor reference (such as sequence data from the same patient's non-tumor DNA) to identify mutations and variants. The analysis can include the identification of a variety of chromosomal alterations (rearrangements, amplifications, or microsatellite instability) with detection 125 of a boundary of such an alteration as well optionally sequence mutations (single base substitutions and small indels).

The output of comparing sequence data to a reference may be any suitable output or any suitable format. The output can be provided in the format of a computer file. In certain embodiments, the output is a FASTA file, FASTQ file, or VCF file. Output may be processed to produce a text file, or an XML file containing sequence data. Other formats include Simple UnGapped Alignment Report (SUGAR), Verbose Useful Labeled Gapped Alignment Report (VULGAR), and Compact Idiosyncratic Gapped Alignment Report (CIGAR) (Ning, Z., et al., Genome Research 11 (10): 1725-9 (2001)). In some embodiments, a sequence alignment is produced-such as, for example, a sequence alignment map (SAM) or binary alignment map (BAM) file-comprising a CIGAR string (the SAM format is described, e.g., in Li, et al., The Sequence Alignment/Map format and SAMtools, Bioinformatics, 2009, 25 (16): 2078-9).

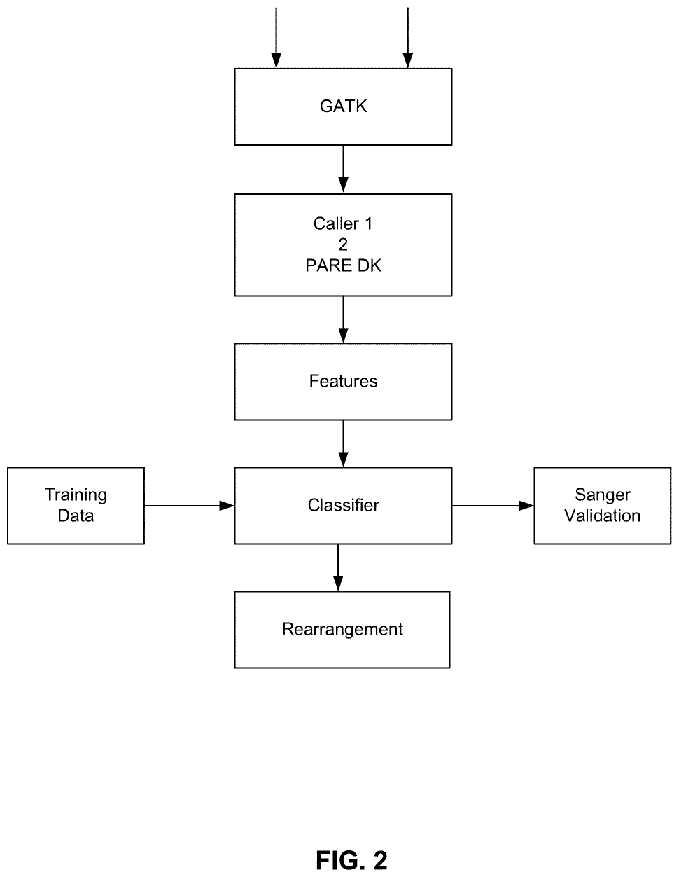

FIG. 2 diagrams the workflow for sequence analysis. Sequence data including either or both of tumor and matched normal sequence data from a patient are obtained as described above. That sequence data is compared 113 to a reference. Such a comparison may include alignment to the reference and may be implemented using a known tool such as the Genome Analysis Toolkit (GATK), BWA, or others. A tool, or caller, is then used to identify 125 at least one rearrangement or alteration within the sequence data. In fact, multiple alterations and mutations may be identified, and each type of mutation or alteration may be identified using a dedicated tool or caller to, e.g., implement any of the techniques described below.

Analysis includes using a caller tool to identify at least one alteration-specifically, to detect 125 at least one boundary of an alteration or rearrangement. The processes of identifying rearrangements, alterations, and mutations also gives a number of outputs, referred to here as features, that are used as inputs to the classification model. Features include all manner of output from the sequencing, analysis, and variant calling pipelines. For example, features may include the FASTQ quality score for any given base in the sequence data. In preferred embodiments, features include at least one instance of a confidence score, or probability score, that is output by the variant caller when a variant is identified.

At least one caller within the workflow is used in the detection 125 of a mutation or boundary of a structural alteration. In some embodiments, the output of that caller tool describes a rearrangement in the DNA from the sample, relative to the reference. For example, the caller may pass a genomic coordinate, stating that the coordinate represents the boundary of an inversion or translocation. Different tools may be used (any one or more of them) to identify one or a plurality of different rearrangement boundaries. Any suitable technique may be used to detect 125 the structural alteration.

For example, a boundary of a structural alteration may be detected 125 by a personalized analysis of rearranged ends (PARE). In some embodiments, detecting 125 the boundary includes sequencing a fragment of the DNA by paired-end sequencing to obtain a pair of paired-end reads, and mapping the pair of paired end reads to the reference. Here, when the pair of paired-end reads exhibit a discordant mapping to the reference, the fragment includes the boundary. For additional detail, see U.S. Pub. 2015/0344970, incorporated by reference.

Embodiments of PARE provide for the identification of patient-specific rearrangements in tumor samples. In preferred embodiments, PARE includes the analysis of mate-paired tags. Genomic DNA from a sample is purified, sheared and used to generate libraries with mate-paired tags (e.g., about 1.4 kb apart). Libraries may be digitally amplified by emulsion polymerase chain reaction (PCR) on magnetic beads and 25 bp mate-paired tags are sequenced (e.g., using sequencing-by-ligation of McKernan, 2009, Genome Res 19:1527-1541, incorporated by reference). About 200 million 25 base reads may be obtained for each sample where each read aligns perfectly and is uniquely localized in the reference human genome (e.g., hg18). Typically, tens of millions of mate-paired reads in which both tags map perfectly to the reference will be obtained. Mate-paired tags mapping the reference genome uniquely and without mismatches are analyzed for aberrant mate-pair spacing, orientation and ordering. Mate pairs mapping to the same chromosome at appropriate distances (about 1.4 kb) and in the appropriate orientation and ordering are considered concordant. Discordant mate pairs are candidates for finding alterations, such as rearrangements and/or copy number alterations. Various approaches may use PARE data to show alteration or rearrangement boundaries. One approach involves searching for tags whose mate-pairs map to different chromosomes, indicating inter-chromosomal rearrangements or translocations.

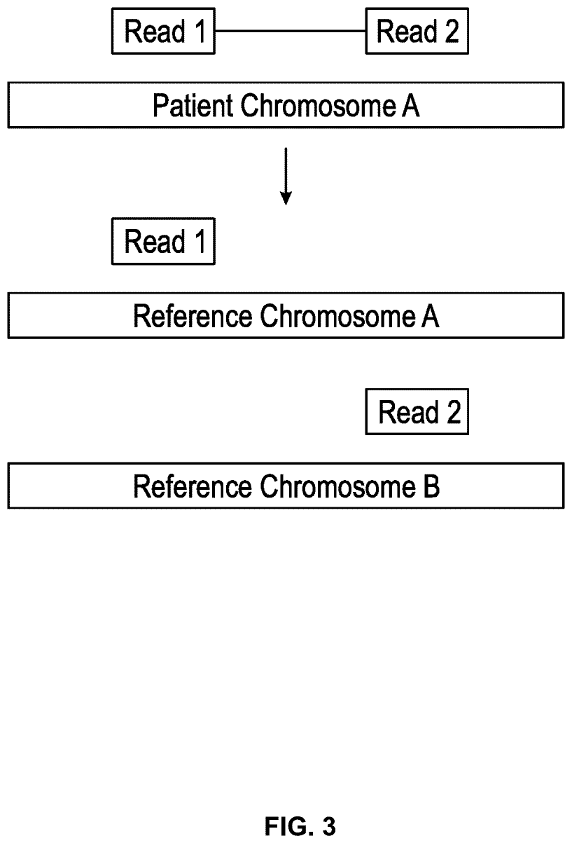

FIG. 3 illustrates detecting a translocation from mate-pair tags. Mate pair tags from one fragment of patient DNA, in which the tags map to different chromosomes of the reference. The high physical coverage of breakpoints provided by the about 40 million mate-paired sequences per sample suggest that a large fraction of such translocations can be identified. End sequences from such mate-paired tags maybe grouped into 1 kb bins and those bin pairs that are observed at least 5 times are analyzed further. The requirement for at least 5 occurrences minimized the chance that the presumptive fusion sequences represent incorrect mapping to the reference genome or artifacts of library construction. Finding multiple mate-pairs proximal to each other that both map to the different chromosomes can not only ensure that a translocation has been detected but can also allow one to precisely locate the boundary of the break and give a numerical genomic coordinate for the boundary (between 3′ and 5′ most-proximal mates). Another approach looks for mate pair tags to identify such pairs in which one member of the pair has a different copy number than the other.

For identification of candidate rearrangements associated with copy number alterations, a 10 kb boundary region of amplifications, homozygous deletions, or lower copy gains and losses is analyzed for neighboring discordant mate pair tags observed at least about 2 times in the tumor but not matched normal sample.

In certain embodiments, detecting 125 the boundary includes sequencing the DNA to determine a plurality of sequence tags, mapping the tags to the reference, and determining tag densities of mapped tags along portions of the reference in a process that may be referred to as digital karyotyping (DK). The DK techniques may be used within the PARE analyses discussed above, or may be used as described below on their own. In this technique, where a portion of the reference exhibits an anomalous tag density, an indel is detected in a corresponding portion of the DNA from the subject. An end (i.e., a terminus or boundary) of the indel corresponds to the boundary of the structural alteration. For additional detail, see U.S. Pat. No. 7,704,687, incorporated by reference.

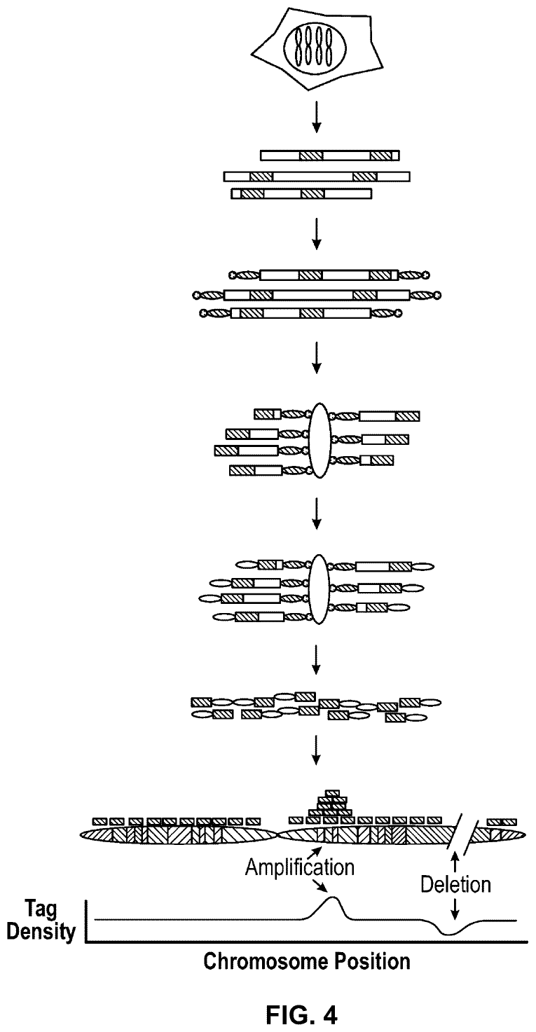

FIG. 4 illustrates output from a DK approach. For digital karyotyping, DNA is cleaved with a restriction endonuclease (mapping enzyme) that is predicted to cleave genomic DNA into several hundred thousand pieces, each on average <10 kb in size (Step 1). A variety of different endonucleases can be used for this purpose, and certain embodiments use SacI, with a 6-bp recognition sequence predicted to preferentially cleave near or within transcribed genes.

Biotinylated linkers are ligated to the DNA molecules (Step 2) and then digested with a second endonuclease (fragmenting enzyme) that recognizes 4-bp sequences (Step 3). As there are on average 16 fragmenting enzyme sites between every two mapping enzyme sites, the majority of DNA molecules in the template are expected to be cleaved by both enzymes and thereby be available for subsequent steps. DNA fragments containing biotinylated linkers are separated from the remaining fragments using streptavidin-coated magnetic beads (Step 3). New linkers, containing a 5-bp site recognized by Mmel, a type IIS restriction endonuclease, are ligated to the captured DNA (Step 4). The captured fragments are cleaved by Mmel, releasing 21 bp tags (Step 5). Each tag is thus derived from the sequence adjacent to the fragmenting enzyme site that is closest to the nearest mapping enzyme site. Isolated tags are self-ligated to form ditags, PCR amplified en masse, concatenated, cloned, and sequenced (Step 6). As described for SAGE (Velculescu, 1995, Science 270:484-487, incorporated by reference), formation of ditags provides a robust method to eliminate potential PCR induced bias during the procedure. Current automated sequencing technologies identify up to 30 tags per concatamer clone, allowing for analysis of 100,000 tags per day using a single 384 capillary sequencing apparatus. Finally, tags are computationally extracted from sequence data, matched to precise chromosomal locations, and tag densities are evaluated over moving windows to detect abnormalities in DNA sequence content (Step 7).

Whether using PARE, DK, PARE with DK, or some other technique, one or more of the caller tools in the analysis pipeline may detect 125 a boundary of an alteration or rearrangement. The method 101 includes comparing 113 the sequence reads to a reference to detect 125 at least one boundary of a structural alteration. That “called boundary” is passed to a classification model that classifies the called boundary as true or not, i.e., validates 129 the detected boundary as present in the DNA from the sample. Thus, the method 101 includes validating 129 the detected boundary as present in the DNA using a classification model trained on a training data set of sequences that include known structural alterations.

Additionally or alternatively, one or more of the caller tools in the analysis pipeline may detect 125 a mutation (such as a small indel or a substitution (e.g., SNV)). The method 101 includes comparing 113 the sequence reads to a reference to detect 125 the mutation. That “called mutation” is passed to a classification model that classifies the called mutation as true or not, i.e., validates 129 the detected mutation as present in the DNA from the sample. Thus, the method 101 includes validating 129 the detected mutation as present in the DNA using a classification model trained on a training data set of sequences that include known mutations.

Methods may include training the classification model by providing the training data set to the classification model and optimizing parameters of the classification model until the classification model produces output describing the known mutations or structural alterations.

Any suitable classification model may be used such as, for example, a neural network, a random forest, Bayesian classifier, logistic regression, support vector machine, principal component analysis, decision tree, gradient-boosted tree, multilayer perceptron, one-vs-rest, and Naive Bayes. It should be noted that those machine learning models (classification models) cannot practically be performed manually using paper and pen as the iterative processes, matrix operations, and computational complexity involved in these models make manual execution impractical for real-world datasets. Tasks such as gradient descent, eigenvalue calculations, and the handling of large data volumes require computational power far beyond what can be achieved manually, and attempting to do so would be infeasible and error-prone.

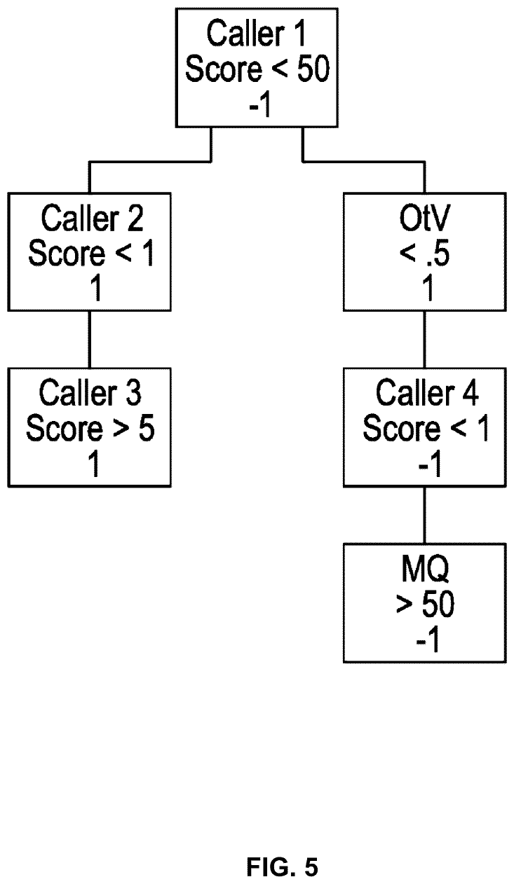

FIG. 5 illustrates a random forest classification model as may be implemented within a system disclosed herein. Random forests implement a plurality of decision trees, in which an input is entered at the top and as it traverses down the tree the data gets bucketed into smaller and smaller sets. The random forest combines, or forming an ensemble of, decision trees. Each tree contributes weekly to the classification, but as an ensemble, the forest is a strong classifier. A decision tree is a decision support tool that uses a tree-like graph or model of decisions and their possible consequences, including chance-event outcomes, resource costs, and utility. A decision tree embodies a number of yes/no questions that assess the probability that the detected mutation or boundary is true.

To train a random forest, for some number of trees T, a number N of cases are sampled at random with replacement to create a subset of the data. The subset may be, e.g., about 66% of the total set. At each node: (i) for some number m, m predictor variables are selected at random from all the predictor variables; (ii) the predictor variable that provides the best split, according to some objective function, is used to do a binary split on that node; and (iii) at the next node, choose another m variables at random from all predictor variables and do the same.

For a random forest, generally m<<number of predictor variables. When running a random forest, when a new input is entered into the system, it is run down all of the trees. The result may either be an average or weighted average of all of the terminal nodes that are reached. With a large number of predictors, the eligible predictor set will be quite different from node to node. As m goes down, both inter-tree correlation and the strength of individual trees go down.

So some optimal value of m is preferably discovered. Random forest runtimes are quite fast, and they are able to deal with unbalanced and missing data. The parameters that may be input into a random forest may include sample type; FASTQ quality score; alignment score; read coverage; and an estimated probability of error. The trained random performs forest classification and filtering of tumor specific alterations with confidence scoring. The trained model includes an ensemble of decision trees with different relative weights.

In some embodiments, the trained machine learning model leverages a sophisticated architecture, incorporating thousands of decision trees to assess unique combinations of feature values and assign a confidence score to each candidate variant. This computational improvement refines mutation detection by reducing reliance on rigid thresholds and rule-based methods, enabling the identification of low-frequency somatic mutations with greater sensitivity and specificity.

Random forests may be implemented in software such as de novo programs or a software package that performs operations described herein. One approach is implemented in the R package randomForest. Random forest classifiers are implemented by software packages such as ALGLIB, SQP, MatLab, and others. In certain preferred embodiments, the classification model is a random forest. In some embodiments, the classification model uses a neural network.

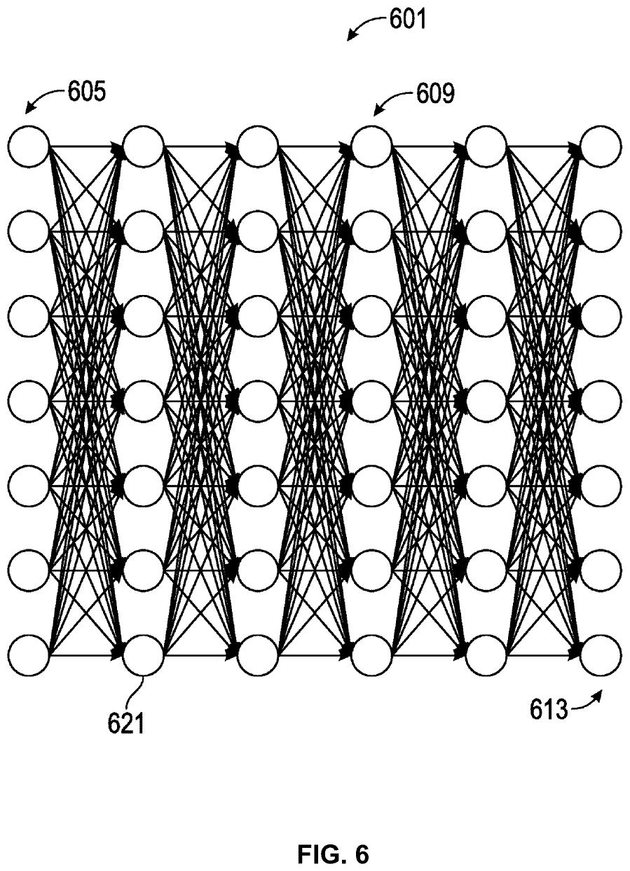

FIG. 6 illustrates a neural network 601 as may be implemented within a system disclosed herein. Neural networks are machine-learning tools that learn (progressively improve performance) to do tasks by considering examples, generally without task-specific programming. A neural network 601 typically includes a collection of connected units called artificial neurons or nodes 621. Each connection between nodes can transmit a signal to another node. The receiving node can process the signal(s) and then signal downstream nodes connected to it. Nodes may have state, generally represented by real numbers, typically between 0 and 1. Nodes and connections may also have a weight that varies as learning proceeds, which can increase or decrease the strength of the signal that it sends downstream. Further, they may have a threshold such that only if the aggregate signal is below (or above) that level is the downstream signal sent. Typically, nodes 621 are organized in layers 609. Different layers may perform different kinds of transformations on their inputs. Signals travel from the input layer 605, to the output layer 613, possibly after traversing the layers multiple times.

Embodiments disclosed herein implement a Naïve Bayes classification model. Naïve Bayes classifiers are a family of simple probabilistic classifiers based on applying Bayes' theorem with strong (naïve) independence assumptions between the features. The featured image is the equation—with P(A|B) is posterior probability, P(B|A) is likelihood, P (A) is class prior probability, and P(B) is predictor prior probability. In certain embodiments, logistic regression provides a classification model.

Logistic regression is a powerful statistical way of modeling a binomial outcome with one or more explanatory variables. It measures the relationship between the categorical dependent variable and one or more independent variables by estimating probabilities using a logistic function, which is the cumulative logistic distribution. Another suitable classification model uses support vector machines (SVM). SVM is binary classification algorithm. Given a set of points of 2 types in N dimensional space, SVM generates a (N−1) dimensional hyperplane to separate those points into 2 groups. Here, detected boundaries represent points in the space of input variables and an SVM may separate those points into true boundaries and experimental noise.

Principal Component Analysis (PCA) may be used as the classification model. PCA is a statistical procedure that uses an orthogonal transformation to convert a set of observations of possibly correlated variables into a set of values of linearly uncorrelated variables called principal components.

The role of the classification model includes validating 129 the genomic features that are identified by variant calling. In particular, the classification model validates 129 whether an identified boundary of a genomic alteration should be reported as “true”, or present in the subject DNA.

To this end, the classification model may be trained on a training data set. The training data set may include various subcomponents. In some embodiments, the training data set includes at least three sub-component data sets: real world clinical data (e.g., obtained by NGS), gold standard data (e.g., as may be obtained by Sanger sequencing of test samples), and simulated data sets that may be used to present known hard-to-detect mutations and alterations to the variant calling pipeline.

In certain embodiments, the gold standard data set includes hundreds of millions of bases of whole exome Sanger sequencing. The gold standard dataset preferably includes tens of thousands of genomic rearrangements. This data set may include reference standards of millions of bases. In preferred embodiments, the gold standard data set includes at least a substantial portion of a human genome or chromosome (e.g., at least 50%) obtained by Sanger sequencing. Sanger reference data is known to have greater accuracy than next-generation sequencing data, and thus can be used to confirm the legitimacy of variations. The NGS sequencing reads of a patient's tumor sample, a patient's normal sample, or both may be filtered against a Sanger reference prior to being compared to each other to identify tumor-specific mutations. In some embodiments, sections of the NGS sequence from nucleic acid determined to contain a tumor-specific alteration through comparison to NGS sequencing reads of a patient's normal sample may subsequently be filtered against a Sanger sequencing reference in order to validate the mutation. Methods and systems of comparing next generation sequence reads with a Sanger sequencing reference are described in U.S. Pub. 2016/0273049, incorporated by reference.

Real world clinical data may be included in the training data. Any suitable real-world clinical data may be included. In preferred embodiments, the real-world clinical data includes results from sequencing assays performed as described herein. For example, the real-world clinical data of the training data set may include sequences derived from NGS analysis of ctDNA such as the results of generating millions of short reads by sequencing prior test nucleic acid, mapping the reads to a reference, and calling mutations and alteration from the mapping results, in which the real-world clinical data further includes at least one known alteration in the prior test nucleic acid relative to a reference such as hg18 or hg37. Preferably, the real-world clinical data includes the known alterations and mutations as provided by the expert curation of tumor genomes. In a most preferred embodiment, the real-world clinical data includes data from thousands of clinical tumors and also includes alterations and mutation calls made by the expert curation of those tumors.

Simulations may form an important component of the training data set. Simulation data may include any kind of in silico data formatted like clinical and/or gold standard data. Simulated data may be generated de novo or may be generated by modifying another clinical data set to include simulated features. Simulated data may include one or any combination of simulated features such as simulated mutations or alterations, simulated copy number variants, simulated micro-satellite instability, simulations of tumor clonality, or simulated degrees of tumor mutation burden, to name but a few. In preferred embodiments, the simulated data includes more than one billion bases of clinical genomes with simulated mutant spike-ins (i.e., added into the data in an in-silico fashion), and preferably includes at least one alteration, relative to a reference, such as an indel, an inversion, a translocation, a duplication, aneuploidy, or a copy number variant.

The training data is used to train the classification model and also to validate that the trained classification model will perform reliably when presented with data not yet presented to the classification model. Thus, for example, a neural network may be provided with the real-world clinical data (the sequences and the known variants). The neural network assigns weights across neurons, observes differences between the network outputs and the known results (e.g., the variants called by expert curation over thousands of tumor samples). The neural network adjusts those weights until the network output converge on the known results. The simulated data may then be used to present the neural network with known, or suspected to be, difficult-to-detect mutation types such as clusters of small indels, or single nucleotide variants only a few bases away from indels. Once the neural network is trained, the ability of the neural network to call variants may be validated using the gold standard data. In fact, it should be appreciated that the gold standard data may be used to validate the entire variant calling pipeline or any step therein. In some embodiments, before the neural network is deployed clinically, the gold standard data is provided. Any deviation from the correct result is addressed. For examples, miss-calls by the neural network are back-propagated through the network to improve its calling. Whatever classification model is used, the classification model is used for validating a detected mutation or boundary as present in the nucleic acid.

Thus, embodiments disclosed herein employ machine learning whereby a training set with ground truth is provided to the machine-learning algorithm to create an adaptively boosted classifier. The classifier is applied to a target set to create a high-confidence rearrangement call set.

Analytical pipelines disclosed herein generate high quality data to maximize the accuracy of sequencing results. Sequencing results include quality scores. Minimum values or ideal ranges for high quality data may be implemented as a pass-through criterion within the pipeline. Each metric may be marked as pass or fail. For NGS, base calls are given a probability score on how likely the base called is truly the base of the DNA. Quality scores indicate the probability of an incorrect base call and incorporated into the .bcl and .fastq formats. Quality scores are generated by a quality table that uses a set of quality predictor values. The quality table may be updated when characteristics of the sequencing platform change.

The classification model may be developed in any suitable environment, such as Python and Bash scripting languages. The model may use hardware parallelism to achieve the best performance. The classification model has relatively low resource requirements when compared to running the callers. Even for large data sets, training is on the order of minutes or hours using about 10 GB of memory.

Methods disclosed herein are used to detect and report 135 alterations such as large indels, inversions, copy number variants, or translocations. Methods may also include detecting and reporting mutations such as small indels and substitutions. In preferred embodiments, methods include detecting and reporting a number of single-nucleotide variants (SNVs).

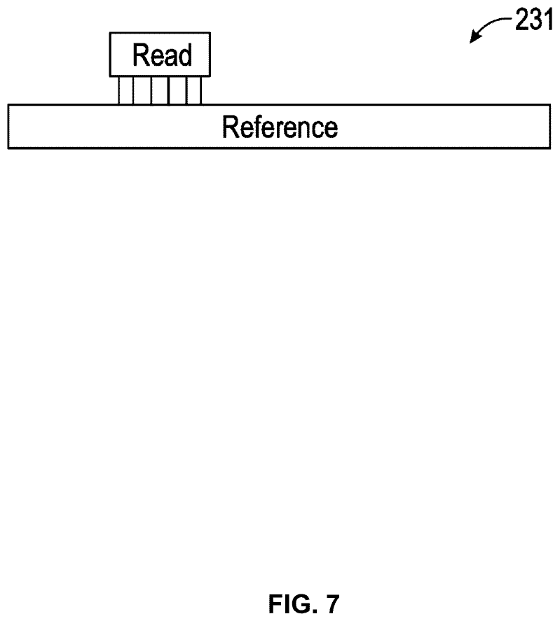

FIG. 7 illustrates calling an SNV. Calling an SNV generally involves comparing a sequence read to a reference and reporting any variation between them. Somatic variant callers score and filter data from alignment to call true sequence mutations. Alignment identifies candidate sequence mutations. Specifically, in some embodiments, the pipelines described herein score candidate sequence mutations and filter to resolve conflicts. After alignment to a reference genome, the next step is variant calling. A program (e.g., a de novo software application) examines the mapped data and reference genome side-by-side to determine the existence of sequence mutations (single base changes and small indels). In some embodiments, the program extracts candidate variants from alignment, then scores a number of (e.g., approximately 50) individual metrics for each variant and applies these scores both individually and in combination to identify bona fide sequence mutations and to exclude sequence artifacts. Any suitable program may be used to call somatic mutations.

MuTect is a somatic SNV caller that applies a Bayesian classifier to detect somatic mutations. It is sensitive in detecting low variant allele frequency (VAF) somatic variants. It also incorporates a series of filters to penalize candidate variants that have characteristics corresponding to sequencing artifacts to increase precision. SomaticSniper applies a Bayesian model to detect genotype change between the normal and tumor tissues, taking into account the prior probability of somatic mutation. Another Bayesian approach is JointSNVMix2, which jointly analyses paired tumor-normal digital allelic count data and has very high sensitivity in many different settings, but tends to be lower in precision. Variants may be reported in any suitable format such as the variant call format (* VCF; a standard tab-delimited format for storing variant calls).

It may be found that small changes (single base changes and indels) surrounded by wild-type sequence are relatively easy to align and call. Structural alterations as discussed above (large indels, Amplifications, Rearrangements) are highly variable between tumors and are not subject to predictable rules. As such, those structural alterations may be found to be difficult to align, detect, report, and describe.

Most somatic variant callers are not designed nor intended for analyzing tumor/normal pairs, and in such circumstances the trained classification model may allow for calling somatic variants between tumor/normal pairs that otherwise can't reliably be called with existing tools.

Additionally, variant calling for tissue differs for plasma because of the depth of sequencing coverage needed for plasma.

Due to the significant difficulties that may be presented in calling variants from cell-free DNA from plasma, the use of the classification model may be the step that provides for reliable and clinically useful variant calling and in particular for calling boundaries of alterations. The classification model is trained using training data as described above.

Preferably, the training data set includes a plurality of known single-nucleotide variants (SNVs). Methods disclosed herein may include detecting at least one SNV in the DNA; and validating the detected SNV as present in the DNA using the classification model, wherein the report describes the DNA as including the SNV. Thus, methods described herein accurately filter critical scoring conflicts commonly found in the NGS data and provide for clinically useful variant calling.

Methods disclosed herein may target patient sequences known to relate to a disease or condition. For example, if the target nucleic acid includes ctDNA, then screening may cover genes associated with cancer. Genes known to be associated with a variety of cancers include ABL1, AKT1, AKT2, ALK, APC, AR, ARID1A, ARID1B, ASXL1, ATM, ATRX, BAP1, BRAF, BRCA1, BRCA2, CBL, CCND1, CCNEI, CDH1, CDK4, CDK6, CDKN2A, CEBPA, CREBBP, CTNNB1, DAXX, DNMT3A, EGFR, ERBB2, ERBB3, ERBB4, EZH2, FBX27, FGFR2, FGFR3, FGFR4, FLT3, FOXL2, GATA1, GATA2, GNA11, GNAQ, GNAS, HNFIA, HRAS, IDH1, IDH2, IGFIR, IGF2R, IKZF1, JAK1, JAK2, JAK3, KDR, KITKRAS, MAMLI, MDM2, MDM4, MED12, MEN1, MET, MLH1, MLL, MPL, MSH2, MSH6, MYC, MYCN, MYD88, NF1, NF2, NOTCH1, NOTCH2, NOTCH3, NOTCH4, NPM1, NRAS, PALB2, PAXS, PBRM1, PDGFRA, PDGFRB, PIK3CA, PIK3R1, PMS2, PTCH1, PTEN, PTPN11, RB1, RET, RNF43, ROS1, RUNX1, SF3B1, SMAD2, SMAD3, SMAD4, SMARCBI, SMO, STAG2, STK11, TET2, TGFBR2, TNFAIP3, TP53, TSC1, TSC2, TSHR, VHL, and WT1a. Mutations in those genes may be used to diagnose, classify tumor subtypes, determine prognoses, monitor tumor progression, and establish appropriate therapies. Types of mutations identified using the systems and methods disclosed herein may include any type of mutation known in the art, including, for example, an insertion, a deletion, a copy number alteration, and/or a translocation.

The analytical pipeline described herein is preferably used to identify and validate the reporting of an alteration in sample nucleic acid relative to a reference. The pipeline workflow may start with, e.g., FASTQ files for both tumor and the matched normal sequencing reads, which may be processed using Genome Analysis Toolkit (GATK) to provide BAM files. Optionally, the calls along with the feature set is provided to the machine-learning model. After training, the model calculates the probability for each call, yielding a high-confidence somatic mutation call set.

There may be separate optimization of pipelines for mutation calling and alteration calling. In some embodiments, the mutation calling pipeline is trained using 300+ million bases of whole-exome Sanger sequences of matched tumor/normal pairs. The size and breadth of the Sanger dataset allows the pipeline to make accurate variant calls down to 2% MAF. It works well in difficult-to-analyze regions of the genome, such as GC-rich regions and highly repetitive regions. The alteration calling pipeline is preferably trained and tested on more than 10,000 suspected alterations such as indels, translocation, copy number variants, inversions, and fusions.

Whole genome translocation analysis of 200 matched T/N pairs is performed using a caller and a classification model trained on a large training data set. It is believed that the large training set allows the alteration-calling pipeline to have clinical utility to detect alterations in ctDNA by NGS. That is, it is understood that methods described herein are what allow for the detection of tumor-specific structural alterations by NGS from ctDNA. Those large structural variations (large indels, amplifications, rearrangements) are highly variable between tumors but nevertheless are important clinical targets in the management of cancer patients. Furthermore, those alterations are some of the most therapeutically targeted among FDA-approved indications (imatinib, crizotinib, trastuzumab, etc.). Thus, methods disclosed herein may have particular utility in determining an effective course of treatment for a patient via a minimally invasive procedure (e.g., an assay from blood or plasma).

In particular, use of the classification model adds accuracy and reliability for clinical utility to alteration calling as described here. Embodiments of the disclosure detect alterations within ctDNA through techniques such as Paired Analysis Rearranged Ends (PARE) and Digital Karyotyping (DK). PARE Identifies rearrangements. Fusions reads may look like “noise” to the aligners and may otherwise be filtered out. Instead, here, methods may include obtaining one or more mate-pairs of sequencing data from nucleic acid; aligning the mate-pairs to a reference (e.g., a human genome); identifying discordant mate-pair reads as possible rearrangements; and having a classification model validate the presence of the rearrangement.

To identify amplifications and deletions, DK may be used. For DK, specific barcodes are incorporated during library prep to index the tags that result from sequencing; the sample is sequenced, and the tags are aligned to the reference. Tag densities are observed across the genome to identify outliers. Such techniques may be used to identify amplifications and deletions. Whatever methods are used for detecting alterations, those methods are preferably validated against whole-exome PCR and Sanger sequencing from cancer DNA. When the methods are applied to a sample, the alteration(s) provisionally detected by the caller are validated for reporting by the trained classification model.

Methods disclosed herein have a number of useful and beneficial applications. Methods may be used for the therapeutic interpretation of cancer panels. Methods may be used to uniquely detect and prioritize variants and patients for treatments. Methods of the disclosure may be fully automated and scalable with a rapid turnaround time (e.g., less than a day). Clinical and research reports may be provided 135 describing, e.g., tumor genetics including, for example, clonality. Reports may identify tumor specific mutations including driver and passenger mutations. In preferred embodiments, a report is provided 135 that describes the DNA from the subject as including the structural alteration.

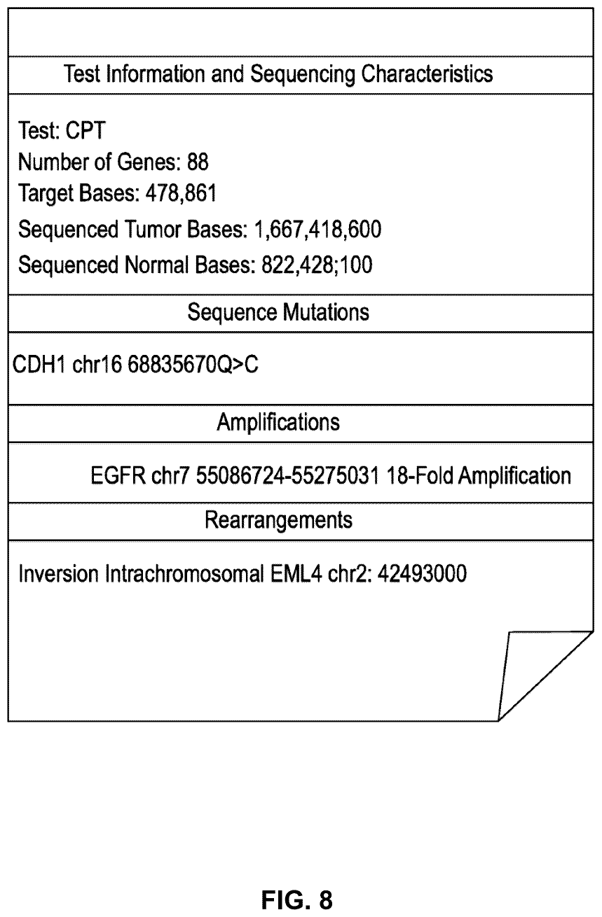

FIG. 8 shows a report according to some embodiments. Reports may allow methods disclosed herein to be useful in the continuing care of a cancer patient. After beginning a treatment regimen, the patient's tumor sequence can be analyzed again using the same methods. This second analysis can determine whether there are more or fewer mutations, which is indicative of whether the cancer is progressing. Embodiments disclosed herein may be implemented as a system to perform methods described herein.

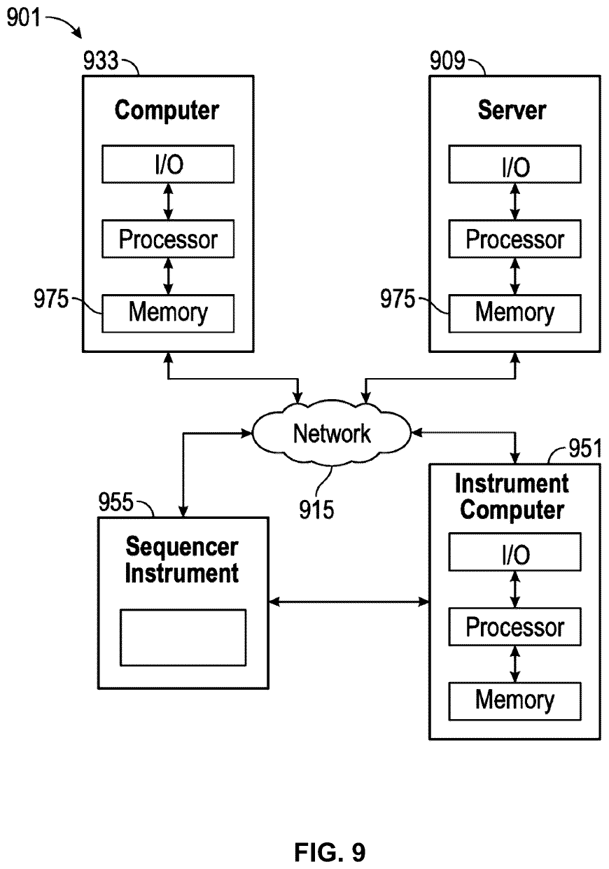

FIG. 9 shows a system 901 according to certain embodiments. The system 901 includes a computer 933 that has a processor coupled to memory and one or more input/output devices. The system 901 may also include either or both of a server computer 909 and a sequencing instrument 955, optionally with a sequencer computer 951. Each computer includes at least one processor coupled to memory and one or more input/output devices. Memory as used herein may be taken to refer to tangible, non-transitory computer readable storage media and may include one or any combination of RAM, ROM, hard drives, solid-state drives, optical disks, and removable drives. Those memory devices, taken together, may be collectively referred to as a memory subsystem 975 of the system 901. The memory subsystem 975 preferably has instructions stored therein that, when executed by the at least one processor, cause the system 901 to obtain sequence reads from DNA from a sample from a subject; comparing the sequence reads to a reference to detect at least one boundary of a structural alteration; validating the detected boundary as present in the DNA using a classification model trained on a training data set of sequences that include known structural alterations; and providing a report that describes the DNA from the subject as including the structural alteration.

### Exemplary Computing Environments

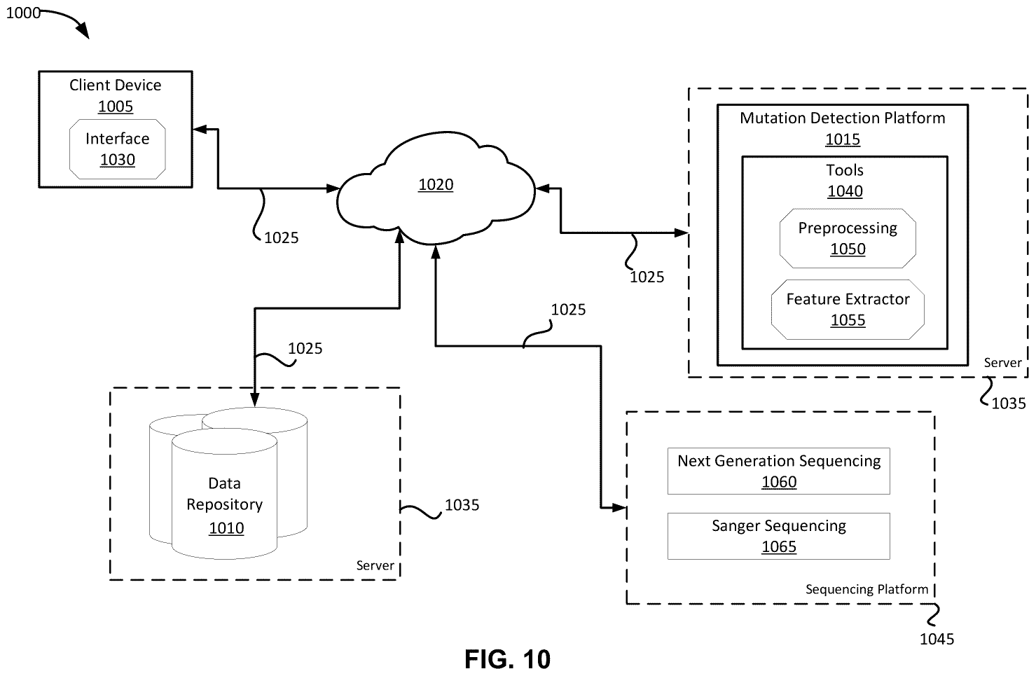

FIG. 10 shows a computing environment 1000 for implementing NGS workflows in accordance with various embodiments. The computing environment 1000 includes a client device 1005, a server 1035, a sequencing platform 1045, and a network 1020 connecting to components of the computing environment 1000. Although FIG. 10 illustrates a particular arrangement of the client device 1005, the server 1035, the sequencing platform 1045, and the network 1020, this disclosure contemplates any suitable arrangement of these components and additional components. As an example, and not by way of limitation, two or more client devices 1005, the server 1035, and the sequencing platform 1045 may be connected to each other directly, bypassing the network 1020. As another example, two or more client devices 1005, the server 1035, and the sequencing platform 1045 may be physically or logically co-located with each other in whole or in part. Moreover, although FIG. 10 illustrates a particular number of the components, this disclosure contemplates any suitable number of components (e.g., client devices 1005, servers 1035, sequencing platforms 1045, and networks 1020). As an example, and not by way of limitation, computing environment 1000 may include multiple client devices 1005, multiple servers 1035, multiple sequencing platforms 1045, and multiple networks 1020.

This disclosure contemplates any type of network 1020 familiar to those skilled in the art that may support data communications using any of a variety of available protocols including without limitation TCP/IP (transmission control protocol/Internet protocol), SNA (systems network architecture), IPX (Internet packet exchange), AppleTalk®, and the like. Merely by way of example, network(s) 1020 may be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Links 1025 may connect a client device 1005, a server 1035 or a unit thereof (e.g., a data repository 1010, a mutation detection platform 1015), or a sequencing platform 1045 or a unit thereof (e.g., a NGS unit 1060, or a Sanger sequencing unit 1065) to a network 1020 or to each other. This disclosure contemplates any suitable links 1025. In particular embodiments, one or more links 1025 include one or more wireline (such as for example Digital Subscriber Line (DSL) or Data Over Cable Service Interface Specification (DOCSIS)), wireless (such as for example Wi-Fi or Worldwide Interoperability for Microwave Access (WiMAX)), or optical (such as for example Synchronous Optical Network (SONET) or Synchronous Digital Hierarchy (SDH)) links. In particular embodiments, one or more links 1025 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 1025, or a combination of two or more such links 1025. Links 1025 need not necessarily be the same throughout the computing environment 1000. One or more first links 1025 may differ in one or more respects from one or more second links 1025.

A client device 1005 is an electronic device including hardware, software, or embedded logic components or a combination of two or more such components and capable of interacting with the server 1035 or a unit thereof (e.g., the data repository 1010, the mutation detection platform 1015) and the sequencing platform 1045 or a unit thereof (e.g., the NGS unit 1060, the Sanger sequencing unit 1065), optionally via the network 1020. The client device 1005 may include various types of computing systems such as portable handheld devices such as cell phones, general purpose computers such as personal computers and laptops, workstation computers, wearable devices, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone), tablets (e.g., iPad®), personal digital assistants (PDAs), and the like. Wearable devices may include Google Glass® head mounted display, and other devices. The client device 1005 may be capable of executing various different applications such as various Internet-related apps, communication applications (e.g., E-mail applications, short message service (SMS) applications) and may use various communication protocols. This disclosure contemplates any suitable client device 1005 configured to generate and output product target discovery content to a user. For example, users may use client device 1005 to execute one or more applications, which may generate one or more discovery or storage requests that may then be serviced in accordance with the teachings of this disclosure. The client device 1005 may provide an interface 1030 (e.g., a graphical user interface) that enables a user of the client device 1005 to interact with the client device 1005. The client device 1005 may also output information to the user via this interface 1030 (e.g., displaying a report). Although FIG. 10 depicts only one client device 1005, any number of client devices 1005 may be supported.

The client device 1005 is capable of inputting data, generating data, and receiving data. For example, a user of the client device 1005 may send out a request to perform a genetic screening or mutation detection using the interface 1030. The request may be sent out through the network 1020 to the sequencing platform 1045, and NGS or targeted NGS (e.g., WES) may be performed on a sample based on the request using the NGS unit 1060. After the sequencing, the NGS sequence reads or NGS sequencing data may be automatically sent to the server 1035 through the network 1020 for further processing. For example, the NGS data may be sent to the mutation detection platform 1015 to determine candidate mutations and extract corresponding feature information using tools 1040 (e.g., through the preprocessing unit 1050 and the feature extractor 1055). Reference sequencing data may be extracted or retrieved from the data repository 1010 and sent to the mutation detection platform 1015 together with the NGS data. Additional information such as demographic information or configuration files may also be extracted or retrieved from the data repository 1010 using the tools 1040. The extracted information together with the reference data may be further processed using the mutation detection platform 1015 to select somatic mutations. The somatic mutation information may be sent back to the sequencing platform 1045 to perform confirmatory sequencing using the Sanger sequencing unit 1065. The somatic mutation information may also be communicated to the user of the client device 1005 and the user may decide whether to perform confirmation testing or determine personalized treatments. The Sanger sequencing data may be sent back to the server 1035 or the mutation detection platform 1015 for subsequent analysis. For example, the NGS data and the Sanger sequencing data may be used together to determine if the sample comprises the selected somatic mutations, or if a subject where the sample is obtained has developed a genetic condition (e.g., a disorder, a disease, or a cancer). The somatic mutation information or the disease diagnosis information may be transmitted to the client device 1005 via the network 1020. The data (e.g., the NGS data, the Sanger sequencing data, the feature information, the demographic information, and/or somatic mutation information) may also be sent and stored in the data repository 1010.

A data repository 1010 is a data storage entity (or sometimes entities) into which data has been specifically partitioned for an analytical or reporting purpose. The data repository 1010 may be used to store data and other information generated or used by the mutation detection platform 1015, the client device 1005, and/or the sequencing platform 1045. For example, one or more of the data repositories 1010 may be used to store data and information to be used as input into the mutation detection platform 1015 for generating a final variant call report (e.g., a somatic mutation report as shown in FIG. 8). In some instances, the data and information relate to genetic sequences (genomic, exomic, and/or targeted), high-confidence variants, information on variant type and clinical significance, and other information used by the mutation detection platform 1015 when performing assaying functions. The data repositories 1010 may reside in a variety of locations including the server 1035. For example, a data repository used by a server 1035 may be local to the server 1035 or may be remote from the server 1035 and in communication with the server 1035 via a network-based or dedicated connection of network 1020. Data repositories 1010 may be of different types or of the same type. In certain examples, a data repository 1010 may be a database which is an organized collection of data stored and accessed electronically from one or more storage devices such as one or more servers 1035. The one or more servers 1035 may be configured to execute a database application that provides database services to other computer programs or to computing devices (e.g., client device 1005 and mutation detection platform 1015) within the computing environment 1000, as defined by a client-server model. One or more of these databases may be adapted to enable storage, update, and retrieval of data to and from the database in response to SQL-formatted commands or like programming language that is used to manage databases and perform various operations on the data within them.

The mutation detection platform 1015 comprises a set of tools 1040 for the purpose of analyzing and visualizing data (e.g., data stored in the data repository 1010, data generated by the sequencing platform 1045, or the data sent from the client device 1005). The mutation detection platform 1015 is used to execute a process to provide high-confidence somatic mutations based on NGS data. In the exemplary configuration depicted in FIG. 10, the set of tools 1040 include two units: a preprocessing unit 1050 and a feature extractor 1055. The preprocessing unit 1050 is capable of loading, processing, and saving data (e.g., accessed from the data repository 1010) to be used by the preprocessing unit 1050 itself and the feature extractor 1055. The feature extractor 1055 uses the processed data to identify high-confidence somatic mutations based on the NGS data. In some instances, the mutation detection platform 1015 is used together with the sequencing platform 1045 to: (i) generate NGS data for a patient sample (for whole genome or for regions of interest), (ii) obtain demographic information, configuration files, and reference data (e.g., from the data repository 1010), (iii) determine variant calls and associated feature information for the patient sample based on the NGS data, (iv) select somatic mutations based on the variant calls and associated feature information using a trained machine learning model, and (v) output information and/or reports. The candidate variants or somatic mutations can be further confirmed using the Sanger sequencing unit 1065 to confirm the variant calls or the selected somatic mutations for the sample with improved accuracy and specificity. The mutation detection platform 1015 may reside in a variety of locations including the server 1035. For example, a mutation detection platform 1015 used by a server 1035 may be local to the server 1035 or may be remote from the server 1035 and in communication with the server 1035 via a network-based or dedicated connection of network 1020. The mutation detection platform 1015 may be of different configurations or of the same configuration.

The server 1035 may be adapted to run one or more services or software applications that enable one or more embodiments described in this disclosure. In certain instances, server 1035 may also provide other services or software applications that may include non-virtual and virtual environments. In some examples, these services may be offered as web-based or cloud services, such as under a Software as a Service (SaaS) model to the users of client device 1005. Users operating client device 1005 may in turn utilize one or more client applications to interact with server 1035 to utilize the services provided by these components (e.g., database and rescue applications). In the configuration depicted in FIG. 10, server 1035 may include one or more components that implement the functions performed by server 1035. These components may include software components that may be executed by one or more processors, hardware components, or combinations thereof. It should be appreciated that various different device configurations are possible, which may be different from computing environment 1000. The example shown in FIG. 10 is thus one example of a computing environment and is not intended to be limiting.

Server 1035 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. Server 1035 may include one or more virtual machines running virtual operating systems, or other computing architectures involving virtualization such as one or more flexible pools of logical storage devices that may be virtualized to maintain virtual storage devices for the server. In various instances, server 1035 may be adapted to run one or more services or software applications that provide the functionality described in the foregoing disclosure. In some embodiments, the server 1035 is a physical or virtual machine for hosting applications, storing data, managing databases, or facilitating communication between systems that provides computing resources and services to other devices (clients) on a network. In some embodiments, the server 1035 is an on-premises server or a cloud-based server.

The computing systems in server 1035 may run one or more operating systems including any of those discussed above, as well as any commercially available server operating system. Server 1035 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, Sybase®, IBM® (International Business Machines), and the like.

In some implementations, server 1035 may include one or more applications to analyze and consolidate data feeds and/or data updates received from users of client devices 1005. As an example, data feeds and/or data updates may include, but are not limited to, in vivo feeds, in silico feeds, or real-time updates received from public studies, user studies, one or more third party information sources, and data streams (continuous, batch, or periodic), which may include real-time events related to sensor data applications, biological system monitoring, and the like. Server 1035 may also include one or more applications to display the data feeds, data updates, and/or real-time events via one or more display devices of client devices 1005.

The sequencing platform 1045 is configured to perform sequencing tasks including NGS and Sanger sequencing. The sequencing platform 1045 may be performed fully automatically with loaded samples, or performed semi-automatically with the help of a practitioner. As illustrated in FIG. 10, the sequencing platform 1045 may include two units: an NGS unit 1060 performing next-generation sequencing, and a Sanger sequencing unit 1065. In some instance, the sequencing platform 1045 may include additional units, such as a third-generation sequencing (TGS) unit (e.g., performing single molecule real-time (SMRT) sequencing and/or nanopore sequencing), a pyrosequencing unit, an Ion Torrent sequencing unit, and/or a sequencing by ligation (SOLID) unit. In some instances, the NGS unit 1060 is capable of performing the functions in the additional units.

NGS is a powerful technology that allows for the rapid sequencing of entire genomes or targeted regions of DNA or RNA. In some instances, the NGS unit 1060 performs a nucleic acid extraction process to isolate high-quality DNA or RNA from a biological sample. This may be followed by fragmentation, where the extracted nucleic acids are broken into smaller, more manageable pieces. This can be achieved through mechanical shearing, enzymatic digestion, or sonication.

The fragmented DNA or RNA is then prepared for sequencing as part of a library preparation process. This process includes the ligation of sequencing adapters, which adapters are short, double-stranded DNA sequences (or single-stranded RNA sequences) that are ligated to the ends of the fragments, allowing them to bind to the sequencing flow cell and facilitate amplification. The library preparation process may also involve additional steps10 to ensure that the fragments are of the appropriate size and concentration for sequencing. This can include size selection, where fragments of a specific length are isolated using gel electrophoresis or magnetic beads. The prepared library is then quantified and quality-checked using techniques such as quantitative PCR (qPCR) or bioanalyzer assays to ensure that it meets the requirements for sequencing. In some instances, the wet-lab procedures are performed by a trained practitioner.

Once the library is ready, it can be loaded onto the sequencing platform 1045 or the NGS unit 1060. Different NGS platforms may have their own sequencing chemistries and technologies, generally involving the attachment of the library fragments to a solid surface, amplification to create clusters or colonies of identical sequences, and sequencing-by-synthesis or other methods to read the nucleotide sequence of each fragment. The sequencing process performed by the NGS unit 1060 generates massive amounts of data (e.g., millions to billions of sequence reads or raw data), which can be then transferred to the server 1035 or the mutation detection platform 1015 for analysis. In some instances, the NGS unit 1060 or another component of the sequencing platform 1045 may analyze, process, or manage the sequencing data. For example, bioinformatics tools and algorithms may be employed to process raw sequencing data, which includes base calling, quality control, read alignment, and variant calling. High-performance computing systems and cloud-based platforms are often used to handle the computationally intensive tasks of sequence alignment and data analysis. Additionally, specialized software pipelines are used to assemble the sequenced reads into complete genomes or to identify genetic variants. The integration of artificial intelligence and machine learning algorithms may be further adopted to enhance the accuracy and efficiency of data analysis, enabling the identification of novel genetic markers and potential therapeutic targets.

The Sanger sequencing unit 1065 is configured to perform Sanger sequencing for determining the nucleotide sequence of DNA or RNA. The Sanger sequencing unit 1065 is capable of synthesis of a complementary DNA strand using a single-stranded DNA template (or an RNA template), a DNA polymerase enzyme, and a mixture of normal deoxynucleotides (dNTPs) and chain-terminating dideoxynucleotides (ddNTPs). The ddNTPs are fluorescently or radioactively labeled and lack a 3′ hydroxyl group, which prevents further elongation of the DNA strand upon incorporation. By including a small proportion of ddNTPs in the reaction, a series of DNA fragments of varying lengths is generated, each terminating at a specific nucleotide. The resulting DNA fragments are then separated by size using capillary electrophoresis or polyacrylamide gel electrophoresis. In capillary electrophoresis, an electric field is applied to a capillary tube filled with a polymer matrix, which allows the fragments to migrate based on their size. Smaller fragments move faster through the capillary, while larger fragments move more slowly. As the fragments pass through a detector, the fluorescent or radioactive labels are detected, and the sequence of the DNA or RNA is determined by analyzing the order of the labeled fragments. The sequence data can also be sent to the server 1035 or the mutation detection platform 1015 for analysis. In some instances, the Sanger sequencing data can be compiled and interpreted using the sequencing platform 1045 to reconstruct the original DNA or RNA sequence, validate of sequences obtained from the NGS unit 1060, or detect variants in the biological materials. Sanger sequencing remains a gold standard for its accuracy and reliability, particularly for smaller-scale sequencing tasks, diagnostic applications, and confirming genetic variations identified by other methods (e.g., the NGS method).

### III. Embodiments Related to Whole Genome Sequencing

### A. Exemplary Flowcharts

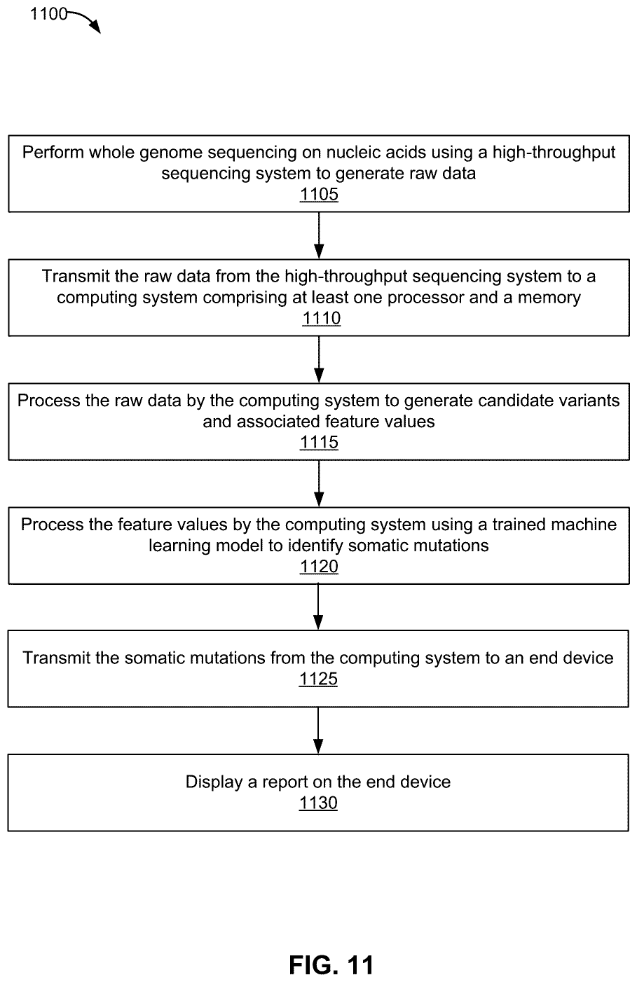

FIG. 11 is a flowchart illustrating a process 1100 for a genetic screening assay to identify and display high-confidence somatic mutations in accordance with embodiments. The processing depicted in FIG. 11 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 11 and described below is intended to be illustrative and non-limiting. Although FIG. 11 depicts the various processing steps occurring in a particular sequence or order, this is not intended to be limiting. In certain alternative embodiments, the steps may be performed in some different orders, or some steps may also be performed in parallel.

At block 1105, whole genome sequencing is performed on nucleic acids using a high-throughput sequencing system (e.g., a high-throughput sequencer) to generate raw data for further analysis. The high-throughput sequencing system can be deployed in the sequencing platform 1045, and the high-throughput sequencer may be the NGS unit 1060 described with respect to FIG. 10. The nucleic acids are extracted from biological samples obtained from a subject. These biological samples can include a wide range of sources, such as white blood cells or circulating cell-free DNA (ccfDNA) isolated from blood, epithelial cells collected from saliva, or tissue biopsies, which may include both fresh samples or formalin-fixed, paraffin-embedded (FFPE) samples. Additional sources of nucleic acids include buccal swabs, which collect DNA from cells lining the inside of the cheek, hair follicles, and urine, which may contain shed epithelial cells. Other bodily fluids, such as cerebrospinal fluid or amniotic fluid, are also valuable sources of nucleic acids, particularly for specific diagnostic or research purposes. In some embodiments, the nucleic acids are cell-free DNA (cfDNA) isolated from a liquid sample of the subject. In some embodiments, the cfDNA is about 5 ng to 10 ng.

The subject from whom the biological sample is obtained may be a patient with a known cancer diagnosis, an individual identified as having a predisposition to cancer based on genetic or clinical assessments, or a pregnant woman undergoing prenatal testing. In the case of cancer patients or individuals with a predisposition to cancer, whole genome sequencing provides critical insights into the genetic mutations, structural variations, or other genomic alterations associated with the disease. These insights enable personalized treatment strategies, risk assessment, and early intervention by identifying targeted therapies, monitoring disease progression, detecting minimal residual disease, and guiding clinical decision-making tailored to the patient's unique genomic profile. For pregnant women, whole genome sequencing is often performed on cell-free DNA (cfDNA) extracted from maternal blood to evaluate fetal genetic material non-invasively. This approach allows for the detection of chromosomal abnormalities, such as aneuploidies or structural variations, and provides valuable information for assessing the health and development of the fetus. The ability to sequence genomes in these contexts underscores the transformative role of genomics in advancing precision medicine, improving outcomes for both patients and unborn children, and enabling proactive healthcare decisions.

Techniques disclosed herein may include preparing nucleic acids for whole genome sequencing. First, the nucleic acids (e.g., genomic DNA) can be fragmented into smaller DNA fragments, typically ranging from 200 to 500 base pairs in length. This fragmentation process is achieved using mechanical methods, such as sonication or nebulization, or enzymatic methods, such as treatment with restriction enzymes. Fragmentation ensures that the entire genome is represented as manageable pieces that can be sequenced in parallel using high-throughput sequencing platforms. Following fragmentation, paired-end adapters are ligated to both ends of each DNA fragment. These adapters are short, synthetic oligonucleotides that can serve multiple purposes: they provide known sequences for primer binding during sequencing, enable the identification of fragment orientation, and optionally, allow for sample multiplexing when multiple libraries are sequenced in the same run. In some embodiments, unique molecular identifiers (UMIs) or barcodes may be used as part of the adapters. The ligation of paired-end adapters ensures that sequencing can occur from both ends of each DNA fragment (e.g., paired-end sequencing). Paired-end sequencing can further improve the accuracy of read alignment to the reference genome and facilitates the detection of structural variations, insertions, deletions, and repetitive sequences.

After adapter ligation, the ligated DNA fragments may be amplified through polymerase chain reaction (PCR) to generate a DNA library. This amplification step increases the quantity of DNA, ensuring that there is sufficient material for sequencing. Careful optimization of PCR conditions is critical to avoid introducing amplification bias or errors, which could affect downstream analysis. The resulting DNA library represents a comprehensive and randomized sampling of the original genome, with each fragment flanked by adapter sequences that are compatible with the sequencing platform. In some embodiments, PCR is not performed in the genetic screening assay.

Whole genome sequencing can be then performed on the prepared DNA library using paired-end sequencing or single-end sequencing technology. In paired-end sequencing, both ends of each DNA fragment are sequenced, producing two complementary reads for each fragment. This approach enhances the accuracy of read alignment and variant calling, as the paired reads provide positional and orientation information about the fragments in the context of the reference genome. Paired-end sequencing is particularly advantageous for detecting structural variants, resolving repetitive regions, and improving genome assembly. Single-end sequencing, on the other hand, involves sequencing only one end of each DNA fragment, generating a single read per fragment. Single-end sequencing is often faster, more cost-effective, and requires less computational power for data analysis. Single-end sequencing can be used for tasks such as gene expression profiling, certain types of transcriptomic analyses, and studies related to read depth. The choice between the two sequencing approaches may depend on the specific research or clinical goals, as well as considerations of cost, time, and computational resources. Raw data is then generated by the whole genome sequencing.

The raw data refers to the unprocessed output generated by the high-throughput sequencer. The raw data consists of large quantities of nucleotide sequences, which are represented as a series of base calls (A, T, C, and G for DNA or A, U, C, and G for RNA) and accompanied by quality scores that indicate the confidence of each base call. For example, in whole genome sequencing, the raw data includes millions or billions of short DNA fragments (sequence reads) that need to be aligned and assembled to reconstruct the genome. The raw data is stored in a specific format in a storage medium associated with the high-throughput sequencing system or in a local storage device communicatively coupled to the high-throughput sequencing system (e.g., the data repository 1010 described with respect to FIG. 10). Examples of raw data formats generated during sequencing processes include a variety of file types, each serving a specific purpose in storing and processing sequencing information. For instance, a .bcl file (Base Call file) contains raw base call and quality score data outputted directly from Illumina sequencers, while a .cbcl file (Compressed Base Call file) is a more compact version of the same data. A FASTQ file is one of the most widely used formats, storing both nucleotide sequences and their corresponding quality scores for downstream analysis. A. fast5 file is specific to Oxford Nanopore sequencing platforms and stores raw signal data, metadata, and base call information. Other examples include a .bax.h5 file, used by PacBio sequencing systems to store information about subreads, polymerase reads, and quality scores, a .subreads.bam file, which contains processed subread data in a compressed, indexed format, a FASTA file that stores only nucleotide sequences (without quality scores), a .dat file can serve as a raw signal data storage format, a .bam file (Binary Alignment/Map file) used to store aligned sequencing reads, and a .pod5 file for storing signal-level data and metadata in a more efficient and scalable manner. By leveraging different file formats, sequencing workflows can be tailored to accommodate the unique requirements of various platforms, analytical goals, and computational resources, thereby enhancing the accuracy, efficiency, and scalability of genomic research and clinical applications.

In some embodiments, the raw data generated during the WGS is raw signal data, which consists of unprocessed output directly obtained from the sequencing platform (e.g., the sequencing platform 1045 described with respect to FIG. 10). This raw signal data represents the initial measurements or signals detected by the sequencing instrument, such as fluorescence intensities in Illumina sequencing, electrical current disruptions in Oxford Nanopore sequencing, or light pulses in PacBio sequencing. To make this data interpretable and usable for downstream analysis, it must undergo a process called base calling, where the raw signals are translated into nucleotide sequences. Base calling algorithms analyze the signal patterns, correlate them with nucleotide bases, and assign quality scores to each base to indicate the confidence level of the call. The base calling can be performed by the high-throughput sequencer, or may be transmitted to a server for the processing. Advanced base calling algorithms, including those powered by machine learning and deep learning techniques, can be used to improve the accuracy and efficiency of base calling. For example, the base calling algorithms can utilize neural networks (e.g., the neural network 601 illustrated in FIG. 6) to enhance the interpretation of electrical signals and fluorescence data, as well as providing real-time processing of the raw data.

The whole genome sequencing (WGS) can be conducted at varying levels of coverage, depending on the specific research or clinical requirements. The WGS can be performed at a medium to high coverage, for example, sequencing depths of about 10×-30× or about 30×-100×. In some embodiments, the WGS is performed at a sequencing depth of about 30×-40×, to balance data quality with resource efficiency. In some embodiments, WGS includes low-coverage whole genome sequencing (lcWGS). IcWGS involves sequencing the entire genome at a relatively low depth, typically between 1× to 10× coverage, where each base or base pair is sequenced only a few times. This method is cost-effective, faster, and less resource-intensive compared to high-coverage WGS. IcWGS can further include ultra-low coverage whole genome sequencing (ulcWGS), which sequences the genome at depths of less than 1× coverage (e.g., 0.4×). ulcWGS can be effective for detecting large-scale genomic variations, such as copy number variations (CNVs), aneuploidies, or other structural abnormalities, and is widely used in non-invasive prenatal testing (NIPT) to identify chromosomal anomalies in fetuses via cfDNA from maternal blood.

At block 1110, the raw data generated during the sequencing process is transmitted to a computing system (e.g., a GPU including at least one processor and a memory) for further processing, storage, or analysis. The computing system could be a local server connected to the high-throughput sequencing system or a cloud-based server accessible via a network (e.g., the server 1035 described with respect to FIG. 10). In some embodiments, the server is a GPU server. This transmission of raw data may occur over a secure network to ensure the confidentiality and integrity of the data, particularly when dealing with sensitive genetic information, such as patient-derived samples. Once received by the server, the raw data can undergo additional computational workflows, including base calling, alignment to a reference genome, filtering, variant calling, somatic mutation detection, or other bioinformatics analyses. The server may be a local server connected to the sequencer, or a cloud server that is part of an integrated cloud-based platform.

In some embodiments, the raw data generated during the sequencing process is transmitted to the server in real time. The term “real time” refers to the process of transmitting or processing data immediately as it is generated, without any significant delay (e.g, within several seconds or milliseconds). For example, the raw signal data, such as fluorescence intensities, electrical current disruptions, or light pulses, is sent directly from the sequencing instrument to the server as the sequencing run progresses. Real-time data transmission enables dynamic and concurrent processing, such as performing base calling, quality control, and preliminary analyses while the sequencing is still ongoing. Real-time data transmission provides faster overall processing times since computational workflows can begin without waiting for the entire sequencing run to complete. Additionally, real-time transmission facilitates remote monitoring of sequencing runs, enabling researchers or clinicians to observe data generation and ensure quality without being physically present near the sequencing instrument. In some embodiments, real-time workflows are further integrated with cloud-based platforms, allowing for scalable, high-speed data analysis and collaboration across multiple teams or locations.

At block 1115, the raw data is processed by the computing system on the server to generate candidate variants and associated feature values. This processing can be performed using the tools 1040 (e.g., through the preprocessing unit 1050 and the feature extractor 1055) described with respect to FIG. 10. The process involves obtaining sequence reads that are obtained from the raw data through base calling. The sequence reads can be determined by converting the raw signals (e.g., fluorescence intensities, electrical current disruptions, or light pulses) into nucleotide sequences with associated quality scores. The base-calling process can be performed before the raw data is transmitted to the server (in such instances, the raw data includes the sequence reads and quality scores), or performed on the server after the raw data is transmitted.

Next, the sequence reads are filtered based on a predetermined protocol to generate filtered sequence reads, ensuring that low-quality or ambiguous reads are excluded to improve the accuracy of downstream analyses. In some embodiments, the protocol is a machine learning-based filtering protocol that ensures that only “high-quality” sequence reads (e.g., the filtered sequence reads) are retained for downstream analysis, reducing the size of the dataset and computational burden compared to conventional methods that process larger volumes of raw data.

Once the filtered sequence reads are prepared, candidate variants are determined (e.g., by comparing the reads to a reference genome or other relevant datasets). Candidate variants may include single nucleotide polymorphisms (SNPs), insertions, deletions, or structural variations. For each candidate variant identified, a set of feature values is determined. These feature values may include metrics such as read depth, variant allele frequency, mapping quality, and surrounding sequence context. The feature values can be generated using both the filtered sequence reads and the original raw data, ensuring a comprehensive analysis that incorporates all available information.

In some embodiments, the predetermined protocol for filtering sequence reads includes various techniques designed to improve the accuracy of somatic mutation detection in WGS data. One such technique involves filtering out all short tandem repeats (STRs), which are repetitive DNA sequences prone to sequencing errors. Alternatively, STRs that fall outside of exons may be specifically filtered out, ensuring that the analysis focuses on regions more likely to harbor biologically significant mutations. Other filtering techniques may include removing low-quality reads based on PHRED scores, discarding reads with excessive mismatches or poor alignment scores relative to the reference genome, and excluding reads with ambiguous base calls (e.g., “N”). Additionally, reads originating from highly repetitive regions of the genome, such as centromeres or telomeres, can be filtered out to reduce false-positive variant calls. In some embodiments, the filtering protocols may also incorporate read-pair information, removing reads with abnormal insert sizes or orientation inconsistencies, as well as filtering out duplicate reads that may arise during library preparation or amplification. Strand-specific filtering can also be applied, ensuring that variants are supported by reads mapped to both the forward and reverse strands, thereby reducing strand bias. In some embodiments, reads with evidence of sequencing artifacts, such as those caused by polymerase slippage or mispriming, can also be flagged and removed. Finally, context-based filtering techniques, such as excluding variants in regions with low mappability or high GC content, can further refine the dataset and enhance the accuracy of downstream analyses.

Determining candidate variants based on the filtered sequence reads involves identifying genomic variations by comparing the high-quality reads obtained after filtering to a reference genome or other appropriate datasets. This process allows for the detection of variations such as single nucleotide polymorphisms (SNPs), insertions, deletions (indels), copy number variations (CNVs), and structural variations (SVs). The process begins by aligning the filtered sequence reads to the reference genome using advanced alignment algorithms, which ensure accurate mapping even in regions with complex or repetitive sequences. Once the sequence reads are aligned, candidate variants are detected by analyzing differences between the aligned reads and the reference genome. For example, mismatched bases between the sequence reads and the reference genome may indicate SNPs, while gaps in the alignment could signal insertions or deletions. Sophisticated variant callers, such as GATK (Genome Analysis Toolkit), FreeBayes, or VarScan, are commonly used to identify these candidate variants by applying statistical models to distinguish true genomic variants from sequencing errors or noise. To enhance accuracy, the variant detection process often incorporates additional contextual information, such as read depth, base quality scores, and mapping quality scores, to filter out potential false positives. Variants supported by a higher number of high-quality reads are more likely to be true positives. Strand bias is also evaluated to ensure that candidate variants are supported by reads from both forward and reverse strands, reducing the risk of artifacts caused by sequencing errors. In some embodiments, the candidate variants are determined using a trained machine learning model (e.g., the neural network 601 illustrated in FIG. 6). In some embodiments, at least 10,000, at least 100,000, or at least 1 million candidate variants are determined for a sample using WGS.

Once candidate variants are determined, feature values are also extracted for each candidate variant. The feature values correspond to a set of features that are specific metrics and attributes related to the candidate variants. The set of features may include a candidate variant unique identifier (UID) and various coverage and quality measures. For example, the features include tumor-specific data includes total coverage, counts of distinct nucleotide bases (A, C, G, T), mutant allele counts, and quality scores (sum, minimum, maximum, and average of PHRED quality scores). The set of features also tracks forward and reverse mutant pairs and distinct tumor coverage metrics, such as tags and pairs above a PHRED cutoff. In some embodiments, a mutation percentage calculated as the ratio of distinct pairs to distinct coverage is also included in the set of features. Mutation types (e.g., SBS, INS, DEL) and genomic coordinates (chromosome, start, end, base from/to) can also be included in the set of features. Additional features include whether the variant is germline (based on germline mutation percentage thresholds), its presence in SNP regions, and various normal coverage metrics, such as distinct raw and filtered coverage, mutant counts, distinct pairs, counts of fragments with mutant bases near read ends, and whether the variant is flagged for deletion or ignored. Table below provides an example of a set of features.

### Example Feature Table:

| Column | Name | Description |
|---|---|---|
| 1 | UID | Internal genomic variant unique |
| | | identifier |
| 2 | Coverage | Tumor total coverage |
| 3 | ACount | Number of distinct A bases at |
| | | this position |
| 4 | CCount | Number of distinct C bases at |
| | | this position |
| 5 | GCount | Number of distinct G bases at |
| | | this position |
| 6 | TCount | Number of distinct T bases at |
| | | this position |
| 7 | MutCount | Count of total mutant alleles |
| 8 | SumQualityScore | Sum of PHRED quality scores at |
| | | location (why would anyone |
| | | care what this is?) |
| 9 | MinQuality Score | Min PHRED score at location |
| 10 | MaxQuality Score | Max PHRED score at location |
| 11 | AverageQuality Score | Average PHRED score at |
| | | location |
| 12 | Forward | Forward total mut pairs |
| 13 | Reverse | Reverse total mut pairs |
| 14 | DistinctCoverage | Tumor distinct coverage |
| 15 | DistinctTags | Distinct tumor tags with this |
| | | mutant above PHRED cutoff |
| 16 | DistinctPairs | Distinct tumor pairs with this |
| | | mutant above PHRED cutoff |
| 17 | MutPct | Distinct “tags” considers only |
| | | the read in question when |
| | | determining uniqueness, while |
| | | “pairs” considers both the tag |
| | | and its mate when determining |
| | | uniqueness As a result, distinct |
| | | “pairs” are generally a |
| | | higher number and also the one |
| | | you want to look at. “Tags” |
| | | should only be higher when you |
| | | have a bunch of unpaired reads |
| | | mutpct = |
| | | distinctpairs/distinctcoverage |
| | | MutPct Notes |
| 18 | MutType | SBS, INS, DEL, etc |
| 19 | Chrom | Chromosome |
| 20 | Start | Start position |
| 21 | End | End position |
| 22 | BaseFrom | “From” base |
| 23 | BaseTo | “To” base |
| 24 | Deleted | Not in ROI and ignore. 0 or 1. |
| 25 | SNP | Is in SNPs ROI bed? |
| 26 | NormalMutCount | Distinct normal tags with this |
| | | mutant above PHRED cutoff |
| 27 | NormalRawCoverage | Total normal coverage |
| 28 | NormalPhredCoverage | Total normal coverage above |
| | | PHRED cutoff (what we use) |
| 29 | NormalDistinctRawCoverage | Distinct normal coverage |
| 30 | NormalDistinctPhredCoverage | Distinct normal coverage above |
| | | PHRED cutoff (what we use) |
| 31 | GermlineNormMutPct | mutpct_n = (float) |
| | | conch_n.distinctpairs/(float) |
| | | conch n.distinctcoverage |
| 32 | Germline? | isgerm if mutpct_n > |
| | | min_mutpctgerm |
| 33 | TotalNormalMutCount | Count of distinct normal alleles |
| 34 | NormalDistinctMutPairs | Distinct normal pairs with this |
| | | mutant above PHRED cutoff |
| 35 | DistMutPairsEORA | number of fragments with a |
| | | mutant base w/in 10 bp of |
| | | the end of a read |
| 36 | DistMutPairsEORB | number of fragments with a |
| | | mutant base w/in 10 bp of |
| | | the end of a read |

Additional features may include read distributions, proper pairing percentages, masked reads, polyN sequences, GC/AT content, mapping quality scores, statistical scores quantifying relationships between mutations and non-mutated regions, metrics related to overlapping reads, average fragment sizes, read lengths, and metrics related to contextual genomic information.

In some embodiments, the set of features includes at least 20 features. The 20 features may be predetermined to include: (i) a count of unique instances where each nucleotide is observed at a specific position in the candidate variant, (ii) a count of sequence reads at a specific position in the candidate variant that show a mutated allele, (iii) a specific statistic of a quality score at a specific position in the candidate variant, (iv) a count of mutant allele pairs observed in forward strand reads at a specific position in the candidate variant, (v) a count of mutant allele pairs observed in reverse strand reads at a specific position in the candidate variant, (vi) a count of sequence reads at a specific position in the candidate variant that show a mutated allele above a cutoff quality score, and (vii) a mutation type. In some embodiments, the set of features includes at least 50 features, 60 features, 70 features, 80 features, 90 features, or 100 features.

The feature values for each candidate variant are then stored in a specific data structure, which is selected to optimize data organization, processing efficiency, and accessibility for downstream applications. Examples of such data structures include a table, which can be implemented in various forms like a relational database table or a hash table for efficient querying and indexing; a multi-dimensional array, which allows for compact and computationally efficient storage of complex datasets; a linked list, which is particularly useful for dynamic datasets that require frequent updates or reordering; or a matrix, which organizes data into rows and columns for high-speed computational calculation. Alternatively, feature values may be stored in a DataFrame, a versatile and widely used data structure in data analysis frameworks like pandas, offering powerful tools for filtering, grouping, and statistical computations. Other formats include JSON (JavaScript Object Notation), a lightweight and hierarchical format ideal for data exchange and integration with web-based applications, or spreadsheets, which are user-friendly and allow for manual inspection and editing of data. For large-scale sequencing workflows, feature values may also be stored in specialized file formats designed for genomic data, such as VCF (Variant Call Format) or HDF5, which are optimized for handling large datasets and ensuring compatibility with bioinformatics tools. In some embodiments, the table used to store feature values may be a relational database table, such as those implemented in SQL-based systems, where relationships between data points can be defined and queried efficiently. Alternatively, a hash table may be used for rapid data retrieval, particularly in scenarios where candidate variants need to be accessed using unique keys, such as genomic positions or variant identifiers. These data structures ensure that the feature values are easily accessible and can be seamlessly integrated into downstream processing pipelines, including variant annotation, prioritization, and data visualization.

At block 1120, the feature values generated from the filtered sequence reads are processed by the computing system using a trained machine learning model to accurately identify somatic mutations. The trained machine learning model is first loaded into the memory of the computing system, and memory and computational resources are allocated by the computing system to execute the trained machine learning model. In some embodiments, the memory allocation is dynamic memory management for model execution, and the computational resource allocation assigns processing tasks to specialized accelerators of the computing system. After the allocation, the feature values generated at block 1115 are input into the trained machine learning model, and output data (including the somatic mutations and associated metadata) are provided by the trained machine learning model.

This process involves leveraging advanced computational algorithms to analyze the comprehensive set of feature values associated with each candidate variant. The machine learning model can be trained on high-confidence datasets containing labeled somatic and germline variants, applying pattern recognition and predictive analytics to distinguish true somatic mutations from noise, sequencing artifacts, and germline variants.

The machine learning model may employ supervised learning techniques, such as support vector machines (SVM), random forests, or neural networks, to classify variants based on their feature values. For example, the model can be trained to recognize the unique characteristics of somatic mutations, such as their lower allele frequency in tumor samples compared to germline variants or their presence in regions with specific mutational signatures. Additionally, the model may incorporate ensemble learning methods to combine predictions from multiple algorithms, enhancing the accuracy and robustness of the mutation detection process.

In some embodiments, techniques disclosed herein include training a machine learning model to select somatic mutations from candidate variants. The training begins with the acquisition of high-quality training data. This data is derived from sequencing data of matched tumor-normal samples. Each sample may include labeled variants, with each variant being classified as either a somatic mutation or a non-somatic mutation. Along with these labels, the training data also includes a comprehensive set of feature values for each variant. These feature values may include attributes in the example feature table above.

Once the training data is prepared, it is divided into two or more subsets: a training set, a validation set, and a test set. The training set is used to train the machine learning model by inputting the feature values and corresponding labels, allowing the model to learn patterns and relationships within the data that minimize misclassification errors. The model iteratively adjusts its internal parameters based on the training data to optimize its predictions. The validation set, on the other hand, is used to evaluate the model's performance on unseen data, ensuring that the model generalizes well and is not overfitting to the training data. This process allows for a clear assessment of the model's accuracy and reliability in identifying somatic mutations.

If the model does not meet a predetermined performance standard during validation—such as achieving a specific accuracy, precision, recall, or F1 score—hyperparameter tuning is performed. Hyperparameters are adjustable settings that control the behavior of the machine learning model, such as the learning rate, the number of layers in a neural network, or the number of decision trees in a random forest. By modifying these hyperparameters and iteratively retraining and validating the model, the performance is systematically improved. This iterative process continues until the model meets or exceeds the predetermined standard, ensuring that it is well-optimized for the task of somatic mutation detection. Once the model achieves the desired performance threshold, the trained machine learning model is finalized (testing using the test set to determine accuracy and specificity) and output for use.

This processing step can be performed on the mutation detection platform 1015, which operates on the server 1035 described with respect to FIG. 10. The server provides the computational power necessary to handle the large-scale data generated during WGS, enabling efficient and scalable analysis. The mutation detection platform integrates the machine learning model with preprocessing tools (e.g., tools 1040), such as alignment and variant calling, and post-processing pipelines, such as annotation and prioritization, to deliver a seamless end-to-end solution for somatic mutation detection.

The results of the machine learning analysis include a refined list of somatic mutations, each annotated with its corresponding feature values and/or confidence scores generated by the model. These somatic mutations are stored in a structured format, such as a variant call format (VCF) file, and are ready for downstream analysis, including functional annotation, pathway enrichment, tumor burden determination, ctDNA level/score determination, minimal residual disease (MRD) status evaluation, and other clinical interpretation.

In some embodiments, the trained machine learning model used for somatic mutation detection is a random forest model comprising at least 500 or at least 1,000 decision trees. A random forest model is an ensemble learning method that combines the predictions of multiple decision trees to improve classification accuracy, reduce overfitting, and enhance the robustness of the analysis. Each decision tree in the random forest is trained on a random subset of the feature values and data samples, which ensures diversity among the trees and helps the model capture complex patterns in the data. By constructing hundreds or thousands of decision trees, the random forest model aggregates their individual predictions through a majority voting mechanism, yielding a consensus decision for each candidate variant. This approach mitigates the impact of noise or biases present in individual trees, resulting in a more accurate and reliable classification of somatic mutations. Random forest models are also capable of ranking the importance of input features, providing insights into which attributes (e.g., allele frequency or mapping quality) contribute most to the classification of somatic mutations. This feature importance analysis can be used to refine the model further, streamline the feature set, or improve interpretability for researchers and clinicians.

In some embodiments, a multi-tiered memory caching system is implemented in the computing system to optimize retrieving and processing the raw data and feature values. The multi-tiered memory caching system can further include a priority queue for candidate variants, and the candidate variants are stored based on a confidence level (e.g., the likelihood that a candidate variant is a true variant and/or the likelihood that the candidate variant is a somatic variant) generated by the trained machine learning model.

At block 1125, the somatic mutation information generated through the trained machine learning model (together with other processing information) is transmitted from the computing system to an end device (e.g., a client device 1005 described with respect to FIG. 10). The end device is communicatively connected to the computing system via the network. This transmission allows the processed results to be accessed and utilized by researchers, clinicians, or other authorized users. The somatic mutation information may include data such as the genomic coordinates of mutations (e.g., chromosome, start, and end positions), mutation types (e.g., single nucleotide substitutions, insertions, deletions), allele frequencies, quality scores, and any functional annotations, such as gene impact, clinical significance, or associated pathways. The transmission process can be performed over a secure network (e.g., a network 1020 described with respect to FIG. 10) to ensure the confidentiality and integrity of sensitive genomic data, particularly when the somatic mutations are derived from patient samples. Depending on the system architecture, the data may be transmitted in various formats, such as Variant Call Format (VCF), JSON, or other structured file types optimized for downstream analysis or visualization. In some embodiments, the somatic mutation information is transmitted to a cloud-based platform or a web-based interface, enabling real-time access, remote collaboration, and seamless integration with data analysis pipelines or electronic health records.

In some embodiments, the end device (e.g., client device 1005) may include a variety of hardware, such as desktop computers, laptops, tablets, or smartphones, equipped with software tools for viewing, analyzing, or interpreting the transmitted data. For example, the device could host a graphical user interface (GUI) that allows users to visualize somatic mutation data in the context of genomic features, filter the results based on specific criteria (e.g., mutation type, frequency, or clinical relevance), and generate reports for research or clinical decision-making. The somatic mutation information thus is accessible to stakeholders in a timely and user-friendly manner, facilitating downstream applications such as cancer diagnostics, personalized treatment planning, biomarker discovery, or population-level genomic studies.

At block 1130, a report regarding the somatic mutations is displayed on the end device, providing the user with a detailed and organized presentation of the identified mutations and their associated data. This report (e.g., the report illustrated in FIG. 8) may include key information about each somatic mutation, such as its genomic coordinates (e.g., chromosome, start, and end positions), mutation type (e.g., single nucleotide substitution, insertion, deletion), allele frequency, read depth, and quality scores. Additionally, the report may feature functional annotations, including the affected gene, protein impact (e.g., missense, nonsense, or frameshift mutations), clinical significance (e.g., pathogenic, likely benign), and any associations with known diseases or drug responses. The report can be tailored to the specific needs of the user, enabling customization of the displayed information. For instance, clinicians may focus on clinically actionable mutations relevant to patient care, while researchers may prioritize mutations of interest for experimental follow-up. The report may also include summary statistics, such as the total number of somatic mutations detected, mutation frequency distributions, and the proportion of mutations within specific genomic regions (e.g., exons, introns, promoters).

The report is typically presented in a user-friendly format, which could range from a tabular layout to an interactive graphical interface. In some embodiments, the report includes visualization tools, such as bar charts, scatter plots, or genome browsers, to help users interpret the data more effectively. For example, the report may allow users to view mutations mapped onto the reference genome, highlighting hotspots of mutation activity or regions with significant structural changes. The report may also incorporate filtering and sorting options, enabling users to refine the displayed results based on criteria like mutation type, allele frequency, or clinical relevance.

In some embodiments, since the identified somatic mutations are specific to a patient's tumor, clinicians and researchers can gain insights into the genetic drivers of the disease, enabling the design of targeted therapies. For example, mutations in key oncogenes (e.g., KRAS, EGFR, or BRAF) or tumor suppressor genes (e.g., TP53 or BRCA1/BRCA2) can reveal vulnerabilities in tumor cells that can be exploited by specific drugs, such as tyrosine kinase inhibitors or PARP inhibitors. Additionally, somatic mutations can guide the use of immunotherapy by identifying tumor-specific neoantigens, which are proteins uniquely expressed by mutated genes in cancer cells. These neoantigens can help predict a patient's response to immune checkpoint inhibitors or inform the development of personalized cancer vaccines.

In some embodiments, the somatic mutations are integrated with external clinical databases and knowledge repositories to provide real-time contextual annotations and a recommendation for a personalized treatment plan (e.g., including therapeutic decision-making). The contextual annotations may include drug-gene interaction information, clinical trial eligibility, and/or prognostic insights to provide the recommendation for the personalized treatment plan. By synthesizing these annotations with patient-specific clinical data, techniqus disclosed herein further enable healthcare professionals to make informed therapeutic decisions tailored to the patient's unique molecular profile. This integration also enhances precision medicine by ensuring that treatment strategies are both timely and personalized.

In some embodiments, the somatic mutation data can be aggregated to determine a ctDNA level or generate a ctDNA score. For example, for each identified somatic mutation, a variant allele fraction (VAF), which represents the proportion of reads supporting the mutation compared to the total number of reads at that position, may be generated. The VAFs provide an estimate of the abundance of tumor-derived DNA fragments carrying the mutation in the sample. These individual VAFs can then be aggregated across all identified somatic mutations within the sample to estimate the overall circulating tumor DNA (ctDNA) level. This aggregation may provide a quantitative measure of the tumor-derived genetic material present in the sample.

To aggregate the VAFs of the detected somatic mutations and estimate the overall ctDNA level, several approaches can be used. One method is the summation of VAFs, where the VAFs of all somatic mutations are summed to generate a cumulative ctDNA score. Alternatively, a weighted aggregation can be performed, where VAFs are weighted based on additional factors such as the genomic context, mutation type, or confidence scores assigned during the variant calling process. Another approach is normalization, where the aggregated VAFs are normalized by the total number of sequence reads or specific genomic regions to account for potential technical variability or differences in sequencing depth. Once the aggregated ctDNA level is determined, it may be compared to a reference or baseline dataset to assess its clinical significance. For instance, a higher ctDNA level may indicate a greater tumor burden or disease progression, while a decrease in ctDNA levels over time may reflect a positive response to treatment or remission. This comparison provides valuable insights into the patient's disease state and treatment efficacy, making ctDNA analysis a powerful tool for monitoring tumor dynamics and guiding clinical decision-making.

In some embodiments, the identified somatic mutations are processed using a second trained machine learning model to identify a tumor metric (e.g., a circulating tumor DNA (ctDNA) level or a tumor/cancer status). The second trained machine learning model may be further configured to identify a personalized treatment plan for the subject from whom the sample is obtained. The processing can be performed by the computing system where the trained machine learning model is deployed, or may be performed at the end device. In some embodiments, the second trained machine learning model is loaded into the memory of the computing system (or the end device, depending on the system setting), and memory and computational resources are allocated by the system to execute the second trained machine learning model. The somatic mutations are then into the second trained machine learning model, and output data, including the tumor metric and/or the personalized treatment plan, are generated by the second trained machine learning model. In some embodiments, the report includes the tumor metric and/or the personalized treatment plan.

In some embodiments, the second trained machine learning model integrates identified somatic mutations with other patient-specific, clinical, or biomarker data to provide prognostic or predictive insights. For example, the second trained machine learning model may combine somatic mutation data, such as the presence of specific driver mutations, with clinical variables like tumor stage, patient age, or treatment history to generate a comprehensive risk assessment. This integration allows for a more nuanced understanding of a patient's disease trajectory, enabling predictions about outcomes such as overall survival, likelihood of recurrence, or response to specific therapies. The machine learning model may also incorporate biomarker data, such as protein expression levels, epigenetic modifications, or circulating tumor DNA (ctDNA) levels, to refine its predictions. By analyzing the relationships between somatic mutations, biomarkers, and clinical features, the model can identify patterns or correlations that are not immediately apparent through traditional analysis. For example, the presence of certain mutations in combination with elevated ctDNA levels may indicate an aggressive tumor phenotype and guide clinicians toward more intensive treatment options.

In addition to prognostic insights, the second trained machine learning model may provide predictive information relevant to therapeutic decision-making. For instance, it may predict how a patient will respond to targeted therapies based on the presence of actionable mutations, such as EGFR mutations in non-small cell lung cancer or BRCA1/2 mutations in breast or ovarian cancer. The integration of mutation data with pharmacogenomic databases can further enhance the model's predictive accuracy by linking mutations to known drug response profiles or resistance mechanisms. Furthermore, the second trained machine learning model may leverage longitudinal patient data to provide dynamic predictions over time. By analyzing changes in somatic mutations or biomarker levels across multiple time points, the model can detect early signs of disease progression or treatment resistance, supporting timely clinical interventions. For example, a rising ctDNA level combined with newly identified mutations associated with resistance may prompt a switch in therapy before clinical symptoms manifest.

In some embodiments, the second machine learning model is configured to incorporate real-time clinical and/or genomic data updates to dynamically fine-tune its output generation. This capability ensures that the second trained machine learning model remains adaptive and responsive to the most current and relevant information, thereby enhancing its accuracy and applicability in dynamic environments. For example, the system may identify FDA-approved therapies or experimental treatments relevant to the detected mutations, highlight clinical trials the patient may qualify for, and provide insights into disease progression or risk based on the mutations. For example, in the context of somatic mutation detection and precision medicine, the second trained machine learning model can continuously integrate real-time updates from sources such as new sequencing data, patient health records, or evolving clinical guidelines. These updates might include recent genomic variants identified during sequencing, up-to-date patient-specific biomarkers, or the latest drug-gene interaction data. By dynamically refining its computational process based on these inputs, the second trained machine learning model can prioritize the most clinically relevant mutations, further improve sensitivity in detecting rare variants, and provide actionable insights faster.

In some embodiments, this real-time adaptability is particularly helpful in scenarios like personalized treatment planning, where the integration of new data (e.g., a patient's recent response to therapy or emerging resistance markers) can significantly influence decision-making. The second trained machine learning model's ability to continuously update and optimize its performance ensures that healthcare professionals receive the most accurate and timely information, facilitating better outcomes in precision medicine and personalized treatment strategies.

Beyond treatment design, somatic mutations can also be used for understanding tumor evolution and monitoring disease progression. By analyzing the clonal architecture of somatic mutations, researchers can identify subclonal populations within a tumor, providing insights into tumor heterogeneity and treatment resistance mechanisms. Somatic mutations can also serve as biomarkers for minimal residual disease (MRD), enabling real-time tracking of cancer recurrence through liquid biopsies that detect ctDNA in the bloodstream. This also allows for earlier intervention and adjustment of treatment plans based on the patient's dynamic tumor profile.

For example, a presence or absence of specific mutations within a predefined set of somatic mutations may be determined or identified. Each mutation in the set may correspond to a gene or pathway implicated in disease progression or therapeutic response. Based on the presence or absence of these mutations, a genotype-directed therapy is identified for the subject. This process leverages pharmacogenomic databases and clinical guidelines to match actionable mutations with targeted therapies or combinations of therapies. For example, the presence of EGFR mutations may guide the use of EGFR inhibitors, while the absence of mutations indicating drug resistance may confirm eligibility for specific treatments.

In some embodiments, the identified somatic mutations are evaluated for potential associations with inherited genetic conditions. This step involves distinguishing somatic mutations, which occur in the tumor and are not inherited, from germline mutations, which are present in all cells and passed down through generations. By cross-referencing somatic mutations with known inherited disease databases, such as ClinVar or OMIM, clinicians can assess whether certain mutations are indicative of hereditary syndromes, such as Lynch syndrome or BRCA1/2-associated hereditary breast and ovarian cancer. This information can be used for determining whether genetic counseling or additional testing for inherited conditions is necessary for the patient or their family members.

In some embodiments, a circulating tumor DNA (ctDNA) level can be determined based on the identified somatic mutation information. In some embodiments, the ctDNA level is determined using a separate clinical assay or pipeline. The aggregated ctDNA level provides a measure of tumor burden and can be used to evaluate the patient's response to treatment. For instance, a significant reduction in ctDNA levels over time may indicate effective therapy, while stable or rising levels may suggest resistance or progression. Additionally, ctDNA levels, in combination with remission status, help monitor minimal residual disease (MRD) and assess whether the patient has achieved complete or partial remission. This non-invasive approach allows for dynamic monitoring of disease and supports evidence-based treatment adjustments.

### B. Wet-Lab and In-Silico Processes

### Sample Collection

In the exemplary processing workflow 1100, a biological sample is obtained from a subject for analysis. The sample, also referred to as a biological sample, can include either a cell-containing liquid or tissue. Examples of such biological samples include, but are not limited to, amniotic fluid, tissue biopsies, blood, blood cells, bone marrow, fine needle biopsy samples, peritoneal fluid, plasma, pleural fluid, saliva, semen, serum, tissue or tissue homogenates, and frozen or paraffin-embedded sections of tissue. Methods for collecting these samples encompass a variety of techniques, such as obtaining biofilms, performing aspirations, collecting tissue sections, using swabs, drawing blood or other bodily fluids, and conducting surgical or needle biopsies, among others. The biological sample collected from the subject may serve as a source of nucleic acids, such as DNA and/or RNA, in both natural and synthetic forms. For example, plasma samples collected from the subject by drawing blood can provide circulating cell-free DNA or RNA for downstream molecular analyses. These versatile sample types and collection methods enable the comprehensive study of genomic material, supporting diverse applications in research, diagnostics, and personalized medicine.

As an example of sample collection, a whole blood sample can be obtained from the subject using venipuncture or other standard methods known in the art. To isolate plasma, an anticoagulant is added to the blood sample to prevent clotting, and the sample is centrifuged at sufficient speed to separate the plasma from the cellular components. The resulting plasma sample contains nucleic acids, such as cell-free DNA (cfDNA) or circulating tumor DNA (ctDNA), which can be used for downstream molecular analysis. The remaining fraction, separated from the plasma, comprises the cellular components of blood, including white blood cells (e.g., monocytes, lymphocytes, neutrophils, eosinophils, basophils, and macrophages), red blood cells (erythrocytes), platelets, and a buffy coat fraction that contains leukocytes and thrombocytes. While plasma is highlighted as an exemplary biological sample, other sample types that contain cell-free DNA can also be utilized. Examples include sputum, saliva, cerebrospinal fluid, surgical drain fluid, urine, cyst fluid, and similar biological fluids. These diverse sample types provide flexibility in obtaining nucleic acids for genomic studies, supporting a wide range of applications in research, diagnostics, and personalized medicine.

In some embodiments, the subject is healthy or found not to have cancer. As such, the biological sample collected from the subject (e.g., plasma) comprises cfDNA originating from non-cancerous sources. For example, the biological sample comprising cfDNA may include DNA fragments from cells undergoing apoptosis as part of the normal physiological process of the cell cycle, fetal DNA in the case of pregnant individuals, transplant DNA derived from donor organs in transplant recipients, microbial DNA from commensal or pathogenic microorganisms, or any other form of cfDNA that is non-cancerous. In the context of organ transplantation, cfDNA analysis can be used to monitor the presence and levels of donor-derived cfDNA (dd-cfDNA) in the recipient's blood. Elevated levels of dd-cfDNA may indicate organ rejection, providing a non-invasive tool for early detection and monitoring of transplant health. In other instances, the subject is diagnosed with cancer or is receiving treatment for cancer. Accordingly, the biological sample collected from the subject (e.g., plasma) comprises cfDNA originating from cancerous sources (e.g., tumor cells) in addition to non-cancerous sources.

Once the biological sample (e.g., plasma) is collected from the subject, the sample is prepared for DNA isolation. Various methods are well-established in the art for isolating DNA from biological samples. One common approach involves using commercially available reagent kits that include necessary components such as tubes, DNA extraction reagents, and protocols. These kits may also provide tools for subsequent library preparation steps, including probes for hybrid capture and reagents for fragmentation, adapter ligation, purification, or isolation of DNA. By utilizing these kits or other techniques known in the field, a sample containing DNA can be successfully obtained for downstream applications. Alternative methods for isolating or extracting DNA from a sample typically involve disruption and lysis of the starting material, followed by the removal of proteins and other contaminants, and finally recovery of the DNA. Cell lysis can be achieved using chemical methods (e.g., detergents, hypotonic solutions, enzymatic treatments), physical methods (e.g., French press, sonication), or electrolytic lysis methods. The removal of proteins is often performed using digestion with proteinase K, followed by techniques such as salting-out, organic extraction, gradient separation, or binding of DNA to a solid-phase support, such as anion-exchange or silica-based technologies. DNA recovery is generally achieved through precipitation using ethanol or isopropanol.

The choice of DNA isolation method depends on several factors, including the sample type (e.g., tissue, cells, or low-concentration cell-free DNA), the required yield and molecular weight of the DNA, the purity needed for downstream applications, and considerations of time and cost. The resulting isolated DNA may include whole genomic DNA, circulating cell-free DNA (cfDNA), circulating tumor DNA (ctDNA), mitochondrial DNA, circular DNA, or other forms of DNA.

The amount of DNA isolated from a sample depends on factors such as the type, size, and quality of the sample. For example, plasma samples can yield at least 2-10 ng of cfDNA, although this can vary. In some instances, to ensure sufficient quantities of cfDNA (e.g., at least 2 ng or 5 ng) are obtained, multiple whole blood samples may be collected from the patient, and the DNA extracted from these samples is pooled. For example, at least two 10 mL volumes of whole blood may be collected from the patient to obtain sufficient plasma for isolating at least 10 ng of cfDNA.

In some embodiments, when it is determined that the amount of nucleic acid obtained from a sample is insufficient for analysis, amplification techniques may be employed to increase the quantity of nucleic acid. Amplification refers to the process of generating additional copies of a specific nucleic acid sequence, enabling downstream applications that require higher concentrations of DNA or RNA. This process is commonly performed using polymerase chain reaction (PCR) or other amplification technologies known in the art (e.g., Dieffenbach and Dveksler, PCR Primer: A Laboratory Manual, 1995, Cold Spring Harbor Press, Plainview, NY). PCR is a widely utilized method developed by Kary B. Mullis (U.S. Pat. Nos. 4,683,195 and 4,683,202, hereby incorporated by reference), enables the exponential amplification of a target nucleic acid sequence without the need for cloning or extensive purification. The technique involves repeated cycles of denaturation, annealing of primers, and extension by a DNA polymerase enzyme, resulting in the rapid generation of millions of copies of the target sequence. PCR and related technologies are indispensable for applications such as whole genome sequencing, mutation detection, gene expression analysis, and clinical diagnostics, particularly when starting material is limited.

### Whole Genome Sequencing

The nucleic acid sample is sequenced using any suitable whole genome sequencing (WGS) methods. In some cases, nucleic acids may be amplified prior to sequencing to ensure sufficient material for analysis. Sequencing data obtained from WGS comprises sequence reads, which are the foundational units for subsequent genomic analysis.

### (a) DNA Fragmentation and Library Preparation

After DNA isolation, the next step involves preparing the DNA for sequencing, beginning with fragmentation. In some instances, the isolated DNA is fragmented into a collection of shorter double-stranded DNA target fragments. Fragmentation can be performed using physical methods (e.g., acoustic shearing or sonication) or enzymatic approaches (e.g., DNase I digestion) to generate fragments within specific size ranges. Examples of fragment size ranges include approximately 25-100 base pairs (bp), 25-150 bp, 50-200 bp, 25-200 bp, 50-250 bp, 25-250 bp, 50-300 bp, 25-300 bp, 50-500 bp, 25-500 bp, 150-250 bp, 100-500 bp, and 200-500 bp. In some cases, the DNA may remain unfragmented, such as with circulating cell-free DNA (cfDNA) samples (or can be treated as “already-fragmented” DNA).

Using the DNA fragments (or the “already-fragmented” cfDNA) prepared as described above, a DNA library is constructed. A DNA library is a collection of polynucleotide molecules (e.g., nucleic acid samples) that have been prepared, assembled, or modified for specific purposes. These purposes include, but are not limited to, immobilization on a solid phase (e.g., a solid support, flow cell, or bead), enrichment, amplification, cloning, detection, and nucleic acid sequencing. DNA libraries can be prepared either prior to or during the sequencing process. Libraries may be constructed through targeted or non-targeted preparation processes, depending on the sequencing objectives.

DNA library preparation involves several steps, which may include end repair, A-tailing, adapter ligation, amplification, or a combination of any of these steps. The goal of DNA library preparation is to create sequencing-ready samples for single-read sequencing, paired-end sequencing, or multiplexed sequencing. Initially, nucleic acids (fragmented or unfragmented) undergo end repair via a fill-in reaction, exonuclease treatment, or a combination of these methods to generate blunt-end DNA fragments. These blunt ends can then be extended by the addition of a single nucleotide, which is complementary to a single-nucleotide overhang on the 3′ end of an adapter or primer. For example, if A-tailing is used, an adenosine (A) nucleotide is added to the 3′ end of the DNA. Once the single-nucleotide overhang is added, an adapter oligonucleotide is ligated onto the DNA fragments, typically using an enzymatic ligation process, such as with T4 DNA ligase.

Adapter oligonucleotides serve several important functions. They are often complementary to anchors within the flow cell, enabling immobilization of the DNA library to a solid support during sequencing. Additionally, adapters may include one or more sequencing primer hybridization sites (e.g., regions complementary to universal sequencing primers), sample identifiers (IDs) for tracking nucleic acids from different samples, and barcodes (e.g., single-molecule barcodes or molecular barcodes) that allow for tracking individual DNA molecules, particularly when amplification occurs prior to sequencing.

In some instances, amplification of the DNA library or its components is performed to ensure sufficient DNA for sequencing. Amplification can be achieved using polymerase chain reaction (PCR)-based methods, thermocycling protocols, isothermal amplification, or rolling circle amplification. Amplification may occur either before or after immobilizing the DNA library on a bead or solid support, such as in a flow cell. Solid-phase amplification, for example, involves hybridizing the DNA library to anchors within the flow cell under suitable conditions. During this process, the amplified products are synthesized by an extension reaction that is initiated from an immobilized primer. Solid-phase amplification is analogous to conventional solution-phase amplification but differs in that at least one primer is immobilized on a solid support. In certain embodiments, modified nucleic acids (e.g., those with adapters or barcodes) are amplified to enhance sequencing efficiency. The prepared DNA library, whether amplified or not, is then ready for sequencing, enabling the generation of high-quality data for downstream genomic analysis.

### (b) Whole Genome Sequencing

The library-prepped nucleic acids (e.g., cfDNA) are sequenced using a sequencing platform (e.g., the sequencing platform @ 1045 described with respect to FIG. 10) capable of processing nucleic acids. Examples of sequencing technologies include, without limitation, NovaSeq, HiSeq, Genome Analyzer IIx, MiSeq, HiScanSQ, 454 DNA sequencer, GS FLX+, GS Junior System, OLID next-generation sequencing platform, Ion PGM System, Ion Proton System, Ion S5, Ion S5xl, CEQ 8000, RS system, Sequel system, nanopore sequencers, DNBSEQ-G50, DNBSEQ-G400, DNBSEQ-T7, Ultima Genomics UG100, and others.

Depending on the method or application, a full or substantially complete sequence of the nucleic acids may be obtained, while in certain cases, partial sequences may also suffice.

Any suitable nucleic acid sequencing method can be employed. Non-limiting examples include first-generation sequencing methods, such as Maxam-Gilbert or chain-termination (Sanger) sequencing; sequencing by synthesis; sequencing by ligation; sequencing by mass spectrometry; or microscopy-based techniques, such as transmission electron microscopy (TEM) or atomic force microscopy (AFM). High-throughput sequencing methods, also referred to as next-generation sequencing (NGS), are particularly advantageous for their ability to sequence clonally amplified DNA templates or single DNA molecules in a massively parallel fashion. Second- and third-generation sequencing technologies, which enable large-scale sequencing with enhanced speed and accuracy, are frequently employed for whole genome sequencing (WGS) workflows. In some embodiments, a non-targeted approach is used, allowing for random sequencing, amplification, or capture of most or all nucleic acids in a sample.

In certain embodiments, WGS is performed on the prepared DNA library samples. This process involves amplification of DNA on a solid surface using fold-back PCR and anchored primers. Genomic DNA is first fragmented, and adapters are ligated to the 5′ and 3′ ends of the fragments. The DNA fragments are then immobilized on the surface of flow cell channels, where they undergo bridge amplification to form clonal clusters. Each cluster contains approximately 1,000 copies of single-stranded DNA molecules derived from the same template. During sequencing by synthesis, primers, DNA polymerase, and four fluorophore-labeled, reversibly terminating nucleotides are used to sequentially incorporate bases into the growing DNA strand. After each nucleotide incorporation, a laser excites the fluorophores, and an image is captured to record the identity of the incorporated base. The 3′ terminators and fluorophores are removed, and the cycle is repeated. This iterative process generates high-quality sequencing data. This sequencing methodology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which is incorporated by reference in its entirety.

Sequencing methods, such as WGS, generate vast quantities of sequence reads, which are short nucleotide sequences produced by the sequencing process. Reads can either be “single-end reads” derived from one end of a nucleic acid fragment, or “paired-end reads” generated from both ends of the fragment. The length of these reads depends on the sequencing technology used and can range from tens to hundreds of base pairs (bp). For instance, sequence reads may have a mean, median, average, or absolute length ranging from about 15 bp to about 1,000 bp. Specific examples include lengths of about 15 bp, 20 bp, 50 bp, 150 bp, 300 bp, 500 bp, or any integer value between 15 bp and 1,000 bp.

The sequence reads, along with their corresponding quality scores, are typically stored in files such as FASTQ or FASTA. FASTQ files contain both the nucleotide sequences and their associated PHRED quality scores, which provide a measure of confidence in the accuracy of each base call. The number of reads generated per sample can vary depending on the type of sequencing performed. For example, plasma samples containing cfDNA can generate approximately 800 million to 2 billion reads per sample, enabling highly detailed analysis of the genetic material. These large datasets form the foundation for downstream bioinformatics workflows, including alignment, variant detection, and genome assembly.

### Bioinformatics Workflow

In some embodiments, sequence reads are generated, obtained, assembled, manipulated, transformed, processed, and/or provided by a preprocessing subsystem. The preprocessing subsystem may be part of any suitable machine or apparatus capable of determining the sequence of nucleic acids using established sequencing technologies. In addition to sequencing, the subsystem may perform tasks such as aligning, assembling, fragmenting, complementing, reverse complementing, and/or error-checking (e.g., error-correcting sequence reads).

As described, the outputs of sequencing are typically stored in FASTQ files, which contain all sequence reads for a single sample. Demultiplexing, which involves sorting pooled library samples from a single flow cell lane into individual FASTQ files, may be used to generate the sequence reads. For example, in a typical WGS sequencing run, multiple library samples (e.g., 4, 12, or 16) are pooled and loaded onto a single lane of a sequencing flow cell. Unique barcodes can be optionally used to distinguish one sample from another, enabling the separation of each sample into its respective FASTQ file during demultiplexing.

The alignment of reads to a reference genome involves mapping the sequence reads to specific regions of the reference genome, such as chromosomes or portions thereof. This process generates “counts,” representing the number of reads aligned to a given region. In some embodiments, post-alignment BAM files are generated, which may include the aligned read information and/or the corresponding counts. A variety of mapping and alignment methods can be used to perform this step, including algorithms, software, and programs such as BLAST, BLITZ, FASTA, BOWTIE (versions 1 and 2), ELAND, MAQ, PROBEMATCH, SOAP, BWA, or SEQMAP, as well as combinations or variations of these tools. Due to the large data size, alignments have to be performed computationally using tools alignment tools. Alignments may range from perfect matches (100%) to partial matches (e.g., 75-99% identity), with some alignments allowing for mismatches (e.g., 1-5 mismatches). Additionally, alignments can be performed on either strand (sense or antisense) and may involve reverse complements of sequences. The results of the alignment process are stored in alignment files, such as BAM files.

As part of quality control, alignment files may be filtered to remove non-primary alignment records, reads mapped to improper pairs, and reads with more than six edits. Individual bases with PHRED base quality scores below specific thresholds (e.g., less than 30 for tumor samples or less than 20 for normal samples) are excluded.

Candidate variations between the sample and the reference genome are identified through a process known as variant calling. Variants represent alterations in the DNA sequence that differ from the reference and can be classified as benign, likely benign, variants of unknown significance (VUS), likely pathogenic, or pathogenic. Variants may include germline variants (present in all cells of the body) and somatic variants (arising during an individual's lifetime, such as in cancer). Examples of variants include small sequence variants (less than 50 base pairs) such as single nucleotide variants (SNVs), single nucleotide polymorphisms (SNPs), and small structural variants (SVs) like deletions or insertions (indels). Larger SVs, such as chromosomal rearrangements (e.g., translocations, inversions) and copy number changes, may also be detected. SNVs and SNPs can result in synonymous changes (no change in the encoded amino acid), missense changes (altering the amino acid), or nonsense changes (introducing a stop codon). Variants can occur in both coding and non-coding regions of the genome and are detectable through WGS, which provides a broader scope of analysis compared to targeted gene panels.

Variant calling involves specialized tools that analyze aligned sequencing data alongside the reference genome to identify potential mutations, such as single-base substitutions and small indels. These tools evaluate candidate variants by scoring metrics such as read depth, allele frequency, and mapping quality, applying thresholds to distinguish true mutations from sequencing artifacts. Examples of variant calling tools include MuTect, Strelka, and JointSNVMix2, which are capable of detecting structural alterations, copy number variations, and microsatellite instability in addition to smaller mutations. Detected variants, along with their properties (e.g., type, frequency, and genomic location), are annotated and stored in a variant call format (VCF) file. The number of variants detected per sample can range from approximately 1,500 to over 1 million, depending on the type and complexity of the sample.

### C. Training and Implementation of a Machine Learning Model

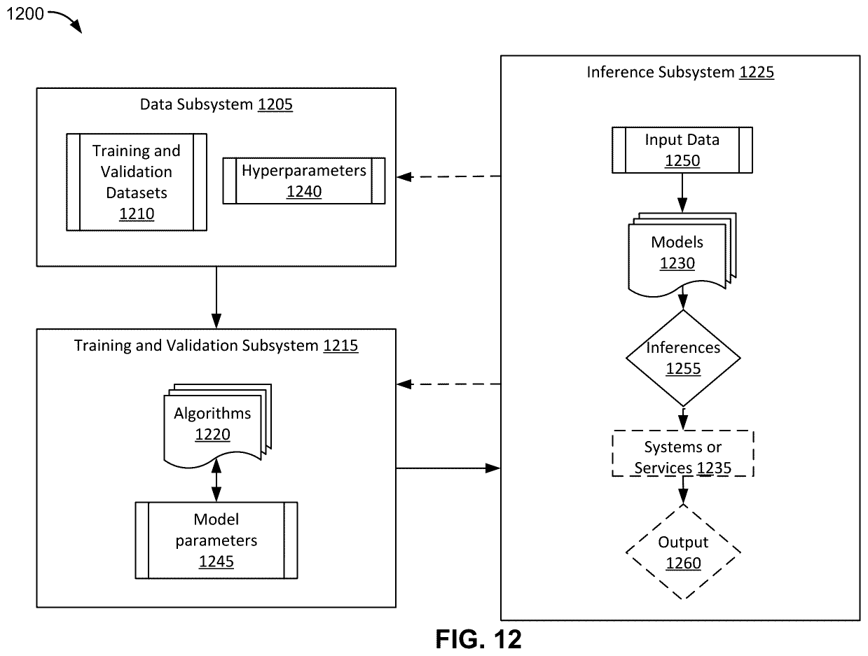

FIG. 12 shows a block diagram of an exemplary machine learning pipeline 1200 comprising several subsystems that work together to train, validate, and implement one or more machine learning models in accordance with various embodiments. The machine learning pipeline 1200 comprises a data subsystem 1205 for collecting, generating, preprocessing, and labeling of training and validation datasets 1210, and collecting, generating, setting, or implementing model hyperparameters 1240, a training and validation subsystem 1215 that facilitates the training and validation of one or more machine learning algorithms 1220 and the generation of one or more machine learning models 1230, and an inference subsystem 1225 for deploying and implementing the one or more trained machine learning models 1230 independently or in combination with one or more downstream applications 1235 for further processes (e.g., providing diagnosis or administering a treatment).

As used herein, machine learning algorithms (also described herein as simply algorithm or algorithms) are procedures that are run on datasets (e.g., training and validation datasets) and extract features from the datasets, perform pattern recognition on the datasets, learn from the datasets, and/or are fit on the datasets. Examples of machine learning algorithms include linear and logistic regressions, decision trees, random forest, support vector machines, principal component analysis, Apriori algorithms, gradient descent algorithms, Hidden Markov Model, artificial neural networks, k-means clustering, and k-nearest neighbors. As used herein, machine learning models (also described herein as simply model or models) are the output of the machine learning algorithms and are comprised of model parameters and prediction algorithm(s). In other words, the machine learning model is the program that is saved after running a machine learning algorithm on training data and represents the rules, numbers, and any other algorithm-specific data structures required to make inferences. For example, a linear regression algorithm may result in a model comprised of a vector of coefficients with specific values, a decision tree algorithm may result in a model comprised of a tree of if-then statements with specific values, a random forest algorithm may result in a random forest model that is an ensemble of decision trees for classification or regression, or neural network, backpropagation, and gradient descent algorithms together result in a model comprised of a graph structure with vectors or matrices of weights with specific values.

### 1. Data Subsystem

Data subsystem 1205 is used to collect, generate, preprocess, and label data to be used by the training and validation subsystem 1215 to train and validate one or more machine learning algorithms 1220. The data subsystem 1205 comprises training and validation datasets 1210 and model hyperparameters 1240. Raw data may be acquired through a public database or a commercial database. For example, the data subsystem 1205 may access and load data from a data storage structure such as a database, a laboratory or hospital information system, a clinical laboratory, or the like associated with any modality for acquiring health data for subjects. The data may include sequencing read data generated from NGS assays (e.g., WGS, WES, targeted sequencing, and the like). In some instances, the data is WGS data produced from a biological sample comprising tissue, cells, plasma, blood, cell free DNA, or circulating tumor DNA.

Preprocessing may be implemented by the data subsystem 1205, serving as a bridge between raw data acquisition and effective model training. The primary objective of preprocessing is to transform raw data into a format that is more suitable and efficient for analysis, ensuring that the data fed into machine learning algorithms is clean, consistent, and relevant. This step can be useful because raw data often comes with a variety of issues such as missing values, noise, irrelevant information, and inconsistencies that can significantly hinder the performance of a model. By standardizing and cleaning the data beforehand, preprocessing helps in enhancing the accuracy and efficiency of the subsequent analysis, making the data more representative of the underlying problem the model aims to solve.

Raw data preprocessing may comprise data synthesis and/or data augmentation. Different data synthesis and/or data augmentation techniques may be implemented by the data subsystem 1205 to generate pre-processed data to be used for the training and validation subsystem 1215. Data synthesizing involves creating entirely new data points from scratch. This technique may be used when real data is insufficient, too sensitive to use, or when the cost and logistical barriers to obtaining more real data are too high. The synthesized data should be realistic enough to effectively train a machine learning model, but distinct enough to comply with regulations (e.g., privacy regulations (such as the Health Insurance Portability and Accountability Act in the United States) and ethical guidelines), if necessary. Techniques such as Generative Adversarial Networks (GANs) or Variational Autoencoders (VAEs) may be used to generate new data examples. These models learn the distribution of real data and attempt to produce new data examples that are statistically similar but not identical. Data augmentation, on the other hand, refers to techniques used to artificially expand the size of a dataset by creating modified versions of existing data examples. The primary goal of data augmentation is to increase variation in the data in order to make the model more robust to variations it might encounter in the real world, thereby improving its ability to generalize from the training data to unseen data.

Other raw data preprocessing techniques include data cleaning, normalization, feature extraction, dimensionality reduction, and the like. Data cleaning may involve removing duplicates, filling in missing values, or filtering out outliers to improve data quality. Normalization involves scaling numeric values to a common scale without distorting differences in the ranges of values, which helps prevent biases in the model due to the inherent scale of features. Feature extraction involves transforming the input data into a set of useable features, possibly reducing the dimensionality of the data in the process. For instance, raw sequencing data might comprise the initial output generated by sequencing machines from a sequencing assay. This initial output is typically in the form of raw sequence reads, which are short nucleotide sequences (e.g., DNA or RNA) that represent fragments of the genome or transcriptome being sequenced. Feature extraction may transform the raw sequencing data into a set of features including coverage, mutant allele fraction, quality scores, and/or confidence scores. For example, a WGS and analysis assay produce a variety of different sequencing, alignment, mapping, variant calling, quality control files, and the like that each include all types of features that describe characteristics or properties of the sequencing, alignment/mapping, variant calling, and quality control files. Sequencing features extracted may include metrics from FASTQ files such as quality scores for any given base in the sequence data, quality of alignment, quality of reads, and metrics relating to the complexity of the region in the genome (e.g., repeat regions and other regions prone to NGS sequencing error). Variant calling features may also be extracted, including a confidence or probability score that is output by the variant caller when a variant is identified and/or the quality of the base of the variant. The number of features depends on the project's need, for example, about 10 features to about 500 features may be extracted.

Dimensionality reduction techniques like Principal Component Analysis (PCA) may be used to reduce the number of variables under consideration, by obtaining a set of principal variables. These techniques not only help in reducing the computational load on the model but also in mitigating issues like overfitting by simplifying the data without losing critical information.

In the instance that machine learning pipeline 1200 is used for supervised or semi-supervised learning of machine learning models, labeling techniques can be implemented as part of the data preprocessing. The quality and accuracy of data labeling directly influence the model's performance, as labels serve as the definitive guide that the model uses to learn the relationships between the input features and the desired output. Particularly in complex domains such as cancer detection and medical diagnosis, precise and consistent labeling is important because it provides the ground truth or target outcomes against which the model's predictions are compared and adjusted during training. Effective labeling ensures that the model is trained on correct and clear examples, thus enhancing its ability to generalize from the training data to real-world scenarios. In some instances, the ground truth value is provided within the raw data.

In some instances, the ground truth values (labels) are provided within the raw data. For example, when the raw data includes sequencing data, the labels may include variant types. Many different variant types may be included in the variant files accessed and loaded by the data subsystem 1205. For example, the variants may include benign, likely benign, variant of unknown significance, likely pathogenic or pathogenic variants. The variants may comprise germline variants, somatic variants, or a combination thereof. Different structural variants may be included such as small structural variants (less than 50 base pairs) such as single nucleotide variants (SNVs), single nucleotide polymorphisms (SNPs) and small structural sequence variants (SVs) (e.g., deletions, insertions, insertions and deletions, sometimes referred to as indels) and larger (e.g., greater than 50 base pairs) SVs such as chromosomal rearrangements (e.g., translocations and inversions). In some instances, the variant types may be substitutions, small indels, and larger alterations such as rearrangements, copy number variation, and microsatellite instabilities.

Labeling techniques can vary significantly depending on the type of data and the specific requirements of the project. Manual labeling, where human annotators label the data, is one method that can be used. This approach may be useful when a detailed understanding and judgment are required, such as in labeling medical data or categorizing text data where context and subtlety are important. However, manual labeling can be time-consuming and prone to inconsistency, especially with a large number of annotators. To mitigate this, semi-automated labeling tools may be used as part of data subsystem 1205 to pre-label data using algorithms, which human annotators may then review and correct as needed. Another approach is active learning, a technique where the model being developed is used to label new data iteratively. The model suggests labels for new data points, and human annotators may review and adjust certain predictions such as the most uncertain predictions. This technique optimizes the labeling effort by focusing human resources on a subset of the data, e.g., the most ambiguous cases, improving efficiency and label quality through continuous refinement.

For example, when the raw data includes sequencing data, the labels may include whether a variant is a true positive mutation or a false positive mutation. True positive mutations/variants can be obtained from clinical FFPE tissues, cell lines, plasma cases from patients with cancer or patients with a recurrence after a cancer treatment, or any combination thereof. False positive mutations/variants can be obtained from noncancerous normal FFPE tissues, cells, plasma cases from noncancerous samples or patients without a recurrence after a cancer treatment, or any combination thereof. When a variant is partial-labeled or left unlabeled, a user may update the label of the variant or make an annotation to indicate what portion of the input data should be labeled.

The training and validation datasets 1210 may comprise the raw data and/or the preprocessed data. The training and validation datasets 1210 are typically split into at least three subsets of data: training, validation, and testing. The training subset is used to fit the model, where the model is configured to make inferences based on the training data. The validation subset, on the other hand, is utilized to tune hyperparameters and prevent overfitting to the training data. Finally, the testing subset serves as a new and unseen dataset for the model, used to simulate real-world applications and evaluate the final model's performance. The process of splitting ensures that the model can perform well not just on the data it was trained on, but also on new, unseen data, thereby validating and testing its ability to generalize.

Various techniques can be employed to split the data effectively, aiming to maintain a good representation of the overall dataset in each subset. A simple random split (e.g., a 70/20/10%, 80/10/10%, or 60/25/15%) is the most straightforward approach, where examples from the data are randomly assigned to each of the three sets. However, more sophisticated techniques may be necessary to preserve the underlying distribution of data. For instance, stratified sampling may be used to ensure that each split reflects the overall distribution of a specific variable, particularly useful in cases where certain categories or outcomes are underrepresented. Another technique, k-fold cross-validation, involves rotating the validation set across different subsets of the data, maximizing the use of available data for training while still holding out portions for validation. These techniques help in achieving more robust and reliable model evaluation and are useful in the development of predictive models that perform consistently across datasets.

Data subsystem 1205 can also be used for collecting, generating, setting, or implementing model hyperparameters 1240 for the training and validation subsystem 1215. The hyperparameters control the overall behavior of the models. Unlike model parameters 1245 that are learned automatically during training, model hyperparameters 1240 are settings that are external to the model and must be determined before training begins. Model hyperparameters 1240 can have a significant impact on the performance of the model. For example, in a neural network, model hyperparameters 1240 include the learning rate, number of layers, number of neurons per layer, and/or activation functions, among others; in a random forest, model hyperparameters 1240 may include the number of decision trees in the forest, the maximum depth of each decision tree, the minimum number of samples required to be at each leaf node, the maximum number of features to consider when looking for a best split, and/or bootstrap parameters. These settings can determine how quickly a model learns, its capacity to generalize from training data to unseen data, and its overall complexity. Correctly setting hyperparameters is important because inappropriate values can lead to models that underfit or overfit the data. Underfitting occurs when a model is too simple to learn the underlying pattern of the data, and overfitting happens when a model is too complex, learning the noise in the training data as if it were signal. Many different variant types may be included in the variant files accessed and loaded by data generator 1205. For example, the variants may include benign, likely benign, variant of unknown significance, likely pathogenic or pathogenic variants. The variants may comprise germline variants, somatic variants, or a combination thereof. Different structural variants may be included such as small structural variants (less than 50 base pairs) such as single nucleotide variants (SNVs), single nucleotide polymorphisms (SNPs) and small structural sequence variants (SVs) (e.g., deletions, insertions, insertions and deletions, sometimes referred to as indels) and larger (greater than 50 base pairs) SVs such as chromosomal rearrangements (e.g., translocations and inversions). In some embodiments the variants may be substitutions, small indels, and larger alterations such as rearrangements, copy number variation, and microsatellite instabilities.

### 2. Training and Validation Subsystem

The training and validation subsystem 1215 is comprised of a combination of specialized hardware and software to efficiently handle the computational demands required for training, validating, and testing machine learning algorithm/model. On the hardware side, high-performance GPUs (Graphics Processing Units) may be used for their ability to perform parallel processing, drastically speeding up the training of complex models, especially deep learning networks. CPUs (Central Processing Units), while generally slower for this task, may also be used for less complex model training or when parallel processing is less critical. TPUs (Tensor Processing Units), designed specifically for tensor calculations, provide another level of optimization for machine learning tasks. In some instances, a Field-Programmable Gate Array (FPGA), or a specifically designed FPGA may be used to perform the training, validating, and/or testing tasks,

Training is the initial phase of developing machine learning models 1230 where the model learns to make predictions, classifications, or decisions based on training data provided from the training and validation datasets 1210. During this phase, the model iteratively adjusts its internal model parameters 1245 to achieve a preset optimization condition. In a supervised machine learning training process, the preset optimization condition can be achieved by minimizing the difference between the model output (e.g., predictions, classifications, or decisions) and the ground truth labels in the training data. In some instances, the preset optimization condition can be achieved when the preset fixed number of iterations or epochs (full passes through the training dataset) is reached. In some instances, the preset optimization condition is achieved when the performance on the validation dataset stops improving or starts to degrade. In some instances, the preset optimization condition is achieved when a convergence criterion is met, such as when the change in the model parameters falls below a certain threshold between iterations. This process, known as fitting, is fundamental because it directly influences the accuracy and effectiveness of the model.

In an exemplary training phase performed by the training and validation subsystem 1215, the training subset of data is input into the machine learning algorithms 1220 to find a set of model parameters 1245 (e.g., weights, coefficients, trees, feature importance, and/or biases) that minimizes or maximizes an objective function (e.g., a loss function, a cost function, a contrastive loss function, a cross-entropy loss function, an Out-of-Bag (OOB) score, etc.). To train the machine learning algorithms 1220 to achieve accurate predictions, “errors” (e.g., a difference between a predicted label and the ground truth label) need to be minimized. In order to minimize the errors, the model parameters can be configured to be incrementally updated by minimizing the objective function over the training phase (“optimization”). Various techniques may be used to perform the optimization. For example, to train machine learning algorithms such as a neural network, optimization can be done using back propagation. The current error is typically propagated backwards to a previous layer, where it is used to modify the weights and bias in such a way that the error is minimized. The weights are modified using the optimization function. Other techniques such as random feedback, Direct Feedback Alignment (DFA), Indirect Feedback Alignment (IFA), Hebbian learning, and the like can also be used to update the model parameters 445 in a manner as to minimize or maximize an objective function. This cycle is repeated until a desired state (e.g., a predetermined minimum value of the objective function) is reached.

The training phase is driven by three primary components: the model architecture (which defines the structure of the algorithm(s) 1220), the training data (which provides the examples from which to learn), and the learning algorithm (which dictates how the model adjusts its model parameters). The goal is for the model to capture the underlying patterns of the data without memorizing specific examples, thus enabling it to perform well on new, unseen data.

The model architecture is the specific arrangement and structure of the various components and/or layers that make up a model. In the context of a neural network, the model architecture may include the configuration of layers in the neural network, such as the number of layers, the type of layers (e.g., convolutional, recurrent, fully connected), the number of neurons in each layer, and the connections between these layers. In the context of a random forest consisting of a collection of decision trees, the model architecture may include the configuration of features used by the decision trees, the voting scheme, and hyperparameters such as the number of trees in the forest, the maximum depth of each tree, the minimum number of samples required to split a node, and the maximum number of features to consider when looking for the best split. In some instances, the model architecture is configured to perform multiple tasks. For example, a first component of the model architecture may be configured to perform a feature selection function, and a second component of the model architecture may be configured to perform a feature scoring function. The different components may correspond to different algorithms or models, and the model architecture may be an ensemble of multiple components.

Model architecture also encompasses the choice and arrangement of features and algorithms used in various models, such as decision trees or linear regression. The architecture determines how input data is processed and transformed through various computational steps to produce the output. The model architecture directly influences the model's ability to learn from the data effectively and efficiently, and it impacts how well the model performs tasks such as classification, regression, or prediction, adapting to the specific complexities and nuances of the data it is designed to handle.

The model architecture can encompass a wide range of algorithms 1220, suitable for different kinds of tasks and data types. Examples of algorithms 1220 include, without limitation, linear regression, logistic regression, decision tree, Support Vector Machines, Naives Bayes algorithm, Bayesian classifier, linear classifier, K-Nearest Neighbors, K-Means, random forest, dimensionality reduction algorithms, grid search algorithm, genetic algorithm, AdaBoosting algorithm, Gradient Boosting Machines, and Artificial Neural Networks such as convolutional neural network (“CNN”), an inception neural network, a U-Net, a V-Net, a residual neural network (“Resnet”), a transform neural network, a recurrent neural network, a Generative adversarial network (GAN), or other variants of Deep Neural Networks (“DNN”) (e.g., a multi-label n-binary DNN classifier or multi-class DNN classifier). These algorithms can be implemented using various machine learning libraries and frameworks such as TensorFlow, PyTorch, Keras, and scikit-learn, which provide extensive tools and features to facilitate model building, training, validation, and testing.

The learning algorithm is the overall method or procedure used to adjust the model parameters 1245 to fit the data. It dictates how the model learns from the data provided during training. This includes the steps or rules that the algorithm follows to process input data and adjust the model's internal parameters (e.g., weights in neural networks) based on the output of the objective function. Examples of learning algorithms include gradient descent, backpropagation for neural networks, and splitting criteria in decision trees.

Various techniques may be employed by training and validation subsystem 1215 to train machine learning models 1230 using the learning algorithm, depending on the type of model and the specific task. For supervised learning models, where the training data includes both inputs and expected outputs (e.g., ground truth labels), gradient descent is a possible method. This technique iteratively adjusts the model parameters 1245 to minimize or maximize an objective function (e.g., a loss function, a cost function, a contrastive loss function, etc.). The objective function is a method to measure how well the model's predictions match the actual labels or outcomes in the training data. It quantifies the error between predicted values and true values and presents this error as a single real number. The goal of training is to minimize this error, indicating that the model's predictions are, on average, close to the true data. Common examples of loss functions include mean squared error for regression tasks and cross-entropy loss for classification tasks.

The adjustment of the model parameters 1245 is performed by the optimization function or algorithm, which refers to the specific method used to minimize (or maximize) the objective function. The optimization function is the engine behind the learning algorithm, guiding how the model parameters 1245 are adjusted during training. It determines the strategy to use when searching for the best weights that minimize (or maximize) the objective function. Gradient descent is a primary example of an optimization algorithm, including its variants like stochastic gradient descent (SGD), mini-batch gradient descent, and advanced versions like Adam or RMSprop, which provide different ways to adjust learning rates or take advantage of the momentum of changes. For example, in training a neural network, backpropagation may be used with gradient descent to update the weights of the network based on the error rate obtained in the previous epoch (cycle through the full training dataset). Another technique in supervised learning is the use of decision trees, where a tree-like model of decisions is built by splitting the training dataset into subsets based on an attribute value test. This process is repeated on each derived subset in a recursive manner called recursive partitioning. In training a random forest, the set of decision trees can be trained collectively to minimize a Gini impurity or entropy, leading to accurate classification.

In unsupervised learning, where training data does not include labels, different techniques are used. Clustering is one method where data is grouped into clusters that maximize the similarities of data within the same cluster and maximize the differences with data in other clusters. The K-Means algorithm, for example, assigns each data point to the nearest cluster by minimizing the sum of distances between data points and their respective cluster centroids. Another technique, Principal Component Analysis (PCA), involves reducing the dimensionality of data by transforming it into a new set of variables, the principal components, which are uncorrelated and ordered so that the first few retain most of the variation present in all of the original variables. These techniques help uncover hidden structures or patterns in the data, which can be essential for feature reduction, anomaly detection, or preparing data for further supervised learning tasks.

Validating is another phase of developing machine learning models 1230 where the model is checked for deficiencies in performance and the hyperparameters 1240 are optimized based on validation data provided from the training and validation datasets 1210. The validation data helps to evaluate the model's performance, such as accuracy, precision, or recall, to gauge how well the model is likely to perform in real-world scenarios. Hyperparameter optimization, on the other hand, involves adjusting the settings that govern the model's learning process (e.g., learning rate, number of layers, size of the layers in neural networks) to find the combination that yields the best performance on the validation data. One optimization technique is grid search, where a set of predefined hyperparameter values are systematically evaluated. The model is trained with each combination of these values, and the combination that produces the best performance on the validation set is chosen. Although thorough, grid search can be computationally expensive and impractical when the hyperparameter space is large. A more efficient alternative optimization technique is random search, which samples hyperparameter combinations from a defined distribution randomly. This approach can in some instances find a good combination of hyperparameter values faster than grid search. Advanced methods like Bayesian optimization, genetic algorithms, and gradient-based optimization may also be used to find optimal hyperparameters more effectively. These techniques model the hyperparameter space and use statistical methods to intelligently explore the space, seeking hyperparameters that yield improvements in model performance.

An exemplary validation process includes iterative operations of inputting the validation subset of data into the trained algorithm(s) using a validation technique such as K-Fold Cross-Validation, Leave-one-out Cross-Validation, Leave-one-group-out Cross-Validation, Nested Cross-Validation, or the like, to fine tune the hyperparameters and ultimately find the optimal set of hyperparameters. In some instances, a 5-fold cross-validation technique may be used to avoid overfitting the trained algorithm and/or to limit the number of selected features per split to the square-root of the total number of input features. In some instances, training dataset is split into 5 equal-size cohorts (or about equal-size), and every four of the cohorts are used to train an algorithm to generate five models (e.g, cohorts #1, 2, 3, and 4 are used to train and generate model 1, cohorts #1, 2, 3, and 5 are used to train and generate model 2, cohorts #1, 2, 4, and 5 are used to train and generate model 3, cohorts #1, 3, 4, and 5 are used to train and generate model 4, and cohorts #2, 3, 4 and 5 are used to train and generate model 5). Each model is evaluated (or validated) using the unused cohort in the training (e.g., for model 5, cohort #1 is used for validation). The overall performance of the training can be evaluated by an average performance of the five models. K-fold cross-validation provides a more robust estimate of a model's performance compared to a single training/validation split because it utilizes the entire dataset for both training and evaluation and reduces the variance in the performance estimate.

Once a machine learning model has been trained and validated, it undergoes a final evaluation using testing data provided from the training and validation datasets 1210, which is a separate subset of the training and validation datasets 1210 that generally has not been used during the training or validation phases. This step is crucial as it provides an unbiased assessment of the model's performance in simulating real-world operation. The test dataset serves as new, unseen data for the model, mimicking how the model would perform when deployed in actual use. During testing, the model's predictions are compared against the true values in the test dataset using various performance metrics such as accuracy, precision, recall, and mean squared error, depending on the nature of the problem (classification or regression). This process helps to verify the generalizability of the model-its ability to perform well across different data samples and environments-highlighting potential issues like overfitting or underfitting and ensuring that the model is robust and reliable for practical applications. The machine learning models 1230 are fully validated and tested once the output predictions have been deemed acceptable by user defined acceptance parameters. Acceptance parameters may be determined using correlation techniques such as Bland-Altman method and the Spearman's rank correlation coefficients and calculating performance metrics such as the error, accuracy, precision, recall, receiver operating characteristic curve (ROC), and the like.

### 3. Inference Subsystem

The inference subsystem 1225 is comprised of various components for deploying the machine learning models 1230 in a production environment. Deploying the machine learning models 1230 includes moving the models from a development environment (e.g., the training and validation subsystem 1215, where it has been trained, validated, and tested), into a production environment where it can make inferences on real-world data (e.g., input data 1250). This step typically starts with the model being saved after training, including its parameters and configuration such as final architecture and hyperparameters.

Once deployed, the model is ready to receive input data 1250 and return outputs (e.g., inferences 1255). In some instances, the model resides as a component of a larger system or service (e.g., including additional downstream applications 1235). In some instances, the models 1230 and/or the inferences 1255 can be used by the downstream applications 1235 to provide further information. For example, the inferences 1255 can be used to determine whether a specific treatment should be administered to a patient. The downstream applications can be configured to generate an output 1260. In some instances, the output 1260 comprises a report including inferences 1255 and information generated by the downstream applications 1235.

In an exemplary inference subsystem 1225, the input data 1250 includes sequencing and variant files generated from one or more biological samples from a patient having been diagnosed a disease (e.g., cancer). The input data 1250 may further include clinical data for the same patient that provides information on the type/stage of disease, past, current, and/or future treatment plans, whether the patient has had a recurrence of the disease, and any other information pertinent to the patient. In some instances, the input data 1250 comprises clinicopathological risk factors that are associated with distinction of patients whether they are at either a very low risk or a very high-risk of developing a recurrence of the cancer within a certain amount of time (e.g., 3 years). The sequencing and variant files may be generated by performing WGS and variant calling on the biological sample (e.g., plasma) collected from the patient by the example workflow 1100 as described with respect to FIG. 11.

In some instances, the input data 1250 may be preprocessed before inputting into the models 1230 to achieve a faster model performance. For example, the input data 1250 may be preprocessed by the workflow 1100 as described with respect to FIG. 11. The preprocessing may reduce the dimensions of the input data 1250 and thus save computing time and resources (e.g., requiring less computer memory) in the inference stage to generate the inferences 1255.

To manage and maintain its performance, a deployed model may also be continuously monitored to ensure it performs as expected over time. This involves tracking the model's prediction accuracy, response times, and other operational metrics. Additionally, the model may require retraining or updates based on new data or changing conditions. This can be useful because machine learning models can drift over time due to changes in the underlying data they are making predictions on—a phenomenon known as model drift. Therefore, maintaining a machine learning model in a production environment often involves setting up mechanisms for performance monitoring, regular evaluations against new test data, and potentially periodic updates and retraining of the model to ensure it remains effective and accurate in making predictions.

### IV. Examples

References and citations to other documents, such as patents, patent applications, patent publications, journals, books, papers, web contents, have been made throughout this disclosure. All such documents are hereby incorporated herein by reference in their entirety for all purposes.

Various modifications of the disclosure and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of techniques disclosed herein in its various embodiments and equivalents thereof.

The use of WES allows for a targeted analysis of protein-coding regions, reducing sequencing costs and data complexity while focusing on regions most likely to harbor disease-causing mutations. At the same time, the ability to process data on local or cloud-based servers provides scalability and flexibility, enabling efficient analysis across multiple samples. By generating comprehensive somatic mutation reports, this approach supports precision oncology applications, allowing oncologists to tailor treatments based on the genetic profile of a patient's tumor. Together, these advancements represent a significant step forward in clinical genomics, addressing the limitations of conventional NGS workflows and enhancing the ability to deliver personalized cancer care. These techniques unlock the full capabilities of NGS in the field of genomic technology, particularly for somatic mutation detection and classification, the design of precision medicine strategies, and the development of personalized treatment plans tailored to individual patients.

### A. Examples Related to Whole Exome Sequencing

The disclosure outlines a method for somatic mutation identification that leverages machine learning strategies to enhance the sensitivity and specificity of detecting true genetic alterations. This approach was compared to existing methods for somatic mutation identification using simulated datasets and experimentally validated whole-exome and targeted sequencing analyses to assess its overall accuracy. The study evaluated the concordance of this machine learning-based method with mutation calls from The Cancer Genome Atlas (TCGA) exomes and investigated the underlying causes of erroneous calls, including those occurring in actionable driver genes. Additionally, the impact of discordant mutation calls on tumor mutational burden (TMB) and clinical responses to cancer immunotherapy was assessed.

To determine the clinical significance of high-quality mutation analysis, the study conducted head-to-head comparisons of clinical sequencing workflows with and without the integration of machine learning methods. These comparisons emphasized the critical role of machine learning in improving the accuracy and reliability of mutation detection for both research and clinical applications. Ultimately, these analyses underscore the necessity of robust somatic mutation detection for interpreting large-scale genomic studies and for implementing these advancements in clinical practice, particularly in precision oncology and personalized medicine.

### A Machine Learning Approach for Somatic Mutation Discovery.

A method for analyzing next-generation cancer sequence data called Cerebro that uses machine learning to identify high confidence somatic mutations while minimizing false positives.

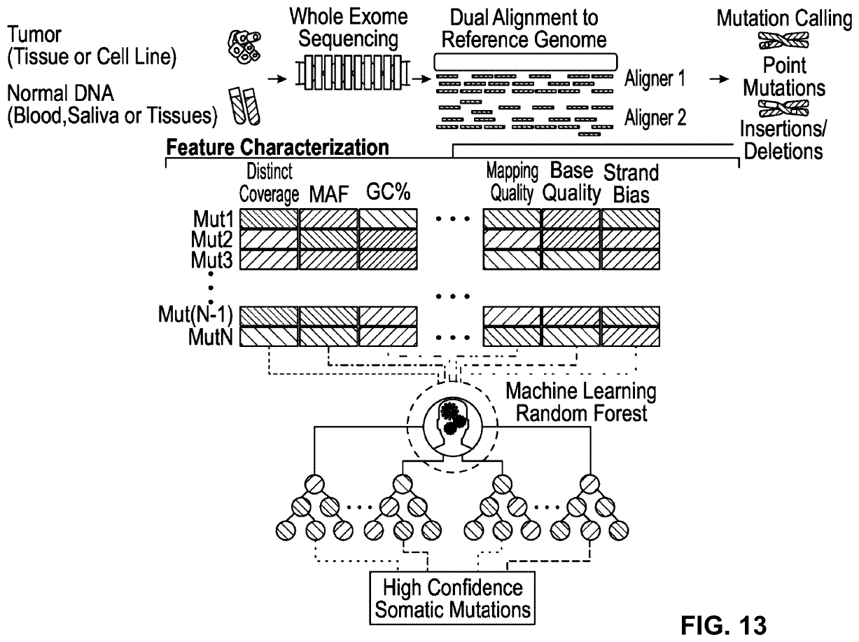

FIG. 13 is a diagram of a method of mutation detection using Cerebro. Cerebro utilizes a specialized random forest classification model that evaluates a large set of decision trees (e.g., at least 1,000) to generate a confidence score for each candidate variant. The model was trained using a normal peripheral blood DNA sample where exome regions were captured and sequenced twice using NGS methods. A total of 30,000 somatic variants comprising substitutions, insertions and deletions at mutant allele fractions from 1.5% to 100% were introduced in silico into one set of NGS data in order to provide the classifier with a training set of “tumor-specific” mutations as well as a representative source of NGS errors and artifacts that might otherwise be mislabeled as variants. The second NGS sequence data set from the same sample was used as the matched normal, and the combined data sets were analyzed for detection of somatic variants.

Over 300 features were considered that would optimize performance for identifying true somatic variants. Ultimately, 15 feature categories were selected from two separate alignment programs that included alignment characteristics (mapping quality, mismatches), sequence quality information (coverage, base quality) and information related to specific alterations (allele frequency, nearby sequence complexity, presence of alteration in matching normal specimen). Once implemented in certain embodiments, Cerebro utilized 1,000 decision trees for analysis of each mutation, with each tree evaluating a unique combination of the selected information supporting a candidate variant. The resultant confidence score from the Cerebro model represented the proportion of decision trees that would classify a candidate variant as somatic.

### Evaluation of Mutation Calling Accuracy

To systematically assess the accuracy of Cerebro for mutation detection, a series of validation studies using simulated and experimental cancer exomes were designed. The performance of this approach was evaluated by comparing it to existing software tools commonly used for somatic variant identification in research and clinical genomic analyses.

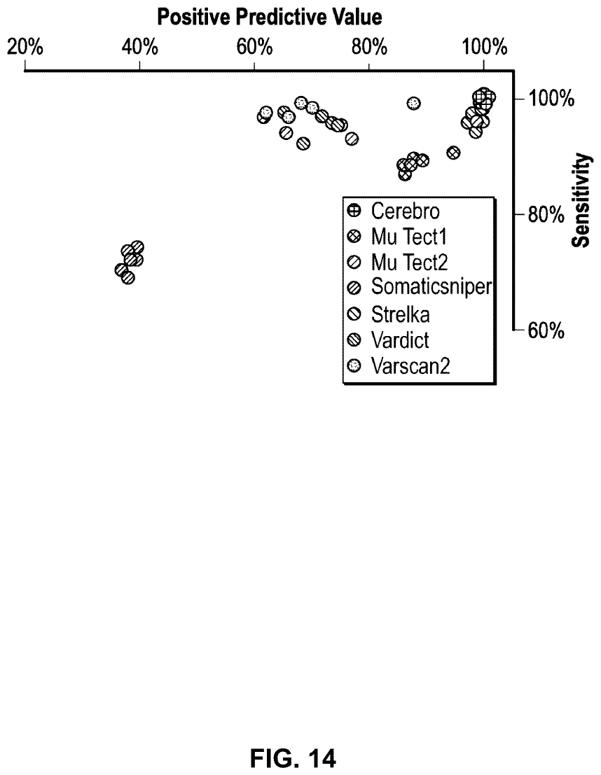

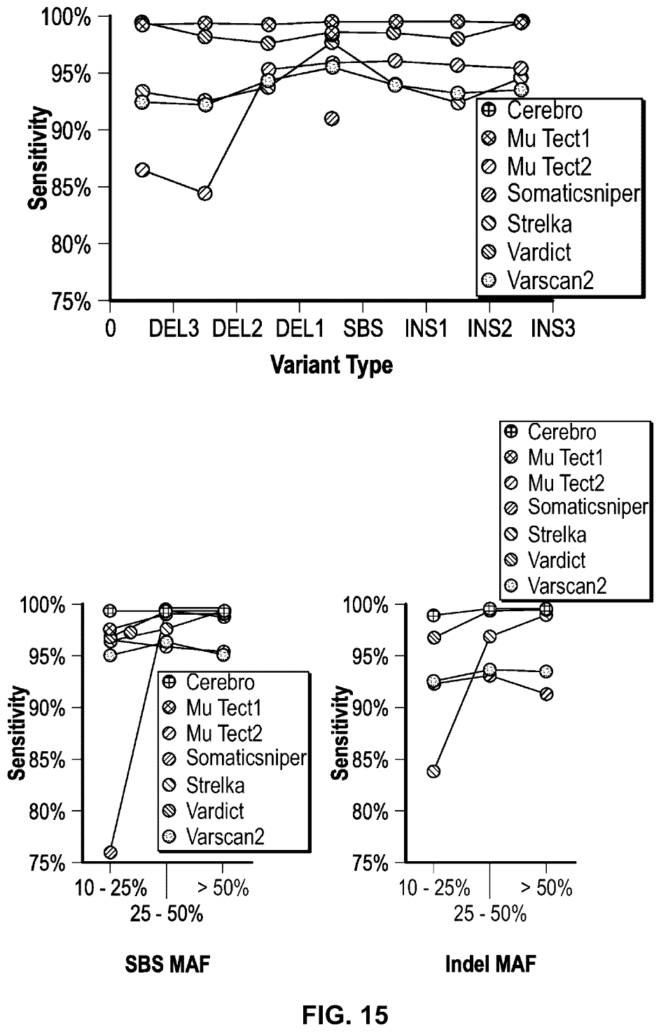

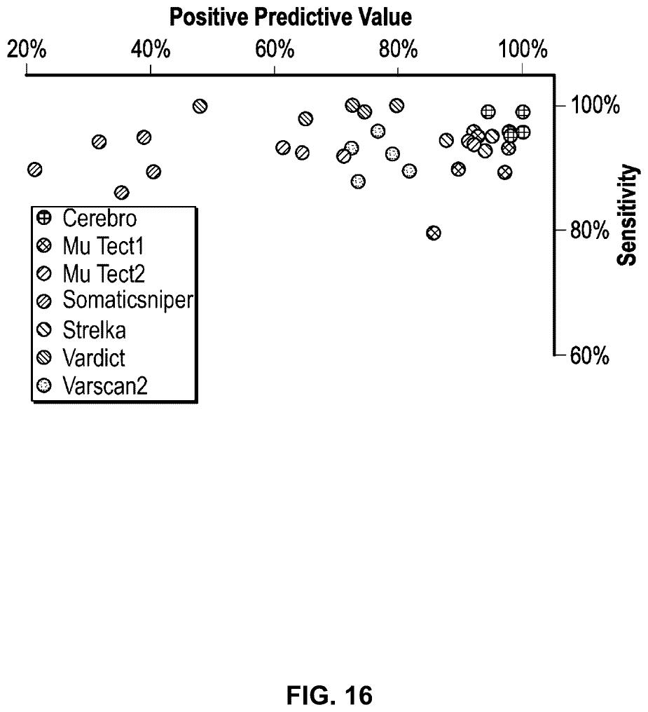

FIGS. 11-13 show validation study performance results. FIG. 14 shows positive predictive value vs. sensitivity for simulated low-purity tumor datasets created from normal cell line sequence data. FIG. 15 shows sensitivity stratified by mutation type, calculated from simulated mutations across mutation types and allele frequencies. FIG. 16 gives positive predictive value vs. sensitivity for cell line datasets with experimentally validated somatic mutations. DEL=deletion, INS-insertion; 1, 2, or 3=length of indel; SBS=single base substitution; MAF=mutant allele frequency. Three studies using a set of six normal cell lines with NGS exome data was performed. Two of those studies contained simulated mutations, with in silico somatic mutations spiked into a normal DNA sample. In the first study, low-purity tumors were simulated by incorporating 132 coding somatic mutations (120 SBS and 12 indels) with mutant allele frequencies ranging from 10% to 25% in the exome data (FIG. 14). To characterize false positive rates for the various tools, technical replicate exome pairs of the six normal cell lines were analyzed. Additionally, simulated exomes with in silico spike-ins of 7,000 somatic SBS and indel changes with variable mutant allele frequencies (ranging from 10% to 100%) were created, comprising a total of 42,000 somatic changes across the six samples that could be detected through these approaches (FIG. 16). This last study allowed for the examination of sensitivity of the various tools on specific mutation types and allele frequencies. In all cases, the location, type and level of in silico alterations were different from those utilized in the training of the Cerebro algorithm. Overall, substantial variation in sensitivity and positive predictive value among the tested variant classification programs (FIGS. 11 & 12) were observed. Cerebro maintained the highest level of sensitivity and positive predictive value, while other methods resulted in moderate to high false positive rates (FIG. 14). False positive calls by other methods were frequently associated with indicators such as poor tumor/normal coverage, low mutant base quality, and low mutant mapping quality.

To assess mutation detection performance using independently obtained experimental data, five matched tumor and normal specimens for which somatic mutations had been previously identified and validated through independent whole-exome sequencing were analyzed. These previous analyses carefully evaluated the entire coding sequences of the samples through PCR amplification of 173,000 coding regions and Sanger sequencing of the amplification products. Any observed alteration had been re-sequenced in the tumor and normal sample in order to confirm its tumor origin. Because Sanger sequencing analyses were designed to identify only clonal or near clonal alterations, Sanger validated alterations previously observed in this set of samples (n=314) were supplemented with additional bona fide changes that were identified by a consensus of multiple NGS mutation callers (n=163), or were detected by up to two mutation callers and validated using droplet digital PCR (n=18), a highly sensitive method for detection of alterations in a subset of DNA molecules. Comparison of all mutation callers to this reference set of alterations revealed that Cerebro had the highest overall accuracy compared to other methods (FIG. 16).

Evaluation of Tumor Exomes from TCGA

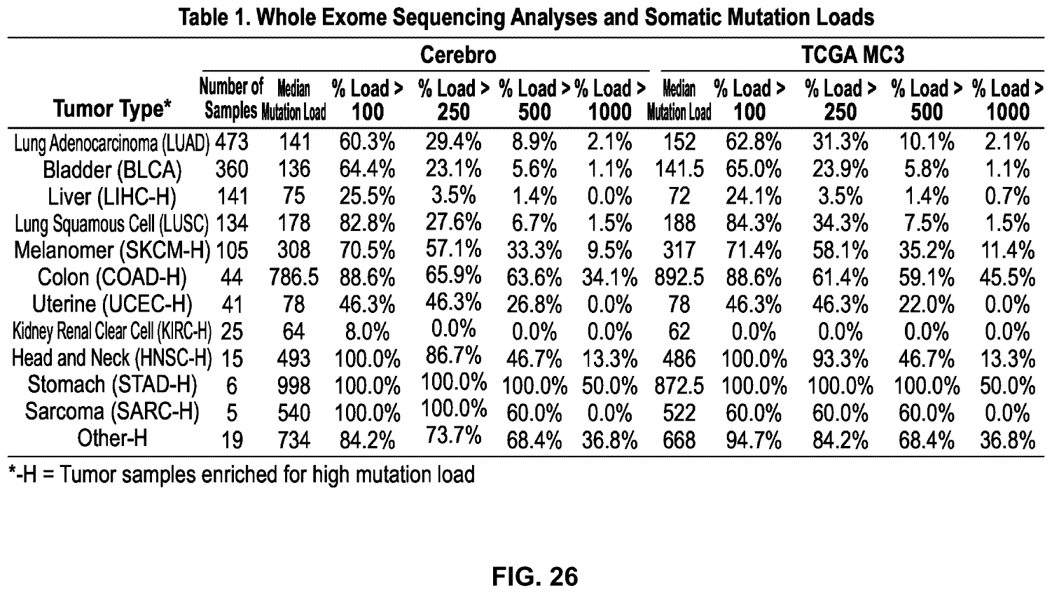

Whether the improved capabilities of Cerebro could be used to increase accuracy of mutation calling in large-scale cancer genome sequencing efforts including TCGA were assessed, as these serve as the basis for various research efforts in human cancer. Cerebro was used to analyze paired tumor-normal exomes from 1,368 patients in TCGA, focusing on tumors that would be relevant for both targeted therapies and immunotherapy. This set consisted of all available patients with non-small cell lung adenocarcinoma, non-small cell lung squamous cell carcinoma, and bladder urothelial carcinoma, as well as selected patients with higher mutational loads that had colorectal, gastric, head and neck, hepatocellular, renal, uterine cancer, or melanoma (Table 1 in FIG. 26). A total of 365,539 somatic alterations were identified, with an average of 267 somatic alterations per tumor (range, 1-5,871).

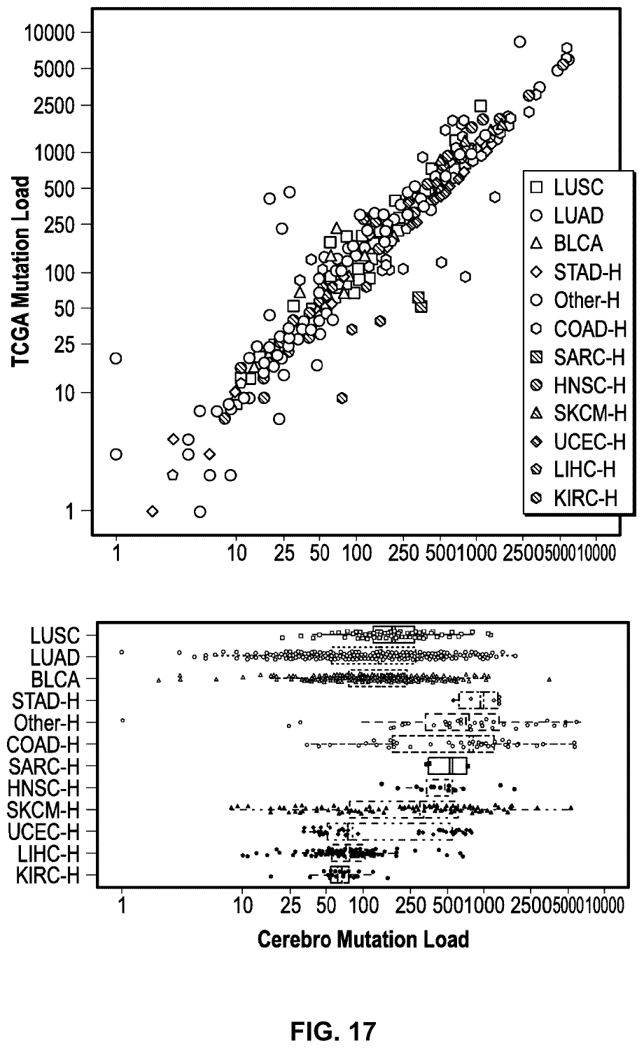

FIGS. 14 and 15 give a comparison of TCGA and Cerebro mutations for 1,368 exomes. Somatic mutations from the TCGA MC3 project and Cerebro were compared for concordance. FIG. 17 shows the total mutational loads between the two mutation calls shared by cancer type. LUSC=lung squamous cell carcinoma; LUAD=lung adenocarcinoma; BLCA=bladder; STAD=stomach; COAD-colorectal; SARC=sarcoma; HNSC=head and neck squamous cell; SKCM-melanoma; UCEC-uterine; LIHC=liver; KIRC=kidney; *−H=set enriched for high mutation load samples.

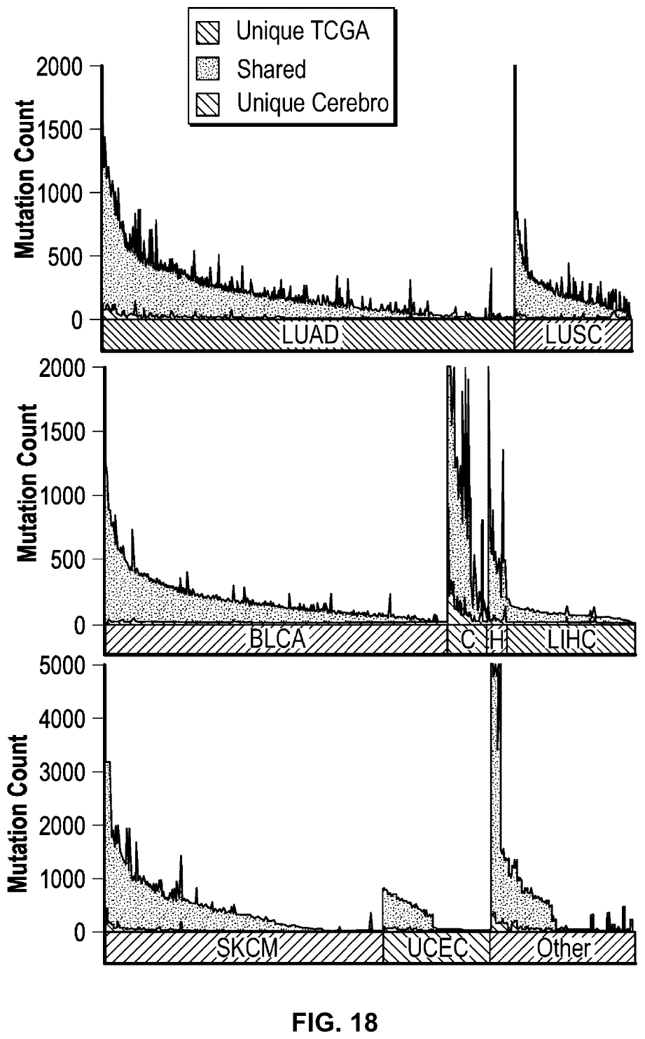

FIG. 18 reports unique/shared status for somatic mutations across all samples. C-colorectal; H=head and heck squamous cell. The total number of somatic mutations measured across the various tumor types was largely consistent with previous analyses of cancer exomes (FIG. 17). A significant positive correlation for mutation loads between Cerebro and the TCGA PanCanAtlas MC3 method that utilizes consensus calls among seven different mutation callers (Pearson correlation coefficient=0.93, P<0.0001; FIG. 17) were found, with 74.0% of somatic mutations shared between the two approaches. However, 10.3% of calls detected by Cerebro (n=44,439) were apparently missed by TCGA while 15.7% of alterations identified by TCGA (n=68,138) were not considered somatic alterations by Cerebro (FIG. 18). Individual tumors that were re-analyzed by Cerebro had mutation loads that differed by as many as 390 (95%) fewer or 729 (800%) more alterations compared to original calls. Comparison of detected alterations between Cerebro and other TCGA call sets (MC3, FIREHOSE, MuTect2) showed increasing concordance of Cerebro calls from MuTect2 (least concordant; average 60.2%) to MC3 (most concordant; average 75.8%). These observations are consistent with previous analyses of individual mutation callers and support the notion that the use of multiple approaches in MC3 is likely to reduce the false positive observations resulting from individual methods but may still have additional errors compared to Cerebro.

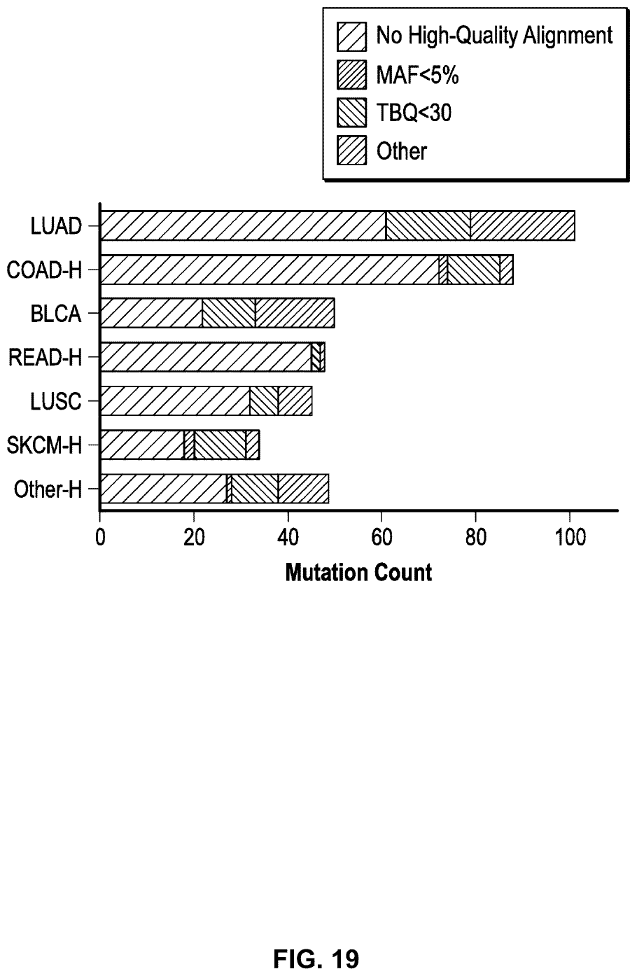

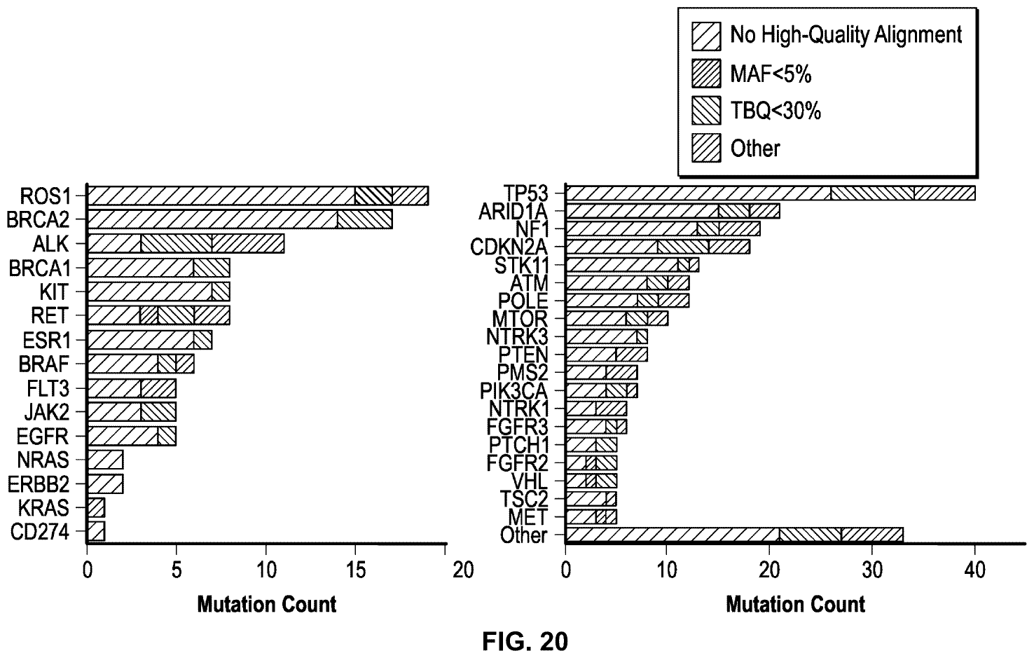

To more carefully evaluate discordant alterations in TCGA, somatic mutation calls for a set of 66 well-characterized cancer driver genes were investigated. FIGS. 16-18 give an analysis of cancer driver gene mutations. FIG. 19 shows that evaluation of mutations among 66 oncogenes and tumor suppressor genes indicated a large number of low confidence mutations unique to TCGA associated with various problematic features (No High-Quality Alignment=no consistent alignment found with at least 1 mutant base with quality higher than 30; MAF<5%=mutant allele frequency below 5%; TBQ<30-tumor base quality below 30). FIG. 20 is a distribution of problematic TCGA driver gene calls by genes with approved FDA therapies (left panel) or ongoing clinical trials (right panel).

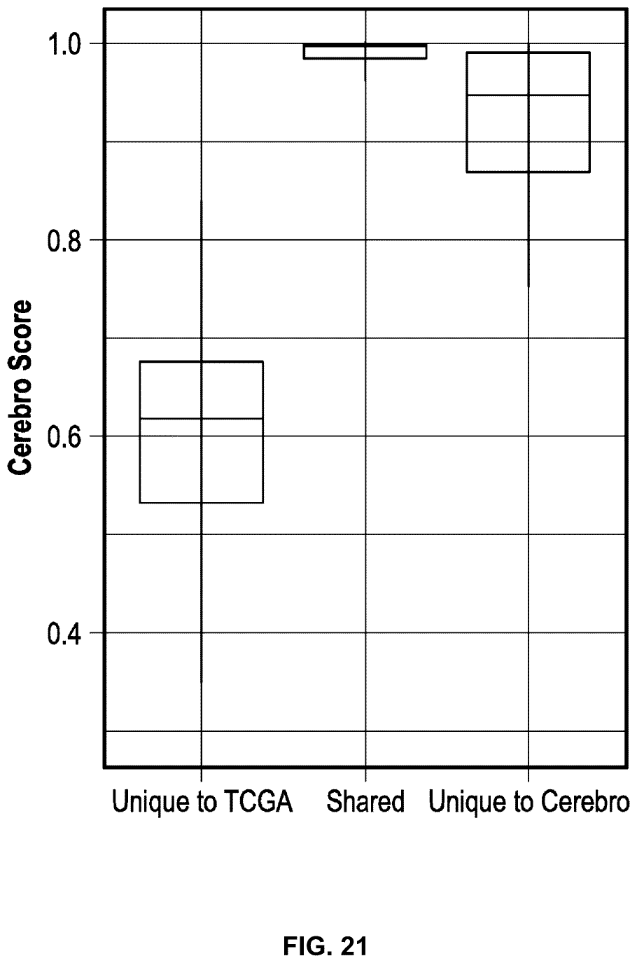

FIG. 21 gives the Cerebro confidence scores of mutations uniquely called by TCGA were significantly lower than other identified mutations. Of the 1,368 evaluated tumors, 1,257 (92%) had a mutation in this gene set, with 4,037 shared somatic mutations between TCGA and Cerebro, and a total of 777 alterations that were discordant between the analyses. Further examination revealed that most of the 429 mutations called only by TCGA were associated with poor sequence quality and alignment issues that were likely to not represent bona fide alterations (FIG. 19). Among driver genes associated with FDA-approved therapies or ongoing clinical trials (FIG. 20), low confidence TCGA mutations in 211 (16.8%) of patients was found. Quality confidence scores as determined by Cerebro of mutations uniquely called by TCGA were significantly lower than mutations observed by both platforms (P<0.0001; FIG. 21) or those that were uniquely detected by Cerebro (P<0.0001; FIG. 21). Overall, these results suggest that current TCGA data sets contain a significant number of false negative and positive somatic alterations in both passenger and driver genes among many tumor types, and the new mutation calls determined here provide an improved resource for analysis of TCGA sequence data.

### Somatic Mutation Burden and Response to Cancer Immunotherapy

To evaluate recently reported associations between total somatic mutation count (i.e., mutational burden) and response to immune checkpoint blockade, paired tumor-normal exome data from two recent studies including a study of response to anti-PD-1 therapy for 34 non-small cell lung cancer (NSCLC) patients, and a study of response to anti-CTLA-4 in 64 melanoma patients were obtained. Using Cerebro to analyze these NGS data, Cerebro results were compared to the mutations reported in the original publications, limiting the analyses to only nonsynonymous SBS changes as other types of alterations were not included in the published analyses. Across the NSCLC cohort, 9,049 and 6,385 mutations were identified in the original study and the re-analysis, respectively. In the melanoma cohort, 25,753 and 32,092 mutations were identified by the original publication and the re-analysis, respectively.

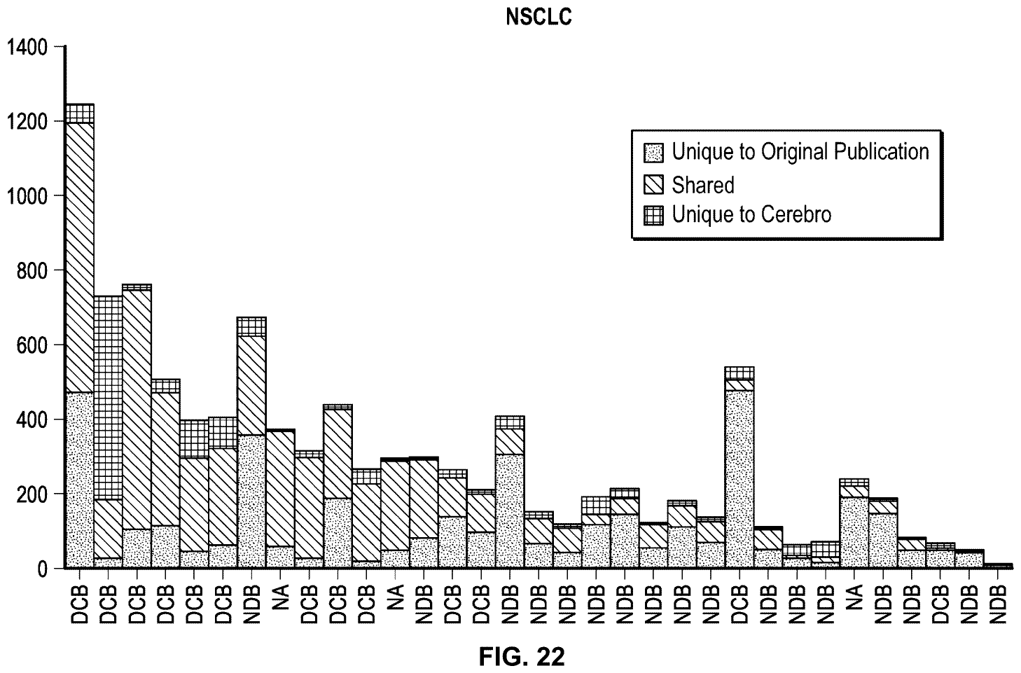

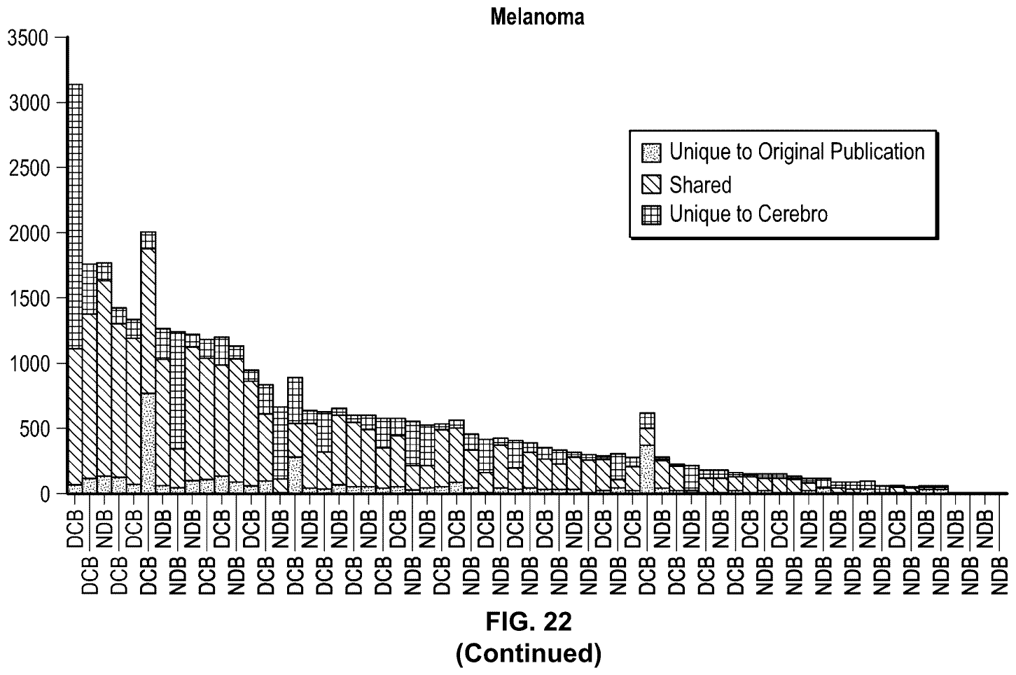

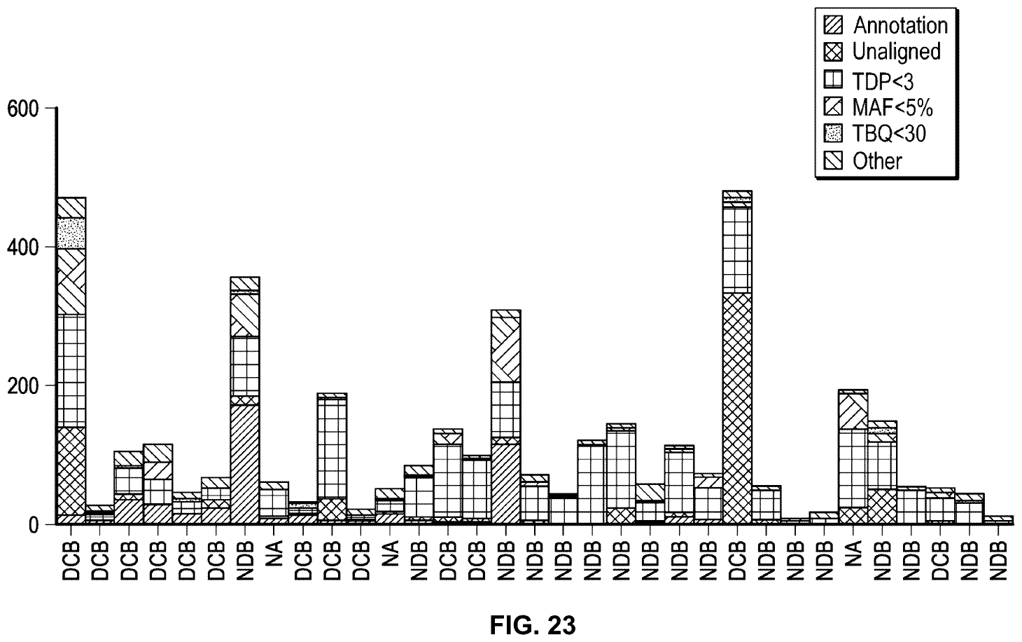

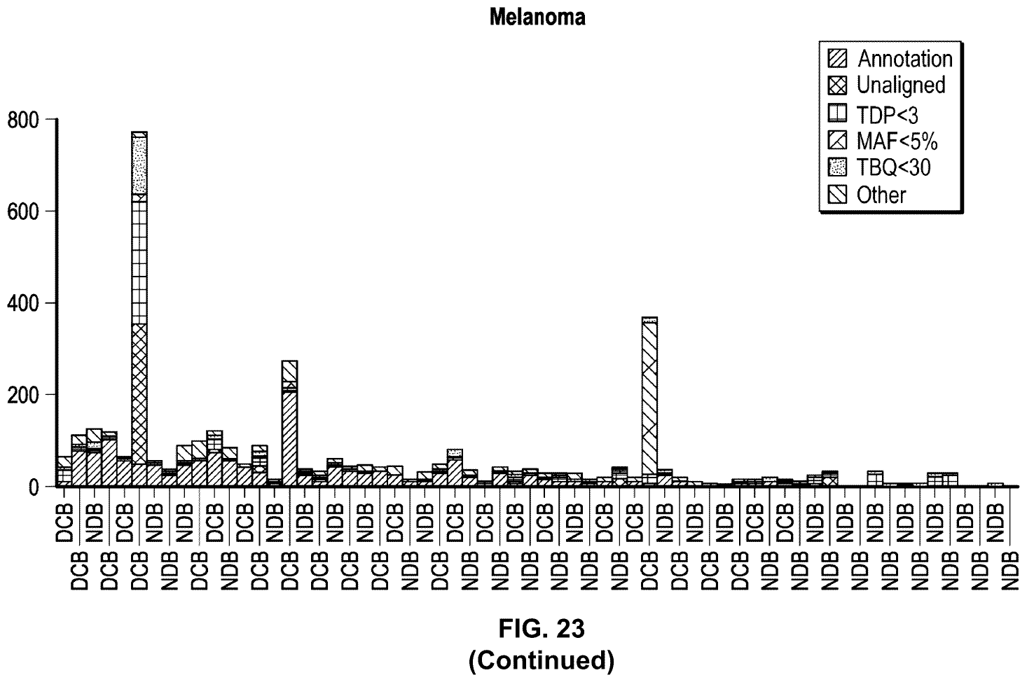

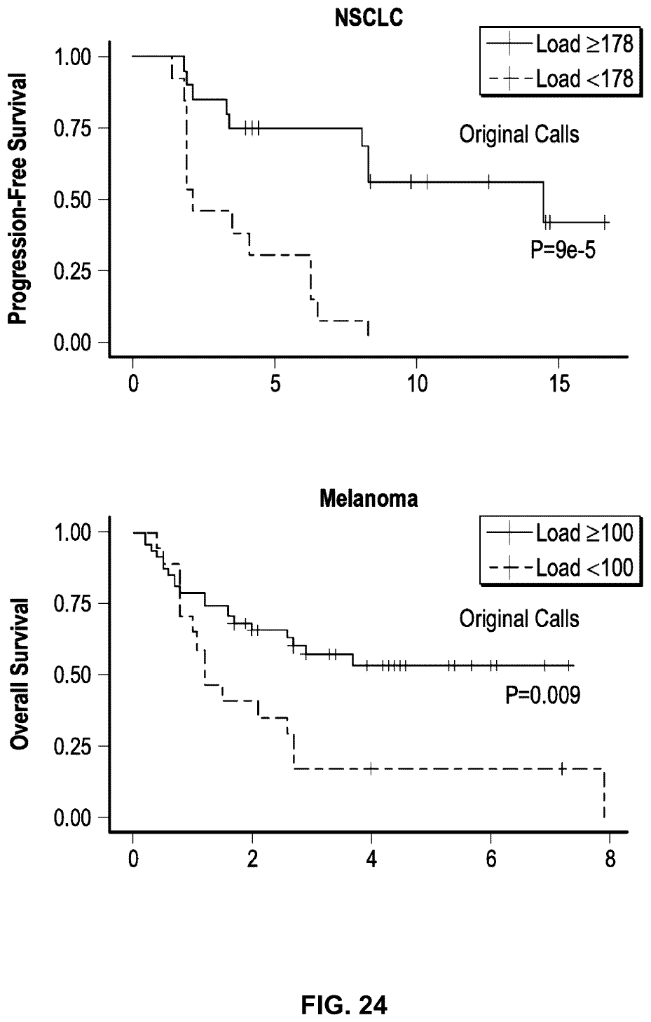

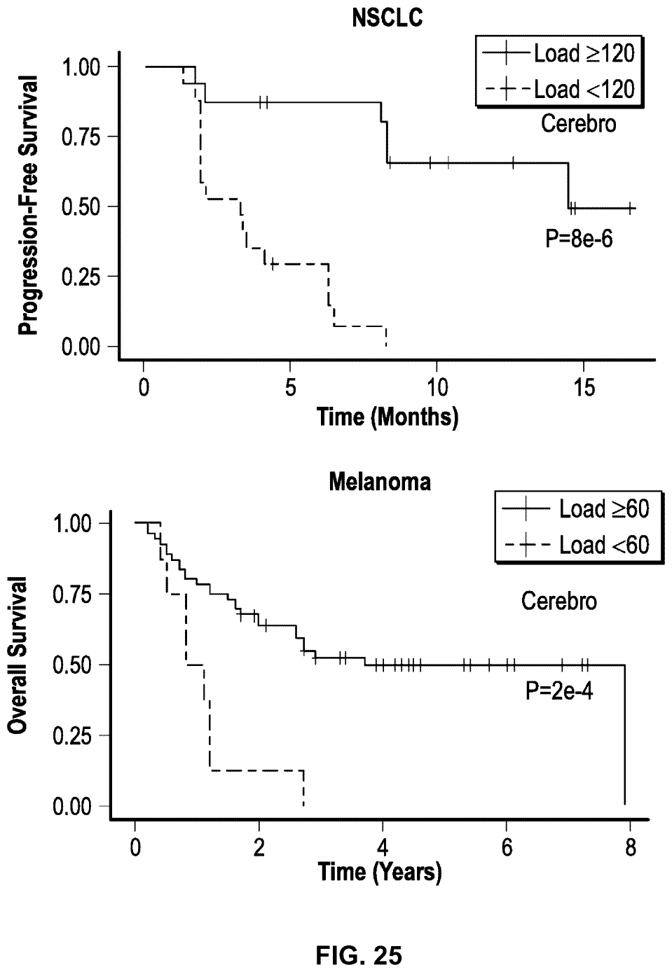

FIGS. 19-22 present an analysis of tumor mutational burden in patients treated with checkpoint blockade. Comparison of Cerebro mutation calls with published calls associated with NSCLC (left panels) or melanoma (right panels). FIG. 22 shows the unique/shared mutation status for all patients. FIG. 23 reports problematic mutations unique to original publications annotated by characteristic issue (Annotation=conflicting consequence; TDP<3=tumor distinct pairs less than 3; TBQ<30-tumor base quality less than 30; Unaligned=no alignment of mutated reads to the mutation position; MAF<5%=mutant allele frequency less than 5%). FIG. 24 is a Kaplan-Meier analysis of progression free survival (left) or overall survival (right) using tumor mutation loads from original publications. FIG. 25 is a Kaplan-Meier analysis of same samples using Cerebro mutational loads. Log-rank P-value shown for each survival plot. Among all mutations in the NSCLC set, 48.2% were concordant between Cerebro and the original report, while 61.9% of mutations in the melanoma cohort were concordant (FIG. 22). An in-depth characterization of mutations that were identified in the original publications but that would be considered false positives using Cerebro and found that the large majority of such calls could be attributed to systematic issues such as limited observations of the mutation in distinct read pairs, poor base quality at the mutation position, and inaccurate alignment (FIG. 23) was performed.

Given the association of mutation load with clinical outcome in patients treated with immune checkpoint blockade, Cerebro was used to determine whether the analyses could be used to improve the classification of patients into mutator groups with different clinical outcomes. Cerebro analyses revealed that the average SBS mutational burdens for NSCLC and melanoma groups were 187 and 501, respectively, both substantially different than the original publications. Previously melanoma analyses were performed using SomaticSniper and led to a lower number of detected mutations, consistent with the lower sensitivity observed with this method (FIG. 14 and FIG. 16). Utilizing the individual mutational loads from the analyses, an improved prediction compared to previous mutation calls for progression free survival (P=8e−6 vs P=9e−5; NSCLC) and overall survival (P=2e−4 vs P=0.009; melanoma) (FIG. 24 and FIG. 25) for these patients was observed. Overall, these analyses suggest that improved mutation determination may have even greater impact on clinical outcome to immune checkpoint blockade than previously anticipated.

### Evaluation of Somatic Mutations in Clinical NGS Analyses

To evaluate the effect of somatic mutation detection methods on NGS clinical cancer sequencing tests, replicate analyses of formalin fixed paraffin embedded (FFPE) or frozen tumor samples from 22 lung cancer patients using three separate approaches for mutational profiling: PGDx CancerSELECT 125 which utilized the Cerebro method, as well as the Thermo Fisher Oncomine Comprehensive Assay and the Illumina TruSeq Amplicon-Cancer Panel which use other mutation calling methods was performed. For each sample, adjacent interspersed FFPE sections were evaluated at two CLIA-certified laboratories using these approaches. Samples from three patients could not be analyzed using the TruSeq Amplicon-Cancer Panel due to insufficient DNA and were excluded from comparative analyses. Putative somatic mutations for the remaining 19 patients were used for concordance evaluation and were limited to genomic regions comprising 16.5 kbp common to all three approaches. Mutations were considered true positives if they were detected in two or more of the three assays, or if identified in only one of the assays and independently confirmed using ddPCR.

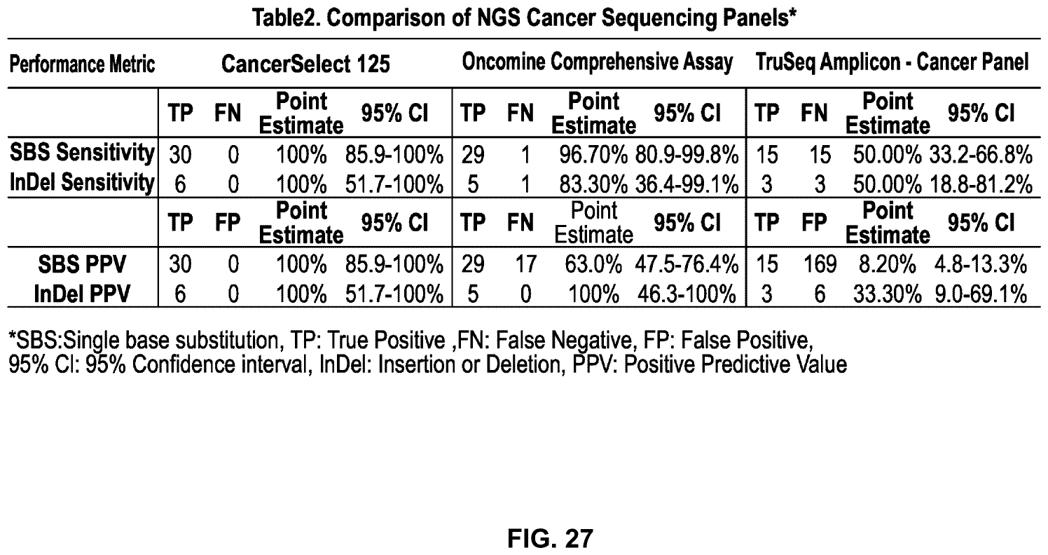

These analyses resulted in a set of true somatic mutations consisting of 30 single base substitutions (SBSs) and six insertions and deletions (indels) in commonly analyzed regions among the 19 patients (Table 2 in FIG. 27). The CancerSELECT 125 panel using Cerebro achieved 100% sensitivity for both SBSs and indels. The Oncomine assay resulted in a sensitivity of 97% for SBSs and 83% for indels. The TruSeq panel was unable to detect 15 SBSs and three indels resulting in a lower sensitivity of 50% for both types of alterations (Table 2 in FIG. 27). CancerSELECT 125, Oncomine and TruSeq approaches resulted in 0, 17, and 175 false positives, respectively. Seventeen false positive SBSs reported by the Oncomine or TruSeq panels were single nucleotide polymorphisms (SNPs) that could be identified in databases of known germline variants and had been removed by the Cerebro approach. However, the large number of false positive results identified by the TruSeq panel suggests additional underlying technical causes, including PCR artifacts and sequencing error. The high number of false positive SBSs from the TruSeq panel dramatically reduced its positive predictive value to 8% (Table 2 in FIG. 27). The CancerSELECT 125 mutation detection approach outperformed Oncomine by achieving higher sensitivity for both SBSs and indels and higher PPV for SBSs, while the TruSeq approach had poor overall performance, including limited sensitivity for both types of alterations and a high false positive rate. Due to the high number of TruSeq non-germline false positives, a detailed evaluation of these candidate mutations in the NGS data obtained using the CancerSELECT 125 platform was performed, but no evidence of these mutations predicted by the TruSeq approach was found. Additionally, comparison of CancerSELECT 125 NGS data using the two mutation callers suitable for tumor-only analyses demonstrated superior sensitivity and positive predictive values for Cerebro. Overall, these analyses reveal the importance of high accuracy somatic mutation detection for identification of bona fide alterations in clinical NGS assays.

### B. Discussion

These studies describe the development of a machine learning approach for optimizing somatic mutation detection in human cancer. These analyses demonstrate that high accuracy mutation detection can improve identification of bona fide alterations to determine total mutational burden for the prediction of outcomes to immunotherapy, as well as to detect of alterations in potentially actionable driver genes. These data highlight the challenges of detecting somatic sequence alterations in human cancer, and provide a broadly applicable means for detecting such changes that is more accurate than existing approaches.

The assessment of mutation calling approaches revealed that existing methods for somatic mutation detection may be significantly influenced by factors that can lead to excessive false positive and negative changes. The machine learning approach described here identified key features in NGS sequence data to minimize false positive calls and to improve sensitivity for bona fide alterations. Coding regions within exome or targeted analyses with >150× coverage were the primary focus. Although Cerebro could be used for improved analyses in these settings similar to other methods, the amount of whole genome sequencing needed to overcome issues of normal tissue contamination and subclonal alterations have resulted in whole exome or targeted analyses remaining one approach for cancer sequence analyses, particularly in the clinical setting.

Given the fundamental importance of somatic genomic alterations in human cancer, the improvements developed here are likely to have significant implications for research and clinical analyses. The re-analysis of TCGA and exome data from patients treated with cancer immunotherapy have identified that a substantial fraction of existing mutation calls are likely to be false positive changes associated with low quality evidence, and that many true alterations may have been missed in current databases. It is estimated that 16% of alterations are likely inaccurate in current TCGA mutation databases and an additional 10% of true alterations may have been missed in these data sets. If these ratios are accurate across TCGA (with −2 million somatic mutations across 10,000 exomes), then the overall number of false positive and negative changes in TCGA is likely to be >500,000. Such discrepancies are likely to be important across a variety of additional efforts, as much of TCGA has been incorporated into mission critical databases such as COSMIC, gnomAD/ExAC, Genomic Data Commons and the International Cancer Genome Consortium.

These analyses are likely to have significant implications for therapies utilizing genomic information, including targeted therapies and immune therapy approaches targeting mutation associated neo-antigens. Improved discrimination of bona fide alterations will facilitate development of mutation-load based predictive biomarkers for immune checkpoint blockade in as well as for understanding changes in cancer genomes during immune therapy. Development of mutation-specific vaccines and immune cell-based therapies will require high confidence identification of alterations that may be unique to individual patients.

These studies have implications beyond analyses of tumor tissues, including for example in deep sequencing of cell-free DNA (cfDNA) to identify somatic mutations in the circulation. Current approaches for cfDNA analyses utilize sequence data with over-30,000× coverage to identify alterations with concentrations as low as 0.05%. Highly accurate analyses of such sequences will provide robust differentiation of true positive and true negative data, especially in cases without prior knowledge of sequence alterations from tumor tissue.

These findings highlight the impact of several underappreciated dimensions of genomic data and support the notion that clinical NGS tests will require rigorous standardization and critical evaluation from initial sample preparation through digital interpretation. If validated effectively, these approaches have the ability to expand options for the treatment and management of patients with cancer.

### C. Materials and Methods

Processing of Exome Data with Cerebro

Reads from material sequenced were adapter masked and demultiplexed using bcl2fastq. All read data were aligned with BWA-MEM and Bowtie2 to a hg19 reference assembly of the human genome with unplaced and unlocalized contigs and haplotype chromosomes removed. Then, Cerebro identified candidate somatic mutations by examining alignments in the tumor and matched normal samples. Alignment data were filtered to remove non-primary alignment records, reads mapped into improper pairs, and reads with >6 edits. Individual bases were excluded from mutant coverage calculation if their Phred base quality was <30 in tumor samples and <20 in normal samples. Only candidate somatic variants found in both pairs of alignments (BWA-MEM and Bowtie2) were scored using our confidence scoring model. Candidate variants with somatic confidence scores <0.75, <3 distinct mutant fragments in the tumor, <10% MAF in the tumor, or <10 distinct coverage in the normal sample were removed. For the analysis of cancer immunotherapy response-associated, mutations between 5-10% MAF were included to compensate for the low tumor purity that appeared to be present in some samples.

For mutations found in at least 50 samples according to the COSMIC v72 database (“hotspots”), relaxed cutoffs were applied. For such hotspot mutations, if their Phred base quality was <20, the bases were excluded in the tumor sample. Also, candidate hotspot mutations were only removed if they had somatic confidence scores <0.25, <2 distinct mutant fragments in the tumor, or <5% MAF in the tumor. Because sequence data obtained from TCGA were often less than 100 bp in length, which was found to reduce Bowtie2 alignment sensitivity for long indels, a set of relaxed cutoffs for hotspots that were indels >8 bp in length were created. These relaxed indel hotspot filtering criteria focused only on the BWA-MEM alignments, and removed mutations with <5 distinct fragments in the tumor, a left-tailed FET p-value >0.01, <5% MAF in the tumor, or any mutant fragments in the normal sample.

Variants were further filtered for coding consequence using VEP and CCDS/Refseq removing intragenic and synonymous mutations. Finally, variants that were listed as Common in dbSNP version 138 were removed.

Processing of Exome Data with External Variant Callers

Read data were aligned with BWA-MEM to a hg19 reference assembly of the human genome with unplaced and unlocalized contigs and haplotype chromosomes removed. The Picard MarkDuplicatesWithMateCigar program was used on the resulting BAM files to find optical and PCR duplicates. Each external variant caller was run with default parameters. In the case of Strelka, the reported “tier 2” set of variants were used. Similarly to the processing with Cerebro, for all variant callers, variants with MAF <10%, as well as intragenic and synonymous mutations, and variants listed as Common in dbSNP138 were removed. Variants failing a caller's default set of filters were also removed. For VarDict, variants that hit either of two filters suggested by one of VarDict's authors were removed.

### Confidence Scoring Model and Training

A normal cell line (CRL-2339, ATCC) derived from peripheral blood that had undergone sample preparation and exome capture was sequenced twice. One of these sequencing runs was designated the “training tumor” and had novel variants spiked into it using BAMSurgeon. Novel coding variants were randomly generated across the exome, at MAFs ranging from 1.5625% to 100%, and were a mixture of substitutions, insertions, and deletions. The range of MAFs used for training was intended to begin well below the expected limit of detection (5% or 10% MAF) to ensure that calls near the limit would be accurate. Novel indels ranged from 1 to 18 bp in length. Additional novel indels were spiked in by locating polynucleotide tracts within the exome and inserting 1 or 2 repeat unit contractions or extensions of the tracts. After spiking the training tumor with BAMSurgeon, the read data from the training tumor were realigned with both BWA-MEM and Bowtie2, and then all candidate somatic variants supported by at least one tumor read were reported using Cerebro. Candidate somatic variants found in both pairs of alignments formed the training set for the scoring model.

For each candidate somatic variant, Cerebro reports several alignment and sequence quality statistics. Each of these are calculated for the two sets of alignments and are concatenated together to form a feature vector for a candidate somatic variant. The training set for the scoring model consists of the feature vectors for each candidate variant, and a labelling indicating whether or not the variant was spiked into the training tumor (i.e., if the candidate is a somatic variant or not). The scoring model itself is an extremely randomized trees model with 1000 decision trees, implemented using the scikit-learn library (see Pedregosa et al., Scikit-learn: Machine learning in Python. Journal of Machine Learning Research 12, 2825-2830 (2011), incorporated by reference), with the reported confidence score being the percentage of the model's trees that would classify the variant as somatic.

### Validation of Somatic Variants

Five matched tumor/normal breast cancer cell line pairs in which several hundred somatic variants had previously been identified and validated through Sanger sequencing analyses were evaluated. To add to this set, exome sequencing of the cell lines using the Illumina HiSeq 2500 was performed. Then the Illumina data was analyzed using 3 variant calling programs (VarDict, MuTect 1, and the Cerebro pipeline). Somatic variants called by all three programs were considered to be validated. Somatic variants called by 1 or 2 of those programs with a reported MAF of at least 20% (by at least one program) were visually inspected for alignment artifacts. Those variants passing visual inspection and having at least 10 reads covering the locus in the normal sample were then tested using digital droplet PCR, and variants validated by ddPCR formed the remainder of the validated variant set.

### Evaluation of Variant Caller Accuracy

For simulated datasets, true positives (TP) were those spiked-in somatic variants found by a program, false positives (FP) were variants called by a program that were not spiked-in, and false negatives (FN) were spiked-in variants not called by a program. For these simulated datasets, sensitivity is defined as TP/(TP+FN), positive predictive value (PPV) as TP/(TP+FP), and false positive rate is the number of false positives reported per megabase of the exome (51.5 Mbp). For the cell line datasets, a validated variant set as described above was created, and selected those variants called by at least one caller as having a MAF of at least 20%; these selected variants created the validated comparison set. When evaluating the variant callers on cell line data: TP were comparison variants found by a program; FP were variants not in the comparison set that were called by a program with a MAF of at least 20%; and FN were comparison variants not found by a program. Because variants reported to have a MAF of at least 20% were validated, the PPV for the cell line data is defined as X/(X+FP), where X is the number of TP variants that a program claimed had a MAF of at least 20%. This approach compensates for the variation in reported allele frequency between variant calling programs by restricting PPV calculation to only those variants that a program reported as being over the validation MAF threshold of 20%.

### Simulation Experiments

Three simulation experiments were performed, designed to evaluate the accuracy of the various variant calling programs. In each experiment, a set of simulated tumor/normal pairs created by sequencing 6 exome-captured normal cell lines twice to create 6 sample pairs was used; one of the samples in each pair was designated the “tumor”, and both samples were aligned to the human genome using Bowtie2. Depending on the experiment, a set of artificial coding somatic variants were inserted into the tumors using BAMSurgeon. After BAMSurgeon was run, the read sequence data was extracted from the modified BAM file, and the resultant FASTQ data were aligned again using the methods described in the “Processing of exome data with Cerebro” and “Processing of exome data with external variant callers” sections above.

The first simulation experiment was designed to simulate low purity tumors, with 120 single base substitutions and 12 indels inserted into each tumor at MAFs ranging from 10-25%. In this final simulation, several accuracy metrics, including sensitivity, PPV, FPR, and F-score were evaluated. The second experiment was solely a test of specificity, where no somatic variants were inserted into the simulated tumor; this examination of somatic variant calling specificity with technical replicates is similar to that discussed by Saunders et al. in their presentation of Strelka (see Saunders et al., Strelka: accurate somatic small-variant calling from sequenced tumor-normal sample pairs. Bioinformatics 28, 1811-1817 (2012), incorporated by reference).

In the final simulation experiment, in which the focus was on sensitivity, 7,000 coding variants were inserted, consisting of 1,000 single base substitutions and 6,000 indels (1,000 each of 1, 2, & 3 bp insertions, and 1, 2, & 3 bp deletions). The second experiment's variants were inserted at MAFs ranging from 10-100%.

ddPCR Methods

Droplet digital PCR forward and reverse primers as well as wild type and mutant probes were created using the Bio-Rad ddPCR Custom Design Portal. Genomic DNA corresponding to 10,000 genome equivalents was added to 10 μL 2× ddPCR Supermix (Bio-Rad), 1 μL 20× target primers and probe (FAM), 1 μL 20× reference primers and probe (HEX), and brought to a 22 μL volume with nuclease free water to create a reaction mix. A DG8 cartridge in a DG8 cartridge holder (Bio-Rad) was loaded with 20 μL of reaction mix and 70 μL Droplet Generation Oil (Bio-Rad). The cartridge was placed in a QX200 Droplet Generator to generate approximately 20,000 nanoliter sized droplets. Droplets were loaded into a twin-tec 96 well, semi skirted plate (Eppendorf) and sealed with a foil heat seal using a PX1 PCR Plate Sealer (Bio-Rad).

Subsequent PCR cycling was performed on a C1000 Touch Thermal Cycler (Bio-Rad) with the following conditions: 95° C. for 10 minutes, followed by 40 cycles of 94° C. for 30 seconds and 55° C. for 1 minute, and ending with 98° C. for 10 minutes. The plate was then loaded on a QX200 Droplet Reader (Bio-Rad) and PCR positive and negative droplets were quantified. Raw droplet data was analyzed with QuantaSoft software (Bio-Rad). Thresholds were manually assigned using 2D amplitude clustering plots and the crosshair tool for each tumor and normal pair. Tumor samples were run in duplicate, and the average mutant allele fraction was taken. For the comparative evaluation of clinical targeted sequencing panels, there were some modifications to the ddPCR protocol. 5,500 genome equivalents were used, and 1 μL of a 20× mixture of target primers and probes and reference primers and probes (FAM and HEX respectively) were used (Bio-Rad). As there were no matched normal samples, wild type and mutant oligomers were designed as controls for each target investigated (Operon Biotechnologies).

### WES Data Extraction and Annotation

The results shown here are in part based upon data generated by the TCGA Research Network as outlined in the TCGA publications guidelines. TCGA WES datasets (bam alignment files) represented untreated primary tumors and paired normal tissue samples obtained from the Cancer Genomics Hub. WES-derived somatic mutation calls were obtained from the MC3 Project TCGA somatic mutation calls (v0.2.8) were also obtained from the Synapse repository.

Variant supporting coverage and total coverage were extracted and manually reviewed for consistency. To normalize across cancer types, mutations with fewer than three variant-associated reads or less than 10% mutant allele frequency were filtered prior to downstream comparative analysis. For mutations found in at least 50 samples according to the COSMIC v72 database (“hotspots”), a 5% minimum mutant allele frequency was allowed. Additional somatic mutation call sets generated by MuTect2 were downloaded from the Genomic Data Commons and also Broad GDAC Firehose using the firehose get download client prioritizing the BIFIREHOSE Oncotated Calls somatic mutation call sets compiled from various TCGA Genome Sequencing Centers' bioinformatics pipelines. For comparisons of Cerebro to other call sets, included mutations were required to fall within a common ROI set. The source of the primary mutation calling tools used for each cancer type may be found in the corresponding TCGA marker publications. Concordance analysis of somatic mutations from 66 oncogenes and tumor suppressor genes included manual review to determine shared status of mutations within the same or adjacent codons. Whole-exome melanoma or NSCLC immunotherapy datasets are available via NCBI dbGaP (accessions phs000980 and phs001041).

### Clinical Study Design and Analysis

Twenty-two total samples, seventeen formalin fixed paraffin embedded (FFPE) and five frozen tumor tissue specimens obtained from lung cancer patients were procured from ILSBio/Bioreclamation and analyzed for the presence of sequence mutations using three targeted cancer gene panels from independent vendors. All samples were obtained under Institutional Review Board-approved protocols with informed consent for research use at participating institutions. One set of patient samples was processed and analyzed for sequence mutations by Personal Genome Diagnostics (Baltimore, MD) using the CancerSELECT 125 panel. In brief, samples were reviewed by a pathologist to determine the percent tumor purity followed by macro-dissection and DNA extraction. DNA was fragmented and used for CancerSELECT 125 library preparation and capture. Libraries were sequenced using HiSeq2500 instruments (Illumina). Sequencing output was analyzed and mutations identified using Cerebro. An identical set of FFPE and frozen tumor tissue specimens were sent to MolecularMD (Portland, OR) along with a hematoxylin and eosin stained image for processing and next-generation sequencing analysis using two cancer specific panels supplied by two independent vendors: Oncomine Comprehensive Assay (ThermoFisher) and TruSeq Amplicon-Cancer Panel (Illumina). To limit the effects of tumor heterogeneity on analysis, slides were distributed in non-sequential order for testing. For orthogonal analysis, comparisons were limited to regions of interest (ROI) that were included in all three panels. Samples that failed quality check in one or more panels were excluded. A sequence mutation was considered a True Positive (TP) if there was positivity in at least two panels and a False Positive (FP) if only detected in one panel. Sequence mutations were considered True Negatives (TN) if there was negativity in at least two panels. A position with no mutation detected was considered a False Negative (FN) in a panel if that position was concordantly positive in the other two panels. Genomic positions that were masked based on known single nucleotide polymorphisms (SNPs) in the CancerSELECT 125 panel were considered FP in Oncomine and TruSeq analyses. As there were >150 FPs detected with the TruSeq panel, discordant resolution was limited to FPs or FNs obtained by CancerSELECT 125 and Oncomine (not considered SNPs) and were resolved using ddPCR.

### Statistical Methods

The Mann-Whitney U test was employed to compare quantitative measures (e.g., nonsynonymous mutational load) between groups of interest. Comparisons of relative frequencies utilized Fisher's exact test. The log-rank test was used to evaluate significant differences between Kaplan-Meier curves for overall survival or progression free survival in the Melanoma (see Snyder et al., Genetic basis for clinical response to CTLA-4 blockade in melanoma, N Engl J Med 371, 2189-2199 (2014), incorporated by reference) or NSCLC (see Rizvi et al., Cancer immunology: Mutational landscape determines sensitivity to PD-1 blockade in non-small cell lung cancer, Science 348, 124-128 (2015), incorporated by reference) immunotherapy datasets, respectively. Confidence intervals (95% CIs) for proportions in clinical NGS comparisons were calculated using the method described by Wilson and Newcombe with no continuity correction (see Newcombe, Two-sided confidence intervals for the single proportion: comparison of seven methods, Stat Med 17, 857-872 (1998) and Wilson, calculating a confidence interval of a proportion, J Am Stat Assoc 22, 209-212 (1927), both incorporated by reference).

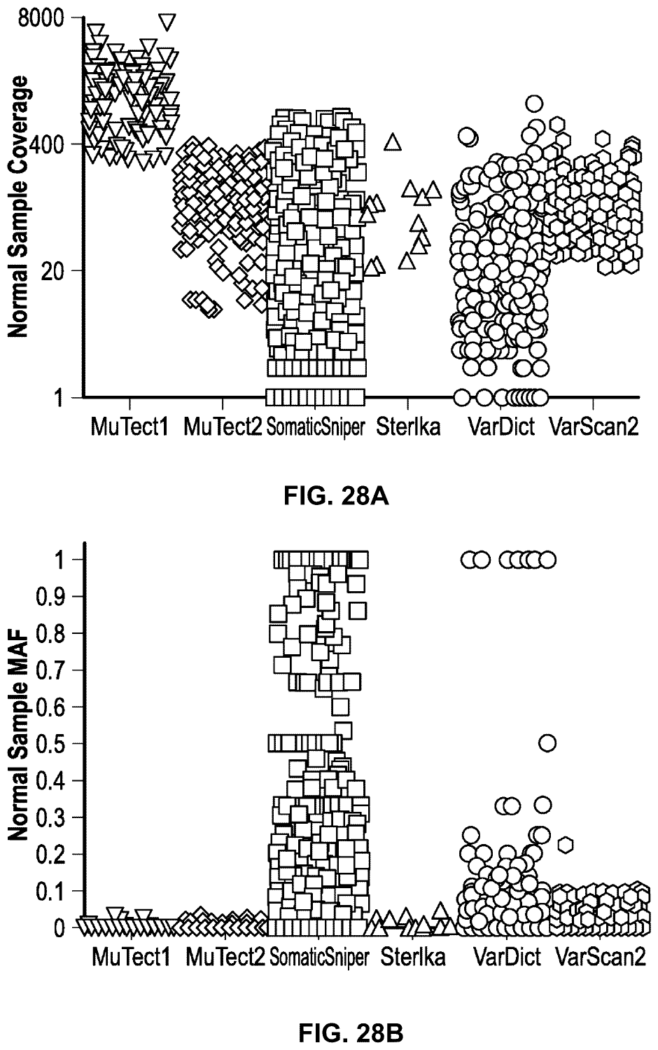

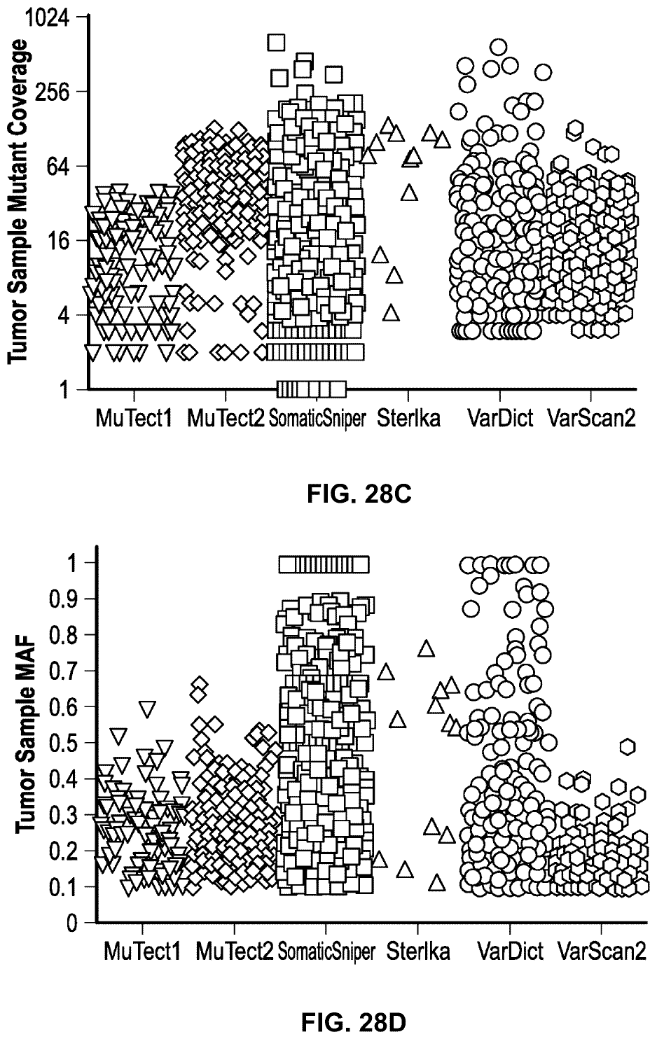

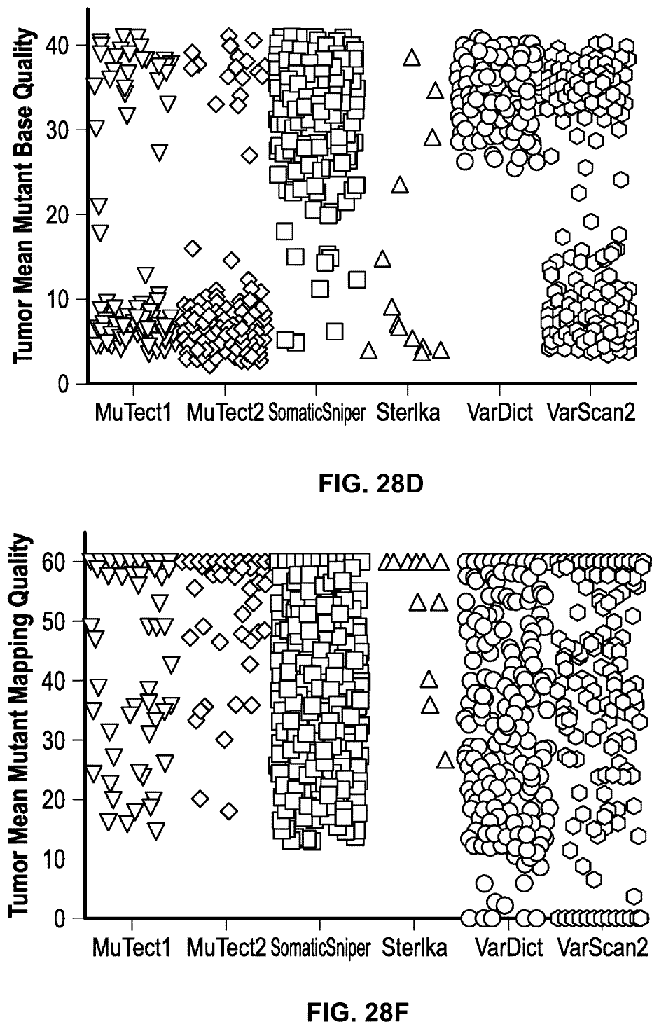

FIGS. 28A-F show false position evaluation for somatic mutation callers. In particular, using results from simulated mutations in exomes, we display factors frequently associated with false positive mutation calls including tumor/normal coverage, tumor/normal mutant allele frequency (MAF), mean mutant base quality and mean mutant mapping quality. Each point shown in the FIGS. represents a false positive mutation called by a specific program.

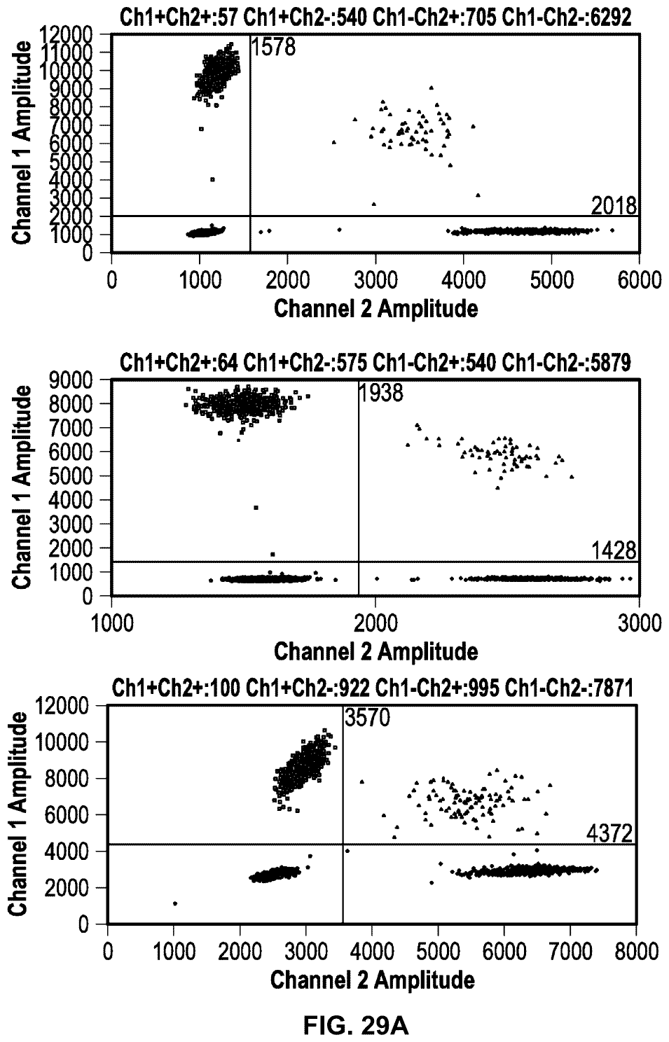

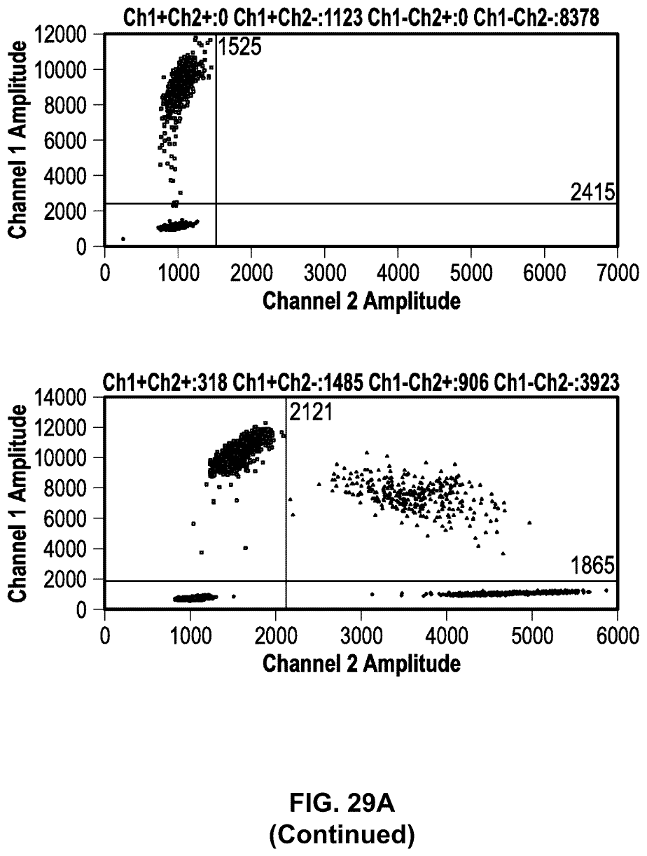

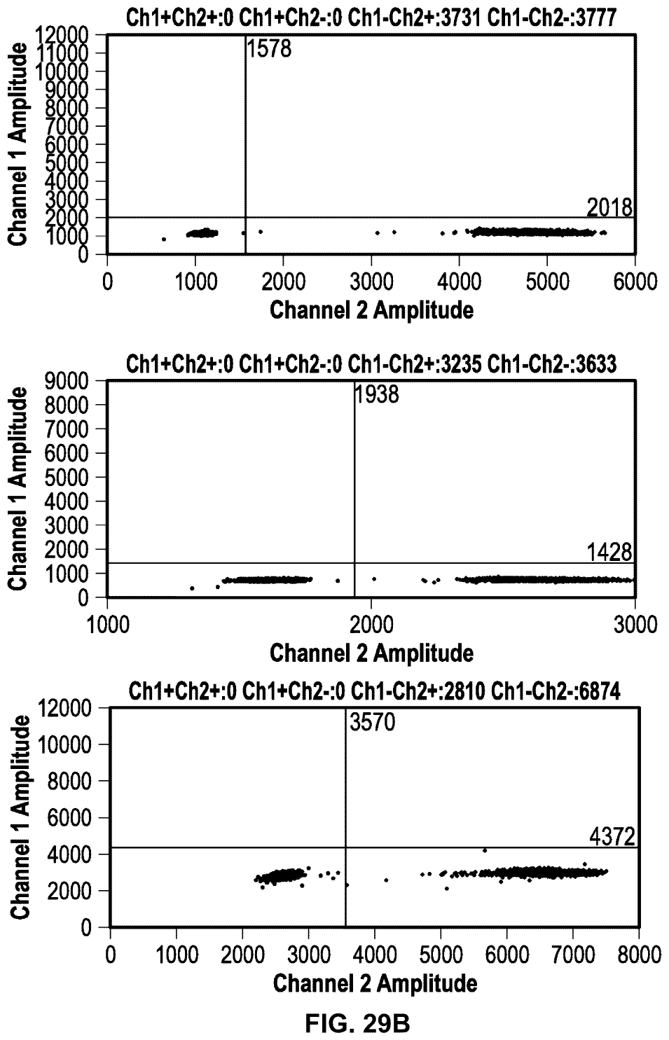

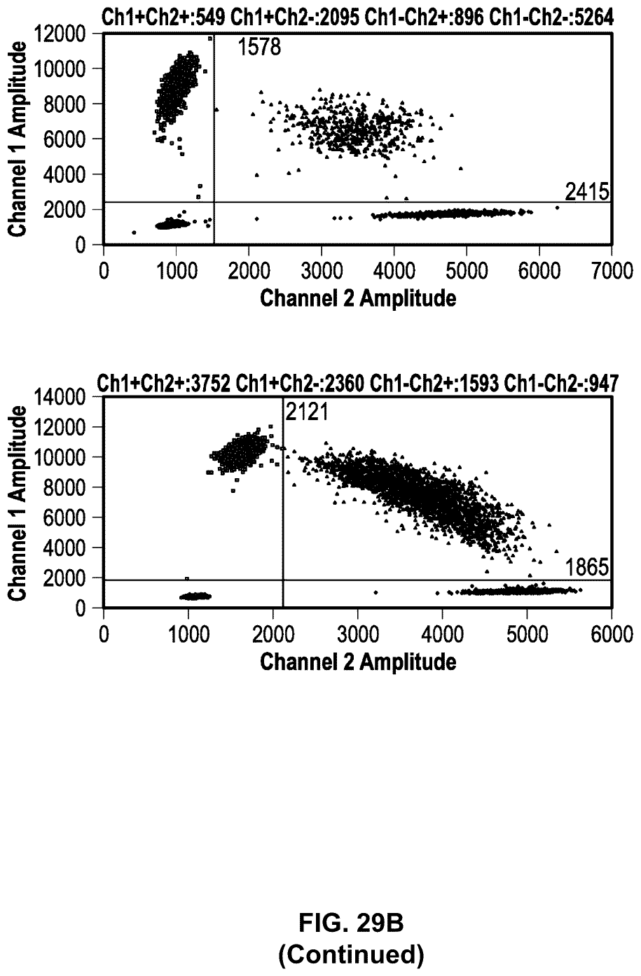

FIG. 29 illustrates the results of droplet digital PCR mutation validity analyses. Each panel in FIG. 29 is a unique sample and each point on the graphs represents the fluorescent signal from PCR amplified fragments in a droplet. Green droplets have been fluorescently tagged with reference a reference probe (HEX), blue droplets have been tagged with a mutant probe (FAM), and orange droplets have been tagged with both mutant and reference probes. Rows correspond to a variant where both the tumor and matched normal sample were tested. (A) Somatic mutations validated by ddPCR. In each case, the tumor shows amplified mutant copies (blue) while the normal only shows amplified reference copies (green). (B) Germline and false positive variants. For the top three rows, mutant copies (blue) amplified in both the tumor and matched normal sample, meaning these variants are germline. The bottom two variants have only reference amplification (green) in both the tumor and normal, meaning these variants were false positive calls by NGS.

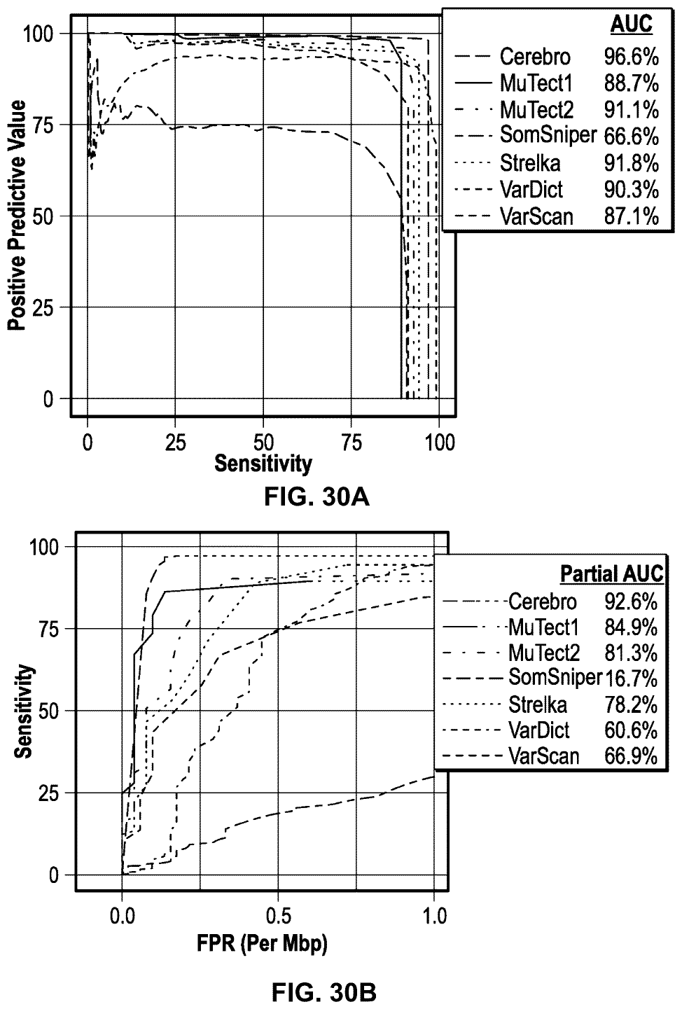

FIGS. 30A and 30B illustrate curve analyses for Cerebro and other mutation callers using experimentally validated alterations. In particular, precision-recall and ROC curve analyses of Cerebro and other mutation callers using experimentally validated alterations are shown. Cerebro outperforms other methods using both precision-recall (A, sensitivity vs. positive predictive value) and ROC analyses (B, false positive rate (per Mbp) vs. sensitivity).

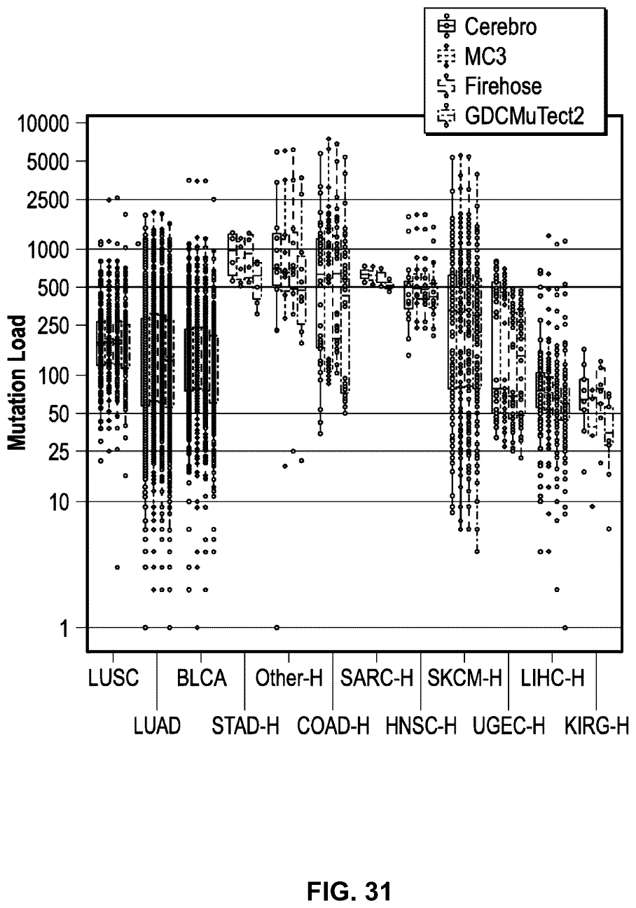

FIG. 31 illustrates mutation loads for TCGA exomes using different mutation calling methods. The call sets include Cerebro, MC3 (PanCanAtlas), Broad Firehose, and MuTect2 (Genomic Data Commons). The acronyms along the bottom of the chart are LUSC=lung squamous cell carcinoma; LUAD=lung adenocarcinoma; BLCA=bladder; STAD=stomach; COAD-colorectal; SARC=sarcoma; HNSC-head and neck squamous cell; SKCM=melanoma; UCEC-uterine; LIHC-liver; KIRC=kidney; *-H=set enriched for high mutation load samples.

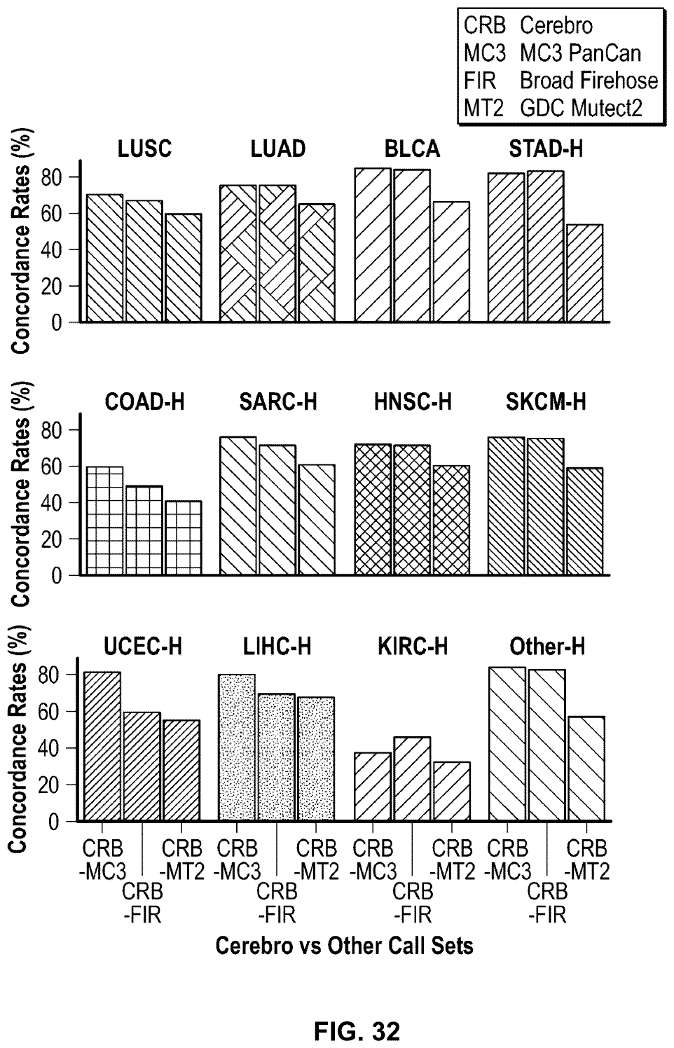

FIG. 32 illustrates concordance rates (percentage of total mutations) of Cerebro compared to other mutation call sets for TCGA exomes. The acronyms along the bottom of the chart are LUSC=lung squamous cell carcinoma; LUAD=lung adenocarcinoma; BLCA=bladder; STAD=stomach; COAD=colorectal; SARC=sarcoma; HNSC=head and neck squamous cell; SKCM=melanoma; UCEC-uterine; LIHC=liver; KIRC=kidney; *-H=set enriched for high mutation load samples.

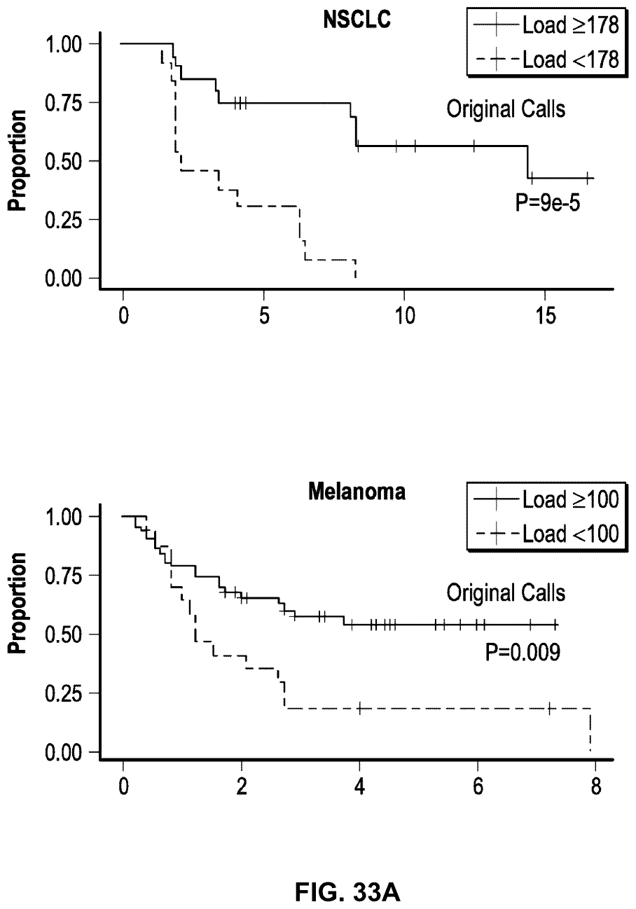

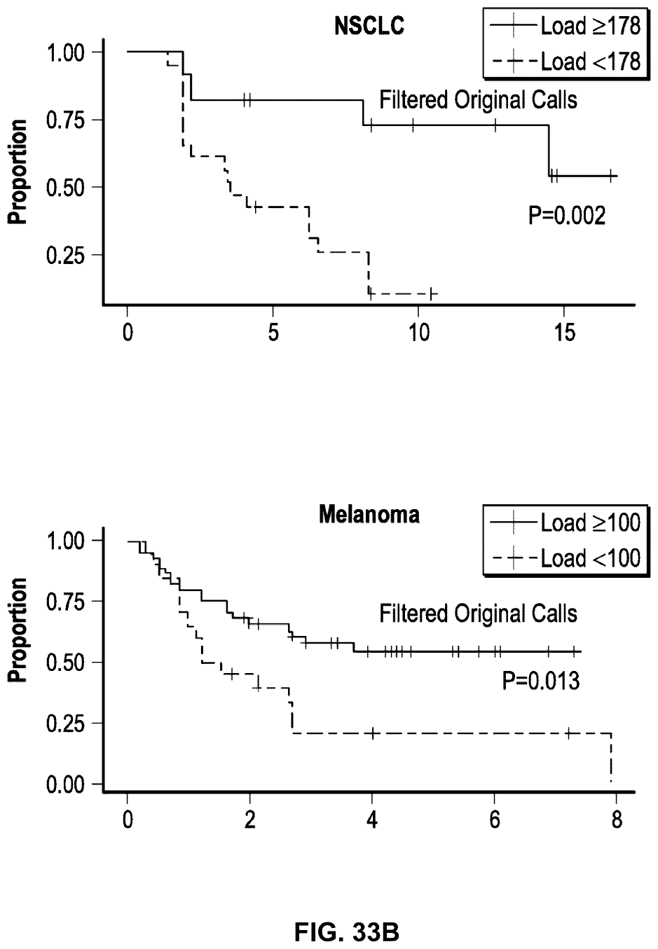

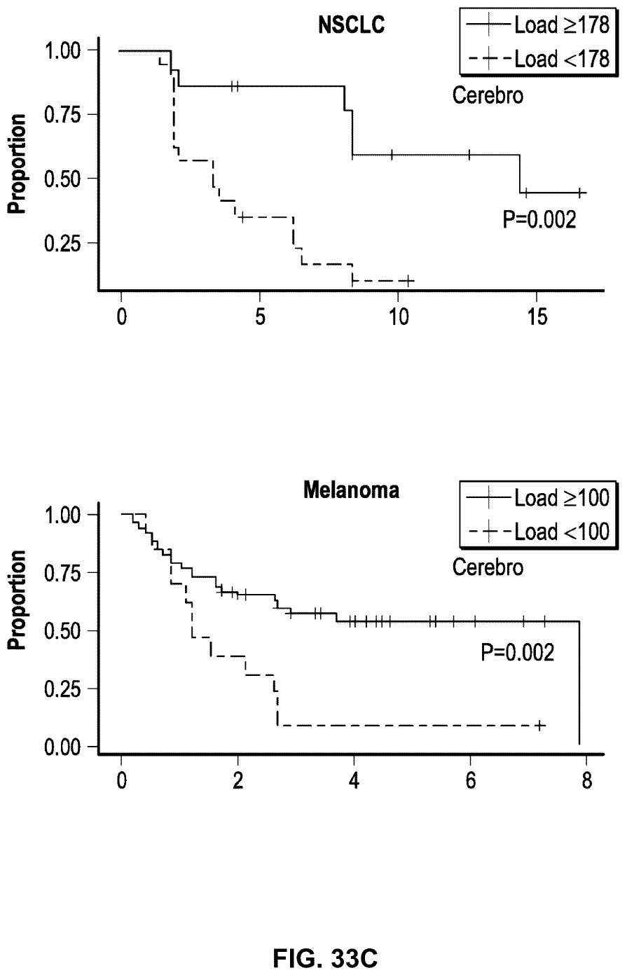

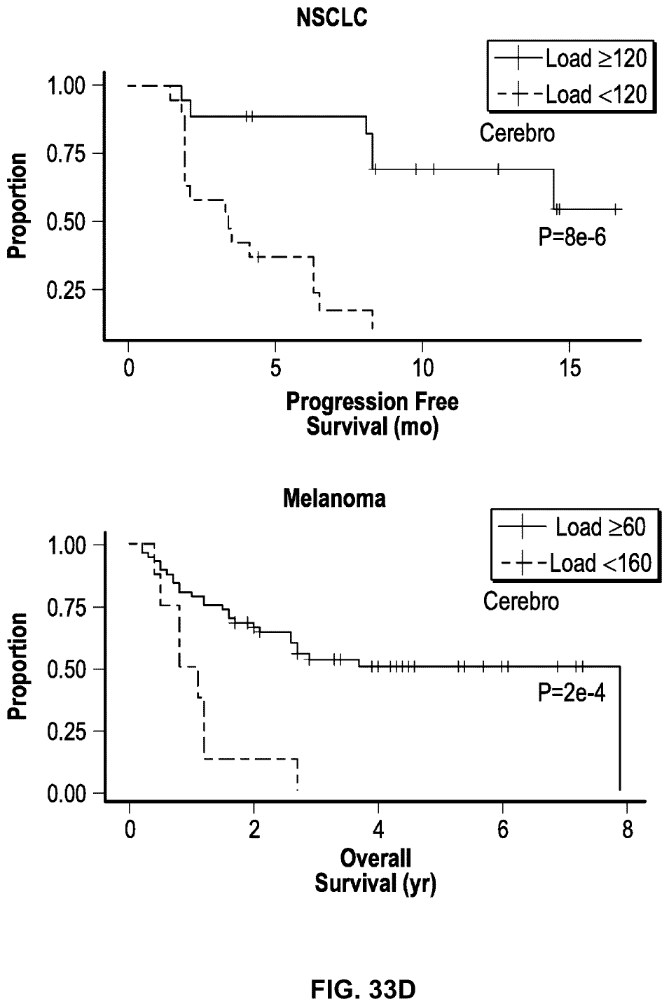

FIGS. 33A-33D illustrate comparison of Cerebro mutation calls with published calls and a response to checkpoint inhibitors associated with mutational load. The figures show a comparison of Cerebro mutation calls with published calls associated with NSCLC (left panels) or melanoma (right panels). (A) Kaplan-Meier analysis of progression free survival (left) or overall survival (right) using tumor mutation loads from original publications; (B) original publication results filtered for problematic mutations; (C) Cerebro calls using same threshold as original publications; (D) Cerebro calls using optimal thresholds for survival prediction. Log-rank P-value shown for each survival plot.

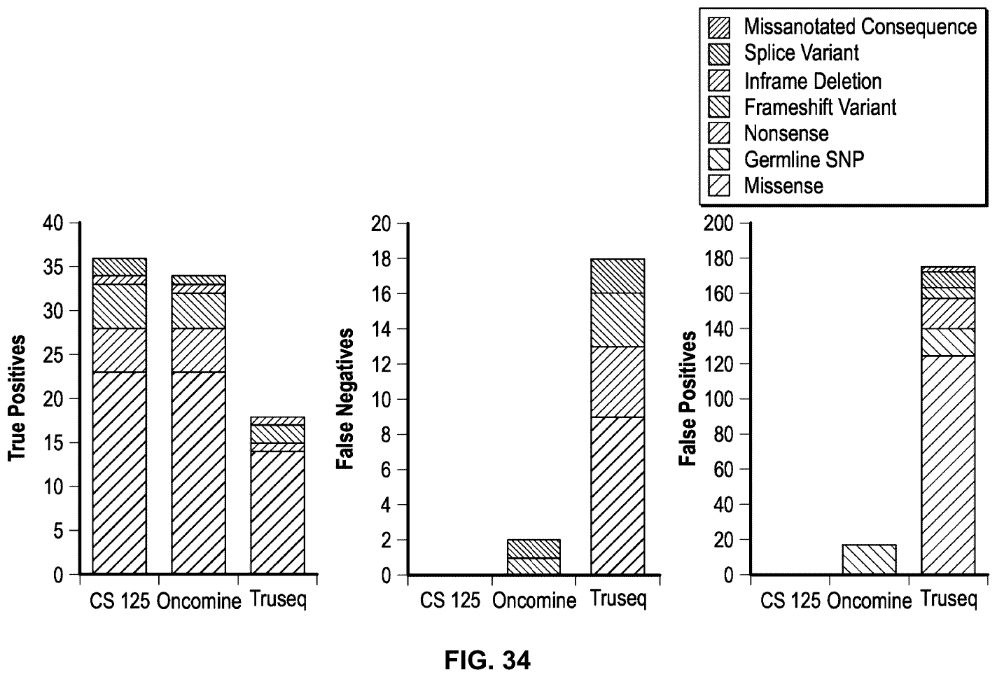

FIG. 34 illustrates comparative results of three clinical sequencing panels. The rreported results include true positives, false negatives and false positives.

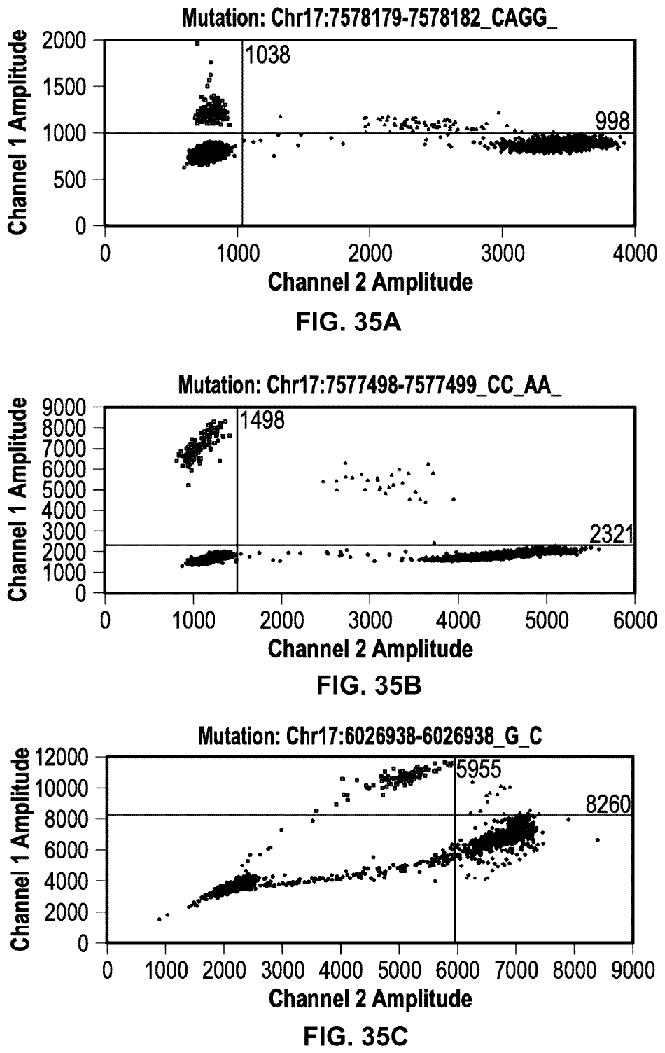

FIGS. 35A-35C illustrate alterations confirmed by ddPCR in a clinical panel comparison. Each panel is a unique sample and each point on the graphs represents the fluorescent signal from PCR amplified fragments in a droplet. Green droplets have been fluorescently tagged with reference a reference probe (HEX), blue droplets have been tagged with a mutant probe (FAM), and orange droplets have been tagged with both mutant and reference probes.

NGS Analysis of Plasma Derived cfDNA and Contrived DNA

cfDNA obtained from plasma samples were quantified using the Qubit dsDNA High-Sensitivity Assay (Thermo Fisher, USA). Whole genome next generation sequencing libraries were prepared from cell-free DNA using a target of 10 ng of DNA through end-repair, A-tailing, and adapter ligation with custom molecular barcoded adapters. Subsequently, these libraries were amplified through 5 cycles of PCR, pooled, and sequenced with 150 bp paired end reads using the Illumina NovaSeq6000 platform (Illumina, USA) to a target depth of 30λ. After demultiplexing was performed, FASTQ files were quality trimmed using Trimmomatic and aligned to the hg19 human reference genome using BWA-MEM2. Somatic variant identification was performed using VariantDx, which has demonstrated high accuracy for somatic mutation detection and differentiating technical artifacts to enable analyses of SNVs.

### V. Additional Considerations

Although specific examples have been described, various modifications, alterations, alternative constructions, and equivalents are possible. Examples are not restricted to operation within certain specific data processing environments but are free to operate within a plurality of data processing environments. Additionally, although certain examples have been described using a particular series of transactions and steps, it should be apparent to those skilled in the art that this is not intended to be limiting. Although some flowcharts describe operations as a sequential process, many of the operations may be performed in parallel or concurrently. In addition, the order of the operations may be rearranged. A process may have additional steps not included in the figure. Various features and aspects of the above-described examples may be used individually or jointly.

Further, while certain examples have been described using a particular combination of hardware and software, it should be recognized that other combinations of hardware and software are also possible. Certain examples may be implemented only in hardware, or only in software, or using combinations thereof. The various processes described herein may be implemented on the same processor or different processors in any combination.

Where devices, systems, components or modules are described as being configured to perform certain operations or functions, such configuration may be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes may communicate using a variety of techniques including but not limited to conventional techniques for inter-process communications, and different pairs of processes may use different techniques, or the same pair of processes may use different techniques at different times.

Specific details are given in this disclosure to provide a thorough understanding of the examples. However, examples may be practiced without these specific details. For example, well-known circuits, processes, algorithms, structures, and techniques have been shown without unnecessary detail in order to avoid obscuring the examples. This description provides example examples only, and is not intended to limit the scope, applicability, or configuration of other examples. Rather, the preceding description of the examples will provide those skilled in the art with an enabling description for implementing various examples. Various changes may be made in the function and arrangement of elements.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific examples have been described, these are not intended to be limiting. Various modifications and equivalents are within the scope of the following claims.

In the foregoing specification, aspects of the disclosure are described with reference to specific examples thereof, but those skilled in the art will recognize that the disclosure is not limited thereto. Various features and aspects of the above-described disclosure may be used individually or jointly. Further, examples may be utilized in any number of environments and applications beyond those described herein without departing from the broader spirit and scope of the specification. The specification and drawings are, accordingly, to be regarded as illustrative rather than restrictive.

In the foregoing description, for the purposes of illustration, methods were described in a particular order. It should be appreciated that in alternate examples, the methods may be performed in a different order than that described. It should also be appreciated that the methods described above may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, floppy diskettes, ROMs, RAMS, EPROMs, EEPROMs, magnetic or optical cards, flash memory, or other types of machine-readable mediums suitable for storing electronic instructions. Alternatively, the methods may be performed by a combination of hardware and software.

Where components are described as being configured to perform certain operations, such configuration may be accomplished, for example, by designing electronic circuits or other hardware to perform the operation, by programming programmable electronic circuits (e.g., microprocessors, or other suitable electronic circuits) to perform the operation, or any combination thereof.

What is claimed is:

1. A method comprising:
performing whole genome sequencing using a high-throughput sequencing system on nucleic acids from a sample obtained from a subject to generate raw data, wherein the raw data is stored in a specific format in a storage medium associated with the high-throughput sequencing system or in a local storage device communicatively coupled to the high-throughput sequencing system;
transmitting the raw data from the high-throughput sequencing system to a computing system, wherein the computing system comprises at least one processor and a memory, wherein the computing system is either a local server connected to the high-throughput sequencing system or a cloud-based server accessible via a network;
processing the raw data by the computing system to generate candidate variants and a set of feature values for each candidate variant of the candidate variants, wherein the processing comprises:
obtaining sequence reads from the raw data;
filtering the sequence reads based on a predetermined protocol to generate filtered sequence reads;
determining the candidate variants based on the filtered sequence reads; and
determining the set of feature values for each candidate variant of the candidate variants based on the filtered sequence reads and the raw data, wherein the feature values are stored in a specific data structure;
processing the feature values by the computing system using a trained machine learning model to identify somatic mutations from the candidate variants, wherein the processing comprises:
loading the trained machine learning model into the memory of the computing system,
allocating memory and computational resources to execute the trained machine learning model, wherein the memory allocation comprises dynamic memory management for model execution, and wherein the computational resource allocation comprises assigning processing tasks to specialized accelerators of the computing system,
inputting the feature values into the trained machine learning model, and
generating, by the trained machine learning model, output data comprising the somatic mutations and associated metadata;
transmitting results based on the somatic mutations from the computing system to an end device, wherein the end device is communicatively connected to the computing system via the network; and
displaying a report on the end device based on the results.

2. The method of claim 1, wherein the specific format is a .bcl file, a .cbcl file, a FASTQ file, a .fast5 file, a .bax.h5 file, a .subreads.bam file, a FASTA file, a .dat file, a .bam file, or a .pod5 file.

3. The method of claim 1, wherein the high-throughput sequencing system generates the raw data, wherein the raw data is divided into data chunks in real time prior to transmitting to the computing system, and wherein tasks comprising base calling are offloaded to the computing system in parallel as the data chunks are received by the computing system.

4. The method of claim 1, wherein the nucleic acids are cell-free DNA (cfDNA) isolated from a liquid sample of the subject.

5. The method of claim 1, wherein the whole genome sequencing is a low-coverage whole genome sequencing (lcWGS), a medium-coverage whole genome sequencing, or a high-coverage whole genome sequencing.

6. The method of claim 1, wherein the specific data structure is a table, a multi-dimensional array, a linked list, a DataFrame, a JSON, a matrix, or a spreadsheet.

7. The method of claim 6, wherein the table is a relational database table or a hash table.

8. The method of claim 1, wherein the trained machine learning model is a random forest model.

9. The method of claim 8, wherein the random forest model comprises at least 500 decision trees.

10. The method of claim 1, wherein the predetermined protocol comprises filtering out all short tandem repeats (STRs) or STRs that fall outside of exons.

11. The method of claim 1, wherein each feature value corresponds to a predetermined feature, and wherein the set of features values corresponds to at least 20 predetermined features.

12. The method of claim 11, wherein the at least 20 predetermined features comprise (i) a count of unique instances where each nucleotide is observed at a specific position in the candidate variant, (ii) a count of sequence reads at a specific position in the candidate variant that show a mutated allele, (iii) a specific statistic of a quality score at a specific position in the candidate variant, (iv) a count of mutant allele pairs observed in forward strand reads at a specific position in the candidate variant, (v) a count of mutant allele pairs observed in reverse strand reads at a specific position in the candidate variant, (vi) a count of sequence reads at a specific position in the candidate variant that show a mutated allele above a cutoff quality score, and (vii) a mutation type.

13. The method of claim 1, wherein the candidate variants comprise at least a 100,000 variants.

14. The method of claim 1, further comprising:
fragmenting the nucleic acids into DNA fragments, wherein each DNA fragment is about 200-500 base pairs;
ligating paired-end adapters to both ends of each DNA fragments; and
amplifying the ligated DNA fragments to generate a DNA library,
wherein the whole genome sequencing is paired-end sequencing on the DNA library.

15. The method of claim 1, further comprising training a machine learning model to select somatic mutations from candidate variants, wherein the training comprises:
obtaining training data, wherein the training data comprises sequencing data from matched tumor-normal samples, wherein the sequencing data comprises labeled variants and a set of feature values for each labeled variant, wherein each labeled variant is labeled as a somatic mutation or a non-somatic mutation;
generating a training set and a validation set from the training data;
inputting the training set into the machine learning model to train the machine learning model to minimize a misclassification error;
validating the machine learning model using the validation set;
in response to a determination that a predetermination standard is not met, tuning hyperparameters of the machine learning model and iteratively performing the inputting and the validating steps; and
in response to a determination that the predetermination standard is met, storing the trained machine learning model in the local server or the cloud server.

16. The method of claim 1, further comprising implementing a multi-tiered memory caching system in the computing system to optimize retrieving and processing the raw data and feature values.

17. The method of claim 16, wherein the multi-tiered memory caching system comprises a priority queue for candidate variants, and wherein the candidate variants are stored based on a confidence level generated by the trained machine learning model.

18. The method of claim 1, wherein displaying the report further comprises integrating the somatic mutations with external clinical databases and knowledge repositories to provide real-time contextual annotations and a recommendation for a personalized treatment plan.

19. The method of claim 18, wherein the contextual annotations include drug-gene interaction information, clinical trial eligibility, and/or prognostic insights to provide the recommendation for the personalized treatment plan.

20. The method of claim 1, further comprising:
determining a presence or absence of each mutation of a specific set of mutations based on the somatic mutations; and
determining a genotype-directed therapy for the subject based on the presences or absences.

21. The method of claim 1, further comprising evaluating the somatic mutations association with specific inherited conditions.

22. The method of claim 1, further comprising:
determining a ctDNA level based on the somatic mutations; and
evaluating a response to a treatment based on the ctDNA level or a remission status of a disease associated with the patient.

23. A system comprising:
a high-throughput sequencing system;
one or more processors; and
one or more computer-readable media storing instructions which, when executed by the one or more processors, cause the system to perform operations comprising:
performing whole genome sequencing using the high-throughput sequencing system on nucleic acids from a sample obtained from a subject to obtain raw data, wherein the raw data is stored in a specific format in a storage medium associated with the high-throughput sequencing system or in a local storage device communicatively coupled to the high-throughput sequencing system;
transmitting the raw data from the high-throughput sequencing system to a computing system, wherein the computing system comprises at least one processor and a memory, wherein the computing system is either a local server connected to the high-throughput sequencing system or a cloud-based server accessible via a network;
processing the raw data by the computing system to generate candidate variants and a set of feature values for each candidate variant of the candidate variants, wherein the processing comprises:
obtaining sequence reads from the raw data;
filtering the sequence reads based on a predetermined protocol to generate filtered sequence reads;
determining candidate variants based on the filtered sequence reads; and
determining the set of feature values for each candidate variant of the candidate variants based on the filtered sequence reads and the raw data, wherein the feature values are stored in a specific data structure;
processing the feature values by the computing system using a trained machine learning model to identify somatic mutations from the candidate variants, wherein the processing comprises:
loading the trained machine learning model into the memory of the computing system,
allocating memory and computational resources to execute the trained machine learning model, wherein the memory allocation comprises dynamic memory management for model execution, and wherein the computational resource allocation comprises assigning processing tasks to specialized accelerators of the computing system,
inputting the feature values into the trained machine learning model, and
generating, by the trained machine learning model, output data comprising the somatic mutations and associated metadata;
transmitting results based on the somatic mutations from the computing system to an end device, wherein the end device is communicatively connected to the computing system via the network; and
displaying a report on the end device based on the results.

24. A non-transitory computer-readable medium storing instructions which, when executed by one or more processors, cause the one or more processors to perform operations comprising:
performing whole genome sequencing using a high-throughput sequencing system on nucleic acids from a sample obtained from a subject to generate raw data, wherein the raw data is stored in a specific format in a storage medium associated with the high-throughput sequencing system or in a local storage device communicatively coupled to the high-throughput sequencing system;
transmitting the raw data from the high-throughput sequencing system to a computing system, wherein the computing system comprises at least one processor and a memory, wherein the computing system is either a local server connected to the high-throughput sequencing system or a cloud-based server accessible via a network;
processing the raw data by the computing system to generate candidate variants and a set of feature values for each candidate variant of the candidate variants, wherein the processing comprises:
obtaining sequence reads from the raw data;
filtering the sequence reads based on a predetermined protocol to generate filtered sequence reads;
determining the candidate variants based on the filtered sequence reads; and
determining the set of feature values for each candidate variant of the candidate variants based on the filtered sequence reads and the raw data, wherein the feature values are stored in a specific data structure;
processing the feature values by the computing system using a trained machine learning model to identify somatic mutations from the candidate variants, wherein the processing comprises:
loading the trained machine learning model into the memory of the computing system,
allocating memory and computational resources to execute the trained machine learning model, wherein the memory allocation comprises dynamic memory management for model execution, and wherein the computational resource allocation comprises assigning processing tasks to specialized accelerators of the computing system,
inputting the feature values into the trained machine learning model, and
generating, by the trained machine learning model, output data comprising the somatic mutations and associated metadata;
transmitting results based on the somatic mutations from the computing system to an end device, wherein the end device is communicatively connected to the computing system via the network; and
displaying a report on the end device based on the results.
